SEQUENCE LISTING (56 sequences)

SEQ ID NO: 1 - protein, 17 aa, Homo Sapiens
ALYYDLISSPDIHGTYK

SEQ ID NO: 2 - protein, 17 aa, Homo Sapiens
ALYYDLLSSPDLHGTYK

SEQ ID NO: 3 - protein, 11 aa, Homo Sapiens
CGALQGAVGNK

SEQ ID NO: 4 - protein, 11 aa, Homo Sapiens
DLLASVTAPQK

SEQ ID NO: 5 - protein, 11 aa, Homo Sapiens
DQQLGAGADER

SEQ ID NO: 6 - protein, 12 aa, Homo Sapiens
DTDTGALLFIGK

SEQ ID NO: 7 - protein, 12 aa, Homo Sapiens
DTDTGALLFLGK

SEQ ID NO: 8 - protein, 15 aa, Homo Sapiens
EIPDEISILLLGVAH

SEQ ID NO: 9 - protein, 11 aa, Homo Sapiens
ELLDTVTAPQK

SEQ ID NO: 10 - protein, 9 aa, Homo Sapiens
ELLDTVTAR

SEQ ID NO: 11 - protein, 17 aa, Homo Sapiens
ELPDELSLLLLGVAHFK

SEQ ID NO: 12 - protein, 8 aa, Homo Sapiens
FKGQWVTK

SEQ ID NO: 13 - protein, 7 aa, Homo Sapiens
GPLWPLR

SEQ ID NO: 14 - protein, 6 aa, Homo Sapiens
GQWVTK

SEQ ID NO: 15 - protein, 7 aa, Homo Sapiens
ITGKPIK

SEQ ID NO: 16 - protein, 6 aa, Homo Sapiens
IVFEKK

SEQ ID NO: 17 - protein, 13 aa, Homo Sapiens
KTSLEDFYLDEER

SEQ ID NO: 18 - protein, 14 aa, Homo Sapiens
LAAAVSNFGYDLYR

SEQ ID NO: 19 - protein, 15 aa, Homo Sapiens
LDLQEINNWVQAQMK

SEQ ID NO: 20 - protein, 15 aa, Homo Sapiens
LDLQELNNWVQAQMK

SEQ ID NO: 21 - protein, 7 aa, Homo Sapiens
LLTGNSR

SEQ ID NO: 22 - protein, 6 aa, Homo Sapiens
LPWPPR

SEQ ID NO: 23 - protein, 9 aa, Homo Sapiens
LQLCGTSGK

SEQ ID NO: 24 - protein, 12 aa, Homo Sapiens
LQSLFDSPDFSK

SEQ ID NO: 25 - protein, 9 aa, Homo Sapiens
LSYEGEVTK

SEQ ID NO: 26 - protein, 18 aa, Homo Sapiens
LTFPLDYHLNQPFLFVLR

SEQ ID NO: 27 - protein, 7 aa, Homo Sapiens
LTGKPLK

SEQ ID NO: 28 - protein, 7 aa, Homo Sapiens
LTQVEHR

SEQ ID NO: 29 - protein, 6 aa, Homo Sapiens
SLQEMK

SEQ ID NO: 30 - protein, 9 aa, Homo Sapiens
SSFVAPLEK

SEQ ID NO: 31 - protein, 30 aa, Homo Sapiens
SSTSPTTNVLLSPLSVATALSALSLGAEQR

SEQ ID NO: 32 - protein, 6 aa, Homo Sapiens
SVQELK

SEQ ID NO: 33 - protein, 7 aa, Homo Sapiens
SYGTRPR

SEQ ID NO: 34 - protein, 14 aa, Homo Sapiens
SYGTRPRVLTGNPR

SEQ ID NO: 35 - protein, 7 aa, Homo Sapiens
TESIIHR

SEQ ID NO: 36 - protein, 7 aa, Homo Sapiens
TESLLHR

SEQ ID NO: 37 - protein, 12 aa, Homo Sapiens
TSLEDFYLDEER

SEQ ID NO: 38 - protein, 10 aa, Homo Sapiens
TVQAVLTVPK

SEQ ID NO: 39 - protein, 12 aa, Homo Sapiens
VHLCESLNSNPR

SEQ ID NO: 40 - protein, 7 aa, Homo Sapiens
VLTGNPR

SEQ ID NO: 41 - protein, 8 aa, Homo Sapiens
VPMMSDPK

SEQ ID NO: 42 - protein, 12 aa, Homo Sapiens
VPMMSDPQAVLR

SEQ ID NO: 43 - protein, 22 aa, Homo Sapiens
VTQNLTLIEESLTSEFIHDIDR

SEQ ID NO: 44 - protein, 10 aa, Homo Sapiens
YGLDSDLSCK

SEQ ID NO: 45 - protein, 29 aa, Homo Sapiens
YGLDSDLSCKIAQLPLTGSMSIIFFLPLK

SEQ ID NO: 46 - protein, 418 aa, Homo Sapiens [sequence omitted]

SEQ ID NO: 47 - protein, 399 aa, Homo Sapiens
  DOMAIN (59)..(102): Active site of protein isoform -3 (PIF-3)
QNPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 48 - protein, 7 aa, Homo Sapiens
NLKSASR

SEQ ID NO: 49 - protein, 7 aa, Homo Sapiens
ILDPRGP

SEQ ID NO: 50 - protein, 8 aa, Homo Sapiens
QNPASPPE

SEQ ID NO: 51 - protein, 10 aa, Homo Sapiens
IVFEKKLRIK

SEQ ID NO: 52 - protein, 116 aa, Homo Sapiens
QNPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTAPQKN

SEQ ID NO: 53 - protein, 132 aa, Homo Sapiens
QNPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTAPQKNLKSASRIVFEKKLRIK

SEQ ID NO: 54 - protein, 268 aa, Homo Sapiens
KSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 55 - protein, 284 aa, Homo Sapiens
NLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 56 - protein, 30 aa, Homo Sapiens
SSMSPTTNVLLSPLSVATALSALSLGAEQR

(19) United States
(12) Patent Application Publication
(10) Pub. No.: US 2010/0223678 A1
(43) Pub. Date: Sep. 2, 2010

(54) protein isoforms of the pif-family and uses thereof

(76) Inventor: Christian Rohlff, Oxfordshire (GB)

Correspondence Address: KLAUBER & JACKSON, 411 HACKENSACK AVENUE, HACKENSACK, NJ, 07601, US

(21) Appl. No.: 11/989,828

(22) PCT Filed: Aug. 4, 2006
(86) PCT No.: PCT/GB2006/050232

§ 371 Date: Apr. 15, 2010

Related U.S. Application Data
(60) Provisional application No. 60/722,087, filed on Sep. 30, 2005.

(30) Foreign Application Priority Data
Aug. 4, 2005 (GB) 0516058.5

(51) Int. Cl. A61K 49/00; G01N 33/567; C07K 14/00; C07K 16/18; A61K 39/395; A61K 31/7088; G01N 33/53; C12Q 1/68; G01N 33/50; A61P 25/28

(52) U.S. Cl. 800/3; 435/7.21; 530/350; 530/387.1; 530/388.1; 424/130.1; 514/44 R; 514/44 A; 435/7.2; 435/6; 424/9.2; 435/7.1

(57) ABSTRACT

A method for screening for or diagnosis or prognosis of a neurological disorder associated with de-regulated glutamate signalling in a subject, for determining the stage or severity of such a neurological disorder in a subject, for identifying a subject at risk of developing such a neurological disorder, or for monitoring the effect of therapy administered to a subject having such a neurological disorder, said method comprising:
(a) analyzing a test sample of body fluid or tissue from the subject said sample comprising at least one Protein Isoform selected from the following Protein Isoform Families: PIF-1, PIF-2, and PIF-3 in a detectable amount; and
(b) comparing the abundance of said Protein Isoform(s) in the test sample or the abundance of said Protein Isoform(s) relative to another Protein Isoform with the abundance or relative abundance of said Protein Isoform(s) in a test sample from one or more persons free from neurological disorder, or with a previously determined reference range for that Protein Isoform in subjects free from neurological disorder, wherein a diagnosis of or a positive result in screening for or a prognosis of a more advanced condition of said neurological disorder is indicated by (i) decreased abundance or relative abundance of PIF-1 and/or (ii) increased abundance or relative abundance of PIF-2 and/or (iii) decreased abundance or relative abundance of PIF-3.

### 1. INTRODUCTION

The present invention relates to the identification of new Protein Isoforms that are associated with neurological disorders, in particular Alzheimer's disease, vascular dementia, schizophrenia, and depression, and their onset and development, and to their use for e.g., clinical screening, diagnosis, treatment, as well as for drug screening and drug development.

### 2. BACKGROUND OF THE INVENTION

Neurological disorders, such as Alzheimer's disease, vascular dementia, schizophrenia, and depression, are often difficult to diagnose as the presentation of the disease differs highly from individual to individual. It would be highly desirable to measure a substance or substances in body samples, such as samples of brain tissue, cerebrospinal fluid (CSF), blood or urine, that would lead to a positive diagnosis of a condition or that would help to exclude a particular disease from the differential diagnosis.

### Alzheimer's Disease

Alzheimer's disease (AD) is an increasingly prevalent form of neurodegeneration that accounts for approximately 50-60% of the overall cases of dementia among people over 65 years of age. It currently affects an estimated 15 million people worldwide and owing to the relative increase of elderly people in the population its prevalence is likely to increase over the next 2 to 3 decades. Alzheimer's disease is a progressive disorder with a mean duration of around 8.5 years between onset of clinical symptoms and death. Death of pyramidal neurons and loss of neuronal synapses in brains regions associated with higher mental functions result in the typical symptomology, characterized by gross and progressive impairment of cognitive function (Francis et al., 1999, J. Neurol. Neurosurg. Psychiatry 66:137-47).

Currently, a diagnosis of Alzheimer's disease requires a careful medical history and physical examination; a detailed neurological and psychiatric examination; laboratory blood studies to exclude underlying metabolic and medical illnesses that masquerade as AD; a mental status assessment and formal cognitive tests; and a computed tomographic scan or magnetic resonance image of the brain (Growdon, J H., 1995, Advances in the diagnosis of Alzheimer's disease. In: Iqbal, K., Mortimer, J A., Winblad, B., Wisniewski, H M eds Research Advances in Alzheimer's Disease and Related Disorders. New York, N.Y.: John Wiley & Sons Inc. 1995:139-153).

Current candidate biomarkers for Alzheimer's disease include: (1) mutations in presenilin 1 (PS1), presenilin 2 (PS2) and amyloid precursor protein (APP) genes; (2) the detection of alleles of apolipoprotein E (ApoE); and (3) altered concentrations of amyloid B-peptides (AB), tau protein, and neuronal thread protein (NTP) in the CSF. See, e.g., Neurobiology of Aging 19:109-116 (1998) for a review. Mutations in PS1, PS2 and APP genes are indicative of early-onset familial Alzheimer's disease. However, early-onset familial Alzheimer's disease is relatively rare; only 120 families worldwide are currently known to carry deterministic mutations (Neurobiology of Aging 19:109-116 (1998)). The detection of the ε4 allele of ApoE has been shown to correlate with late-onset and sporadic forms of Alzheimer's disease. However, ε4 alone cannot be used as a biomarker for Alzheimer's disease since ε4 has been detected in many individuals not suffering from Alzheimer's disease and the absence of ε4 does not exclude Alzheimer's disease (Neurobiology of Aging 19:109-116 (1998)).

A decrease in the AB peptide AB42 and an increase in tau protein in the CSF of Alzheimer's disease have been shown to correlate with the presence of Alzheimer's disease (Neurobiology of Aging 19:109-116 (1998)). However, the specificity and sensitivity of Aβ42 and tau protein as biomarkers of Alzheimer's disease are modest. For example, it has been difficult to determine a cut-off level of CSF tau protein that is diagnostically informative. Also, elevated levels of NTP in the CSF of postmortem subjects have been shown to correlate with the presence of Alzheimer's disease (Neurobiology of Aging 19:109-116 (1998)).

Acetylcholinesterase inhibitors, such as tacrine, donepezil, rivastigmine, and galantamine are currently the main treatment available for AD; however, there is a large variation in the response of patients to therapy.

Memantine belongs to a new class of drugs that blocks excessive glutamate receptor activity with out disrupting normal activity. Glutamate is an excitatory amino acid (EAA) neurotransmitter (release of glutamate by one neuron stimulates activity in its neighbours) involved in the neurotoxic events leading to cell death after CNS trauma and ischemia and in some neurodegenerative disorders. Too much glutamate is extremely toxic. It is thought that much of the brain damage that occurs following stroke or in dementing illnesses, like Huntington's disease, is the result of excessive glutamate activity in the brain. During pathological activation such as that occurring in Alzheimer's disease may lead to progressive deficits in cognitive functions. The over activation of glutamate receptors may lead to damage of neurons and be responsible for both cognitive deficits and neuronal loss in neurodegenerative dementias (Lipton S A Curr Alzheimer Res. (2005)2:155-65). Memantine has been approved for use in Europe and is under review by the U.S. FDA. Beside Alzheimer's disease, Memantine is currently in trials for dementia and depression. A series of second generation Memantine derivatives are currently in development.

### Schizophrenia

Schizophrenia is characterized by episodes of positive symptoms such as delusions, hallucinations, paranoia and psychosis and/or negative symptoms such as flattened affect, impaired attention, social withdrawal, and cognitive impairments (Ban et al, Psychiatr. Dev. (1984) 2:179-199). It afflicts 1.1% of the U.S. population and imposes a disproportionately large economic burden due to long-term expenditures for hospitalisation, treatment and rehabilitation, and lost productivity. Effective treatments used early in the course of schizophrenia can help reduce the costs associated with this illness.

The relative contribution of genetic and environmental factors to the disease etiology remain uncertain, although an increased prevalence of schizophrenia has been demonstrated in family and twin studies (Kendler Am. J. Psychiatry (1983) 140:1413-1425) and resulted in the identification of candidate chromosomes including chromosome 6 and 22 and several candidate genes, such as the dopamine D3 receptor gene (Murphy et al, J Mol Neurosci (1996) 7:147-57). However, volumetric losses in the cerebral hemisphere, as well as changes in physiologic and neuropsychological performance deficits, such as a decreased prefrontal regional cerebral blood flow in the same twin studies, suggest a significant contribution of nonheritable factors to the pathogenesis of schizophrenia (Goldberg Psychiatry Res. (1994) 55:51-61).

Currently diagnosis of schizophrenia remains clinically based on the presence of certain types of hallucinations, delusions and thought disorders (Andreasen Lancet (1995) 346:477-481). It is made on the basis of a careful clinical interview and mental status examination according to international established manuals, in particular the DSM-IV or ICD 10. The core clinical symptoms comprise formal thought disorders, delusions, hallucinations (also summarized as positive symptoms), and negative symptoms such as lack of drive and affect flattening. Neuroimaging techniques such as magnetic resonance imaging or positron emission tomography show subtle changes of the frontal and temporal lobes and the basal ganglia (Buchsbaum, Schiz. Bull. (1990) 16:379-389) in the majority of patients. Since these alterations are of little value for the diagnosis, treatment, or prognosis of the disorder in individual patients the role of the neuroimaging techniques mentioned above is by and large restricted to the exclusion of other conditions which may be accompanied by schizophrenic symptoms such as brain tumors, hemorrhages, or—in combination with chemical parameters obtained in CSF-samples—infections of the central nervous system.

Neuroleptic agents are essential for the treatment of schizophrenia. While typical neuroleptics affect primarily the dopaminergic system, newer atypical neuroleptics also afflict serotonergic synapses. In general, the latter have greater effects on negative symptoms and cause less extrapyramidal side effects than typical neuroleptic compounds. It is generally accepted that early and continuous neuroleptic treatment may improve the outcome of the disorder. Nevertheless, regardless of the particular drug used, neuroleptic treatment is still considered to be solely symptomatic and does not inhibit the causes of the disorder.

Currently schizophrenia has no objective biochemical markers useful for diagnosis and prognosis in living patients. Many CNS pathologies involve increased neuronal loss and such neuronal loss or impaired synaptogenesis may result in disease associated alterations of neuronal and CSF proteins. Synaptic pathologies have been implicated in schizophrenia (Heinonen et al, Neuroscience (1995) 64:375-384; Benes Schiz. Bull. (1993) 19:537-549). Consequently, it is not surprising that changes in synaptic proteins such as SNAP 25 (Thompson et al, Neuropsychopharmacology (1999) 21:717-22), neurotensin (Sharma et al, Am J Psychiatry (1997) 154:1019-21) and N-CAM (Vawter et al, Schizophr Res (1998) 34:123-31) have been detected in CSF of Schizophrenia patients. N-CAM levels are altered in affected twins and not in healthy siblings (Poltorak et al, Brain Res (1997) 751:152-4) suggesting they may be directly linked to the pathogenesis of Schizophrenia. Such DAPs may provide important insights into disease pathology and opportunities for better diagnosis and treatment strategies. However, these changes may also occur in other diseases, such as the elevation of α-2 haptoglobin in schizophrenia and Alzheimer's disease (Johnson et al, Applied and Theoretical Electrophoresis (1992) 3:47-53) and elevated SNAP-25 levels in Schizophrenia and bipolar patients (Thompson op. cit). Therefore, the specificity and the sensitivity of distinguishing individual neurological disorders as well as acute and chronic CNS disease may require the selection of a repertoire of disease associated proteins rather than an individual protein.

The glutamate antagonist lamotrigine shows benefits in patients with treatment-resistant schizophrenia. The anticonvulsant controlled both positive and general psychopathological symptoms in schizophrenia patients. It has been shown previously that ketamine-induced positive schizophreniform symptoms in healthy subjects can also be controlled by lamotrigine (Tiihonen J et al. Biol Psychiatry (2003) 54:1241-1248).

### Depression

Depression is one of the most common, severe and often life threatening neuropsychiatric disorders, thought to affect 9.5% of the population in the US within a given 1-year period. It can be subdivided into major or unipolar (UP) depression and bipolar (BP) depression. Suicide is the cause of death in 10% to 20% of individuals with either bipolar or recurrent disorders, and the risks of suicide in bipolar disorder may be higher than those in unipolar depression (reviewed by Simpson and Jamison, J Clin Psychiatry 1999, 60, 53-56). BP is characterized by episodes of elevated mood (mania) and depression (Goodwin et al. 1990, Manic Depressive Illness, Oxford University Press, New York).

BP depression can be further subdivided into BP I, when the patients has experienced one or more episodes of mania, or BP II, when a patient has experienced a hypomanic episode but has not met the criteria for a full manic episode. BP often also co-segregates in families with unipolar major depressive disorder (MDD), which has a broadly defined phenotype (Freimer and Reus, 1992, in The Molecular and Genetic Basis of Neurological Disease, Rosenberg et al. Eds., Butterworths, New York, pp. 951-965; McInnes and Freimer, 1995, Curr. Opin. Genet. Develop., 5, 376-381). The identification of proteins and Protein Isoforms that are associated with the onset and progression of various forms of depression would be desirable for the effective diagnosis, prognosis and treatment of afflicted individuals.

Major mood disorders are also associated with many other deleterious health-related effects and the costs with disability and premature death represent an economic burden of $43 billion annually in the United States alone. Despite the devastating impact of these disorders on the lives of millions, there is still uncertainty about the differential diagnosis of depression in the presence of these disorders (Goldman et al. 1999, J Gen Med 14, 569-80; Schatzberg 1998, J ClinPsychiatry, 59, suppl 6:5-12; Goodwin and Jamison, 1990 Manic-depressive illness, New York, Oxford University Press).

Major depression is a syndromal diagnosis: on the basis of the patient's medical history and physical examination, it may be appropriate to consider other psychiatric disorders and general medical conditions (Goldman et al. J Gen Intern Med 1999, 14, 569-580) but very limited knowledge exists concerning their etiology and pathophysiology (Ikonomov et al. 1999, Am J Psychiatry, 156, 1506-1514). Genetic segregation analyses and twin studies suggest genetic element for BAD (Bertelson et al. 1977, Br. J. Psychiat. 130, 330-351; Freimer and Reus, 1992, in The Molecular and Genetic Basis of Neurological Disease, Rosenberg et al. Eds., Butterworths, New York, pp. 951-965; Pauls et al. 1992, Arch. Gen. Psychiat. 49, 703-708). Although several localizations for genes involved in BP have been proposed on chromosome 18p and 21q and candidate regions for possible gene locations are now well defined, no genes associated with the disease have been identified yet (Berrettini et al. 1994, Proc. Natl. Acad. Sci., USA 91, 5918-5921; Murray et al. 1994, Science265, 2049-2054; Pauls et al. 1995, Am. J. Hum. Genet. 57, 636-643; Maier et al. 1995, Psych. Res. 59, 7-15).

Major depression is a frequent diagnosis in patients evaluated for both cognitive and affective disorders and many depressed patients, in fact, are clinically characterized by cognitive impairments (Emery and Oxman, 1992, Am J Psychiatry, 149, 305-317).

Current therapeutics can be categorized into the following major classes of agents: mood stabilizers: lithium, divalproex, carbamazepine, lamotrigine; antidepressants: tricyclic antidepressants (eg. Desipramine, chlorimipramine, nortriptyline), selective serotonin re uptake inhibitors (SSRIs including fluoxetine (Prozac), sertraline (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: clozapine, olanzapine, risperidone. However, the cellular and molecular basis for the efficacy of currently used mood-stabilizing and mortality-lowering agents remains to be fully elucidated (Manji et al. 1999, J Clin Psychiatry, 60, 27-39). A significant number of patients respond poorly to existing therapies such as lithium, while many others are helped but continue to suffer significant morbidity (Chou 1991, J Clin Psychopharmacol 11, 3-21). The recognition of the significant morbidity and mortality of the severe mood disorders, as well as the growing appreciation that a significant percentage of patients respond poorly to existing treatments, has made the task of developing new therapeutic agents that work quickly, potently, specifically, and with fewer side effects one of major public health importance (Bebchuk et al. Arch Gen Psychiatry 2000 57,95-7).

The glutamate antagonist lamotrigine has been approved by the FDA for the for the maintenance treatment of adults with Bipolar I Disorder to delay the time to occurrence of mood episodes (depression, mania, hypomania, mixed episodes) in patients treated for acute mood episodes with standard therapy. Bipolar disorder, a serious, chronic illness marked by disabling mood swings from high (manic) to low (depressed) states, (1) is one of the most common mental illnesses in the United States (Bhagwagar Z et al., Expert Opin Pharmacother. (2005) 8:1401-8).

### Vascular Dementia

Vascular dementia is the second most common cause of dementia in the US and Europe and is a very heterogeneous disease with many factors contributing to the overall pathogenesis. Eight major types of vascular dementia have been identified 1)-Multi-infarct dementia secondary to large cerebral emboli, 2)-Strategically placed infarctions causing dementia, 3)-Multiple subcortical lacunar lesions secondary to atherosclerosis or degenerative arteriolar changes, 4)-Binswanger's disease (arteriosclerotic subcortical leukoencephalopathy), 5)-Mixtures of types 1, 2 and 3, 6)-Haemorrhagic lesions causing dementia, 7)-Subcortical dementia secondary to hereditary factors, and 8)-Mixtures of dementia of the Alzheimer's type and vascular dementia (Konno et al. Drugs Aging (1997) 11:361-73). A great need exists for an improved diagnosis of vascular dementia. Today, the clinician depends on clinical examinations, the patient's history, and, possibly, brain imaging to recognize signs of vascular dementia such as cerebrovascular damage. Currently diagnosis combines several methods including brain imaging of the injured site via computed tomography (CT) or magnetic resonance imaging (MRI) (Kistler et al. Stroke (1984) 15:417-26), duplex and transcranial Doppler methods (Comerota et al. Surgery (1981) 6:718-29), and positron emission tomography (Frackowiak and Kjaellman Neurol Clin North Am (1983) 1:183-200).

Numerous clinical criteria are used in the diagnosis of vascular dementia VD, resulting in variations the frequency of VD diagnosis depending on the applied criteria (4 clinical definitions of VD are currently used: the Hachinski Ischemic Score (HIS), the Alzheimer Disease Diagnostic and Treatment Centers (ADDTC), National Institute of Neurological Disorders and Stroke-Association Internationale pour la Recherche et l'Enseignement en Neurosciences (NINDS-AIREN), and Diagnostic and Statistical Manual of Mental Disorders, Fourth Edition (DSM-IV)) (Chui et al. Arch Neurol (2000) 57:191-6). Even though standardized neuropsychological assessments facilitate the differential diagnosis of vascular dementia from Lewy Body dementia and Alzheimer's disease (Ballard et al. Dement Geriatr Cogn Disord (1999) 10:104-8), diagnostic criteria for vascular dementia still require validation (Nyenhuis and Gorelick J Am Geriatr Soc (1998) 46:1437-48).

In the majority of neurological disorders like vascular dementias, little is known about a link between changes at a cellular and/or molecular level and nervous system structure and function. In an acute stroke a quick assessment of the cause, severity and chance of progression or recurrence is necessary to ensure an optimal treatment to stabilize or reverse the effects. Few biochemical changes have been identified in vascular dementia. Consequently the identification and characterization of cellular and/or molecular causative defects and neuropathologies are necessary for improved treatment of vascular dementia associated disorders. Due to the possibility of worsening or recurrence, speedy diagnosis would be of great benefit, in particular to categorize the patient as follows:

1. Stroke versus non-stroke such as cerebral tumors and subdural hematoma
2. Hemorrhage versus infraction
3. Specific pathophysiological subtypes of cerebral infarction (Donnan Lancet (1992)339:473).

In particular cerebrovascular dementia often coexists with other causes of dementia (Erkinjuntti Int Psychogeriatr (1997) 9 Suppl 1:51-8; discussion 77-83) complicating a proper diagnosis and effective treatment strategies. The majority of vascular dementias are caused by both genetic and environmental factors (Plassman and Breitner J Am Geriatr Soc (1996) 44:1242-50), although an increased prevalence of vascular dementia has been demonstrated in the cerebral arteriopathy syndrome, a genetic form of vascular dementia (Salloway and Hong J Geriatr Psychiatry Neurol (1998) 11:71-7), apolipoprotein E gene polymorphism in Binswanger's disease and vascular dementia (Higuchi et al. Clin Genet (1996) 50:459-61) and hereditary cystatin C amyloid angiopathy (HCCAA) in Icelandic patients and hereditary cerebral haemorrhage with amyloidosis, Dutch type (HCHWA-D) (Wang et al. APMIS (1997) 105:41-7).

Current treatments of vascular dementia include antithrombic therapies (Crowth and Ginsberg in Stroke, Pathophysiology, Diagnosis, and Management Eds. Barnett, Mohr et al. Year, Churchill Livingston, a division of Harcourt Brace & Company), thrombolytic and defibrinogenating agents (Brott and Hacke in Stroke, supra), antiplatelet agents (Weksler in Stroke, supra) and neuroprotective agents (Gluckmann and Gunn in Neuroprotection in CNS diseases, Eds. Baer and Beal Year, Marcel Dekker, Inc. New York).

Glutamate is the main excitatory neurotransmitter in the mammalian CNS, were fast postsynaptic depolarisation is induced by the activation of AMPA (α-amino-3-hydroxy-5-methyl-4-isoxazolepropionic acid) receptors. In addition to their important physiological role, excessive AMPA receptor stimulation is also a hallmark of excitotoxicity-related diseases, like ischaemic stroke. Non-competitive AMPA receptor antagonists like 2,3-benzodiazepines or the novel neuroprotectant BIM 561 have been proposed for the treatment of stroke (Weiser, T, Current Drug Targets—CNS & Neurological Disorders (2005) 4: 153-159).

### Multiple Sclerosis

Multiple sclerosis (MS) is an inflammatory demyelinating disorder with preservation of the axons and considered the most common cause of neurologic disability in young adults. Although the mean age at onset for MS is 30 years, there are two prevalent age groups. The majority of patients are between 21 and 25 years at onset and a smaller percentage are 41 to 45 years of age. In the western world, more than 80 per 100,000 population are affected (Kurtzke, J. F. (1980) Neurology (N.Y.), 7:261-279). Several twin studies in Canada and the UK revealed that monozygotic twins are concordant in the order of 30%, compared to 2% in dizygotic twins and siblings (Ebers, G. C. et al. (1986) New Engl J Med, 315:1638-42; Mumford, C. J. et al. The British Isles survey of multiple sclerosis in twins. (1994) Neurology, 1004:44, 11-15) and the current evidence suggests that multiple genes may interact to increase susceptibility to MS (Noseworthy (1999) Nature 399:suppl. A40-A47).

While genetics and genotyping may help to define the heritable risk for MS, the utility for diagnosis, prognosis and treatment of MS may be considerably less. It remains still unknown whether MS is a single disease and how it relates to the less common inflammatory-demyelinating CNS syndromes including neuromyelitis optica, transverse myelitis, Balo's concentric sclerosis, the Marburg variant of acute MS and acute disseminated encephalomyelitis (Noseworthy, Progress in determining the causes and treatment of multiple sclerosis, (1999) Nature 399:suppl. A40-A47).

Post-mortem examination of MS patients revealed the presence of multiple lesions (plaques) in the central nervous system characterized by demyelination, with relative preservation of axons, as well as gliosis and different degrees of inflammation. Although there are certain sites of predilection including the optic nerves, the spinal cord, and the periventricular regions, any part of the brain or cord can be affected (Lumsden, C. E. (1970) In Vinken P. J. Bruyn, G W, eds., Handbook of Clinical Neurology. Vol. 9. Amsterdam, Noth Holland, P.P. 217-309). In the majority of inflammatory neurological disorders like MS, little is known about a link between changes at a cellular and/or molecular level and nervous system structure and function.

The diagnosis remains a clinical one. Diagnosis requires the demonstration of lesions disseminated in time and space and the exclusion of other conditions that may produce the same clinical picture. Clinical classification of MS, known as the Poser criteria, includes abnormalities of evoked response and MRI, and immunologic abnormalities in the CSF (Poser, C. M. et al. (1983) Ann Neurol 13: 227-231). Symptoms of MS at presentation vary among studied populations but include sensory symptoms in 24% of patients, optic neuritis in of 31% patients, limb weakness in 17% of patients and brain stem and cerebellar symptoms 25% of patients (Thompson, A. J. et al. (1986) Q.J. Med. 225:69-80). Consequently MS has a wide range of clinical presentations and courses, and the clinical course of any given patient is unpredictable. In the majority of MS patients it begins with a relapsing and remitting course, where episodes of neurological dysfunction last several weeks. Over the course of disease remissions tend be less than complete and patients pass into a progressive phase (secondary progression). During this phase of the disease patients develop severe irreversible disabilities. About one-third of patients have benign MS, which does not develop secondary progression. Approximately 10% of patients develop progressive disability from onset without relapses and remissions (primary progressive MS). Few biochemical changes have been identified in MS. Consequently the identification and characterization of cellular and/or molecular causative defects and neuropathologies are necessary for improved treatment of neurological disorders. Due to the possibility of worsening or recurrence, speedy diagnosis would be of great benefit, in particular to categorise the patient as follows:

1. Benign versus progressive MS

2. Primary versus secondary progressive MS

3. Specific pathophysiological subtypes of primary and secondary progressive MS

Treatments strategies have three aims: 1, to modify the course of the disease, 2, to affect severity and duration of relapse and 3, symptomatic treatment and neurorehabilitation.

Currently MS has no objective biochemical markers useful for diagnosis and prognosis in living patients. The identification of disease specific proteins (DSPs) in the CSF of MS patients may provide important insights into disease pathology and opportunities for better diagnosis and treatment strategies. Isoelectric focusing of cerebrospinal fluid (CSF) from MS patients revealed the presence of oligoclonal bands in 95% of patients with MS (McLean et al. (1990) Brain, 113:1269-89). However, similar to MRI, this finding is not specific to MS patients and can also be detected in other neurological disorders including Guillain-Barre syndrome, sarcoidosis and chronic meningitis. Therefore, the specificity and the sensitivity of distinguishing individual neurological disorders as well as acute and chronic CNS disease may require the selection of a repertoire of disease-associated proteins rather than an individual protein.

Histopathological reports of multiple sclerosis and its animal models have shown evidence of a link between axonal injury in active lesions and impaired glutamate metabolism and glutamate levels are elevated in certain brain regions of patients with multiple sclerosis (Srinivasan R et al., Brain. (2005) 128:1016-25). Multiple sclerosis patients have toxic levels of glutamate in their spinal fluid and compounds that block glutamate, which were being tested as stroke treatments, also show efficacy in experimental models of multiple sclerosis (Smith T, Nature Med (2000) 6: 62; Pitt D et al. Nature Med (2000) 6: 67).

### Overview

Due to the time consuming nature of the existing, largely inadequate, tests for the above neurological and neuropsychiatric conditions, and their expense, it would be highly desirable to be able to measure a substance or substances in samples of body tissue or fluid such as brain tissue, CSF, blood (eg serum) or urine that would either:
indicate that a subset of neurological conditions is to be included or excluded from the diagnosis
lead to the positive diagnosis of one of these neurological conditions (or conversely allow it to be excluded from the list of potential diseases).

In addition, although genetics and genotyping may help to define the heritable risk for these conditions, their utility for diagnosis, prognosis and treatment of these conditions may be considerably less. In vivo post-translational modifications and processing of proteins are indeed highly implicated in such conditions and can be monitored only by techniques able to detect and quantify proteins directly.

### 3. SUMMARY OF THE INVENTION

The present invention provides methods and compositions for screening, diagnosis and treatment of neurological disorders including Alzheimer's disease, Schizophrenia, depression, multiple sclerosis, and vascular dementia, and for screening and development of drugs for treatment of the above conditions.

A first aspect of the invention provides methods for identification of neurological disorders that comprise analyzing a sample, for example of cerebrospinal fluid (CSF) or of brain tissue, for example by two-dimensional electrophoresis, to detect the presence or level of at least one Protein Isoform of the invention as disclosed herein, or any combination thereof. These methods are also suitable for clinical screening, prognosis, monitoring the results of therapy, for identifying patients most likely to respond to a particular therapeutic treatment, drug screening and development, and identification of new targets for drug treatment.

A second aspect of the invention provides antibodies, e.g., monoclonal and polyclonal and chimeric (bispecific) antibodies or other affinity reagents such as Affibodies capable of immunospecific binding to a Protein Isoform of the invention.

A third aspect of the invention provides kits that may be used in the above recited methods and that may comprise single or multiple preparations, or antibodies (or other affinity reagents eg Affibodies), together with other reagents, labels, substrates, if needed, and directions for use. The kits may be used for diagnosis of disease, or may be assays for the identification of new diagnostic and/or therapeutic agents.

A fourth aspect of the invention provides methods of treating neurological disorder, comprising administering to a subject a therapeutically effective amount of an agent that modulates (e.g., upregulates or downregulates) the expression or activity (e.g. binding activity), or both, of a Protein Isoform of the invention in subjects having neurological disorder.

A fifth aspect of the invention provides methods of screening for agents that modulate (e.g., upregulate or downregulate) a characteristic of, e.g., the expression or binding activity, of a Protein Isoform of the invention, an analog thereof, or a related polypeptide.

Other objects and advantages will become apparent from a review of the ensuing detailed description taken in conjunction with the following illustrative drawings.

### 4. BRIEF DESCRIPTION OF THE FIGURES AND SEQUENCE LISTINGS

FIG. 1 illustrates the amino acid sequence of the precursor protein from which the new Protein Isoforms of the invention are derived. The signal sequence is underlined.

FIG. 2 illustrates the PIF-1 Protein Isoform Family of the invention which corresponds to the mature form of the precursor protein from FIG. 1. The cleavage site identified by the present inventors is single-underlined and the active site within the PIF-3 Protein Isoform Family (N-ter to the cleavage site) is in bold and double-underlined.

FIG. 3 illustrates the relative abundancies of one representative from each of the 3 Protein Isoform Families of the invention in the CSF of normal controls and of Schizophrenia patients.

FIG. 4 illustrates the relative abundancies of PIF-1 (FIGS. 4a, 4b, 4c, 4d, 4e), PIF-2

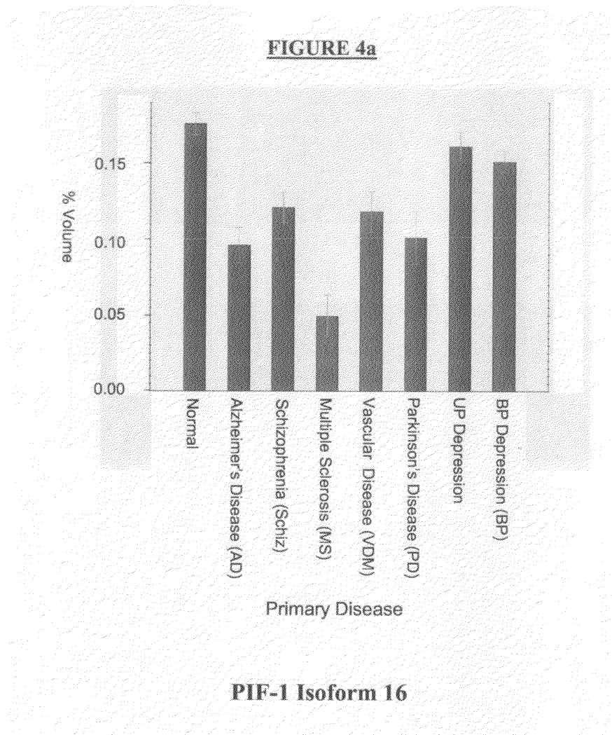

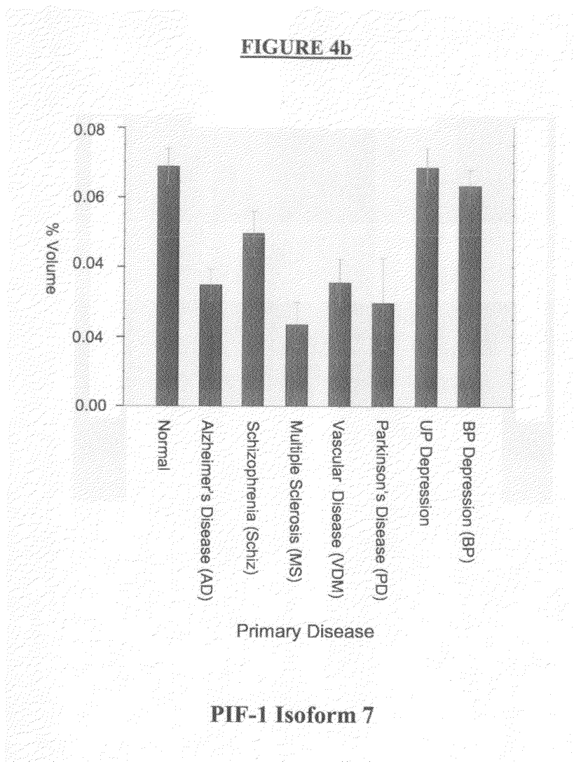

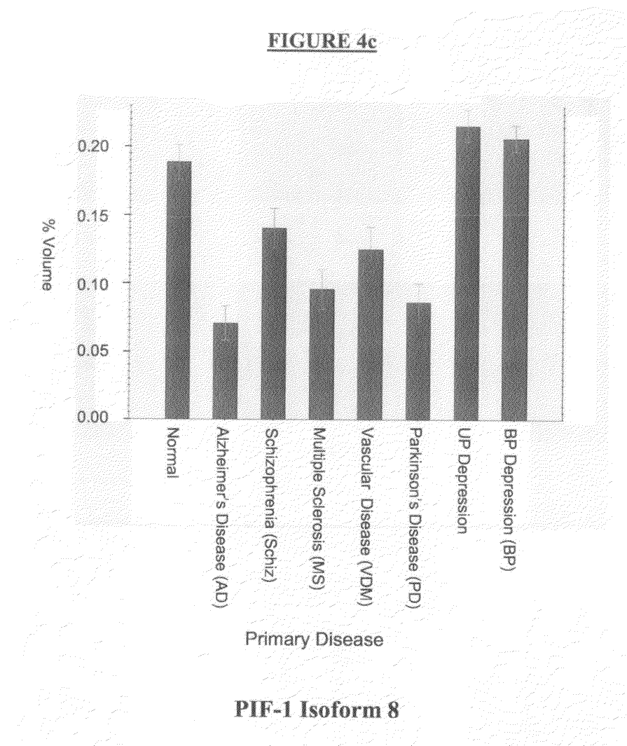

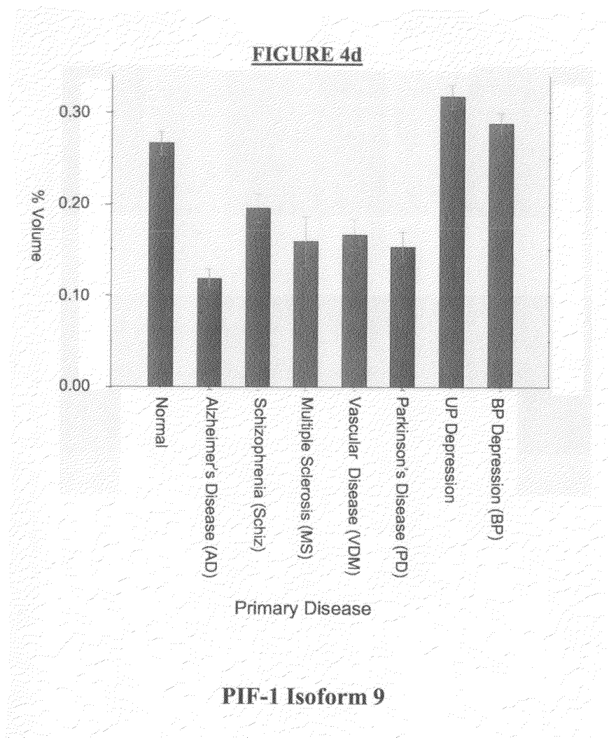

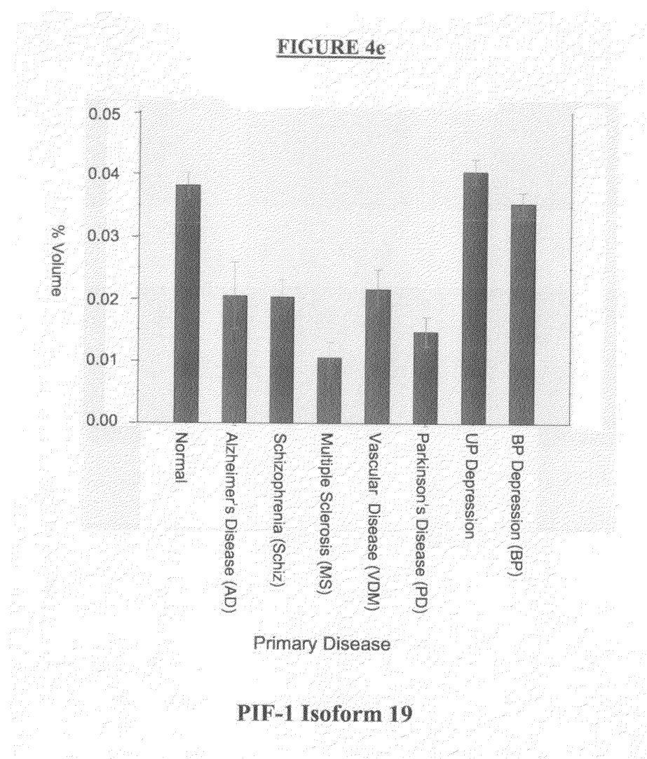

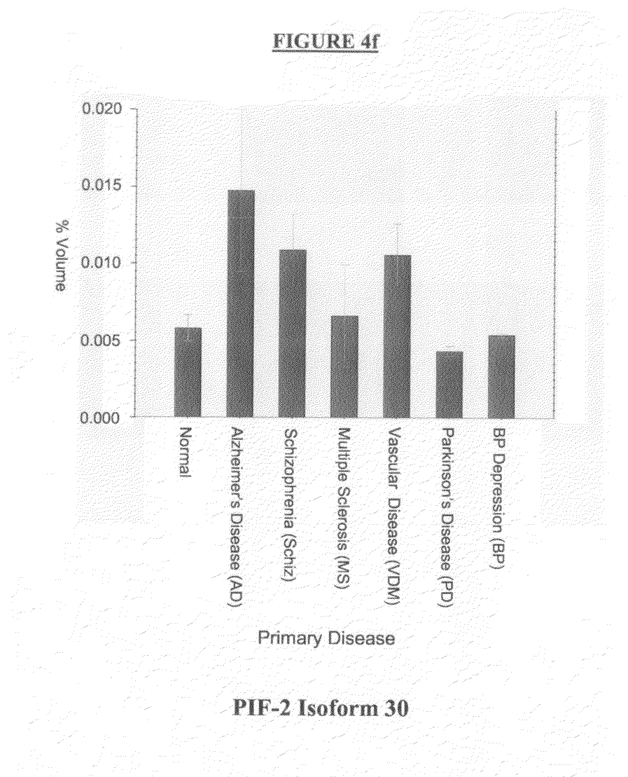

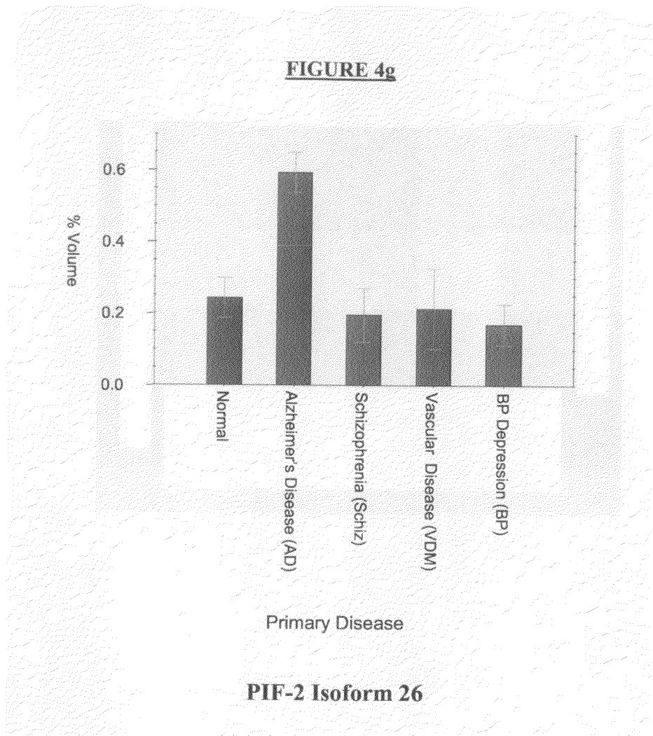

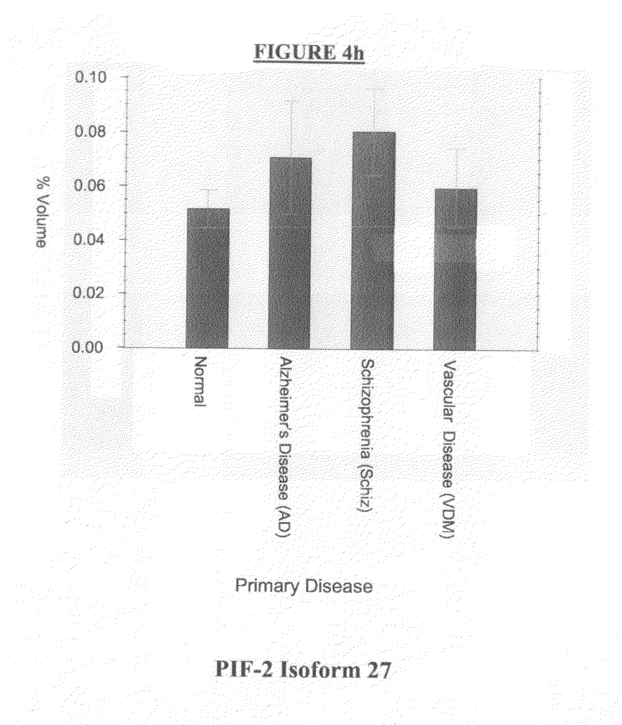

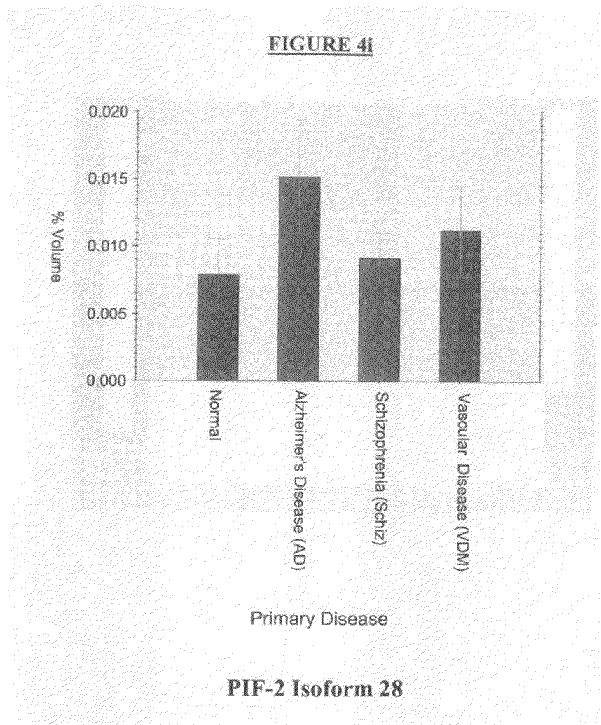

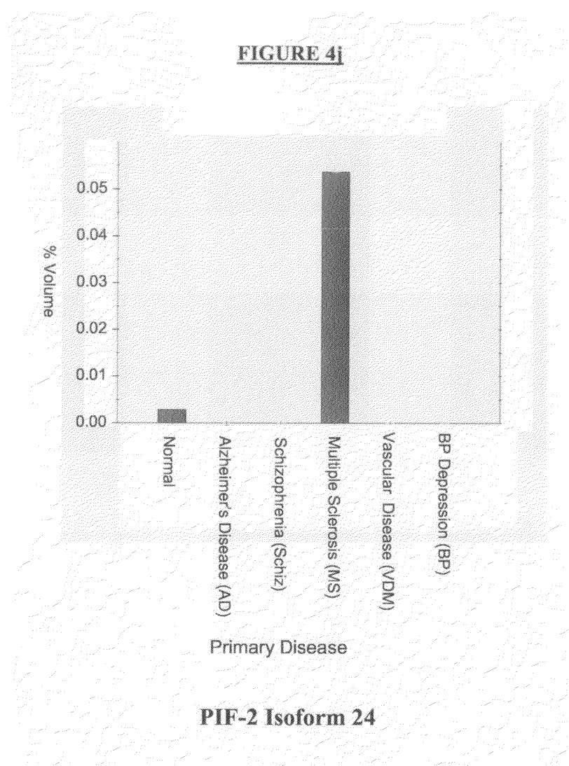

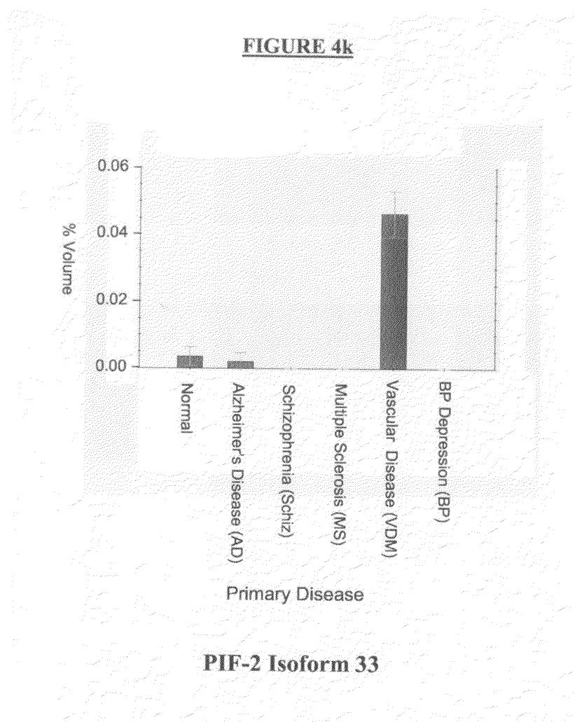

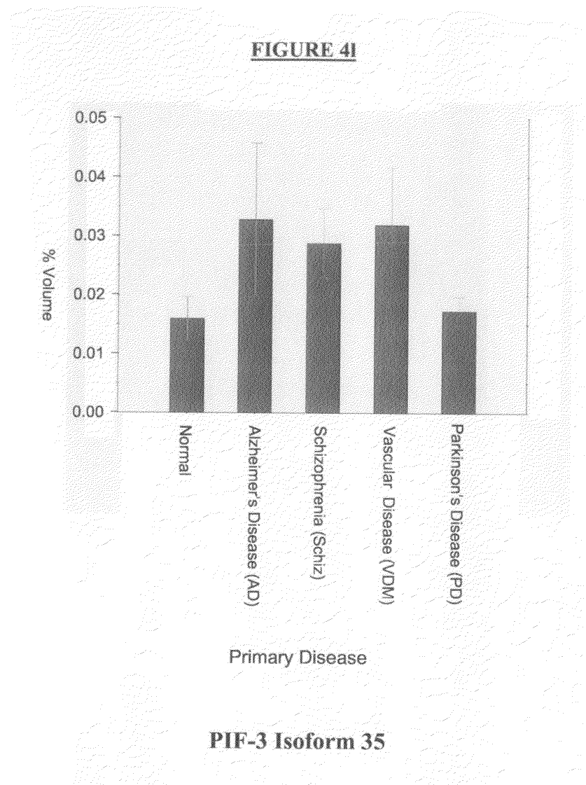

(FIGS. 4f, 4g, 4h, 4i, 4j, 4k) and PIF-3 (FIG. 41), each figure representing a different family member, in the CSF of normal controls and of patients having neurological disorders.

SEQ ID NOs: 1 to 45 and 56 correspond to tryptic digest sequences and other fragments described in Table 1 and elsewhere in the specification.

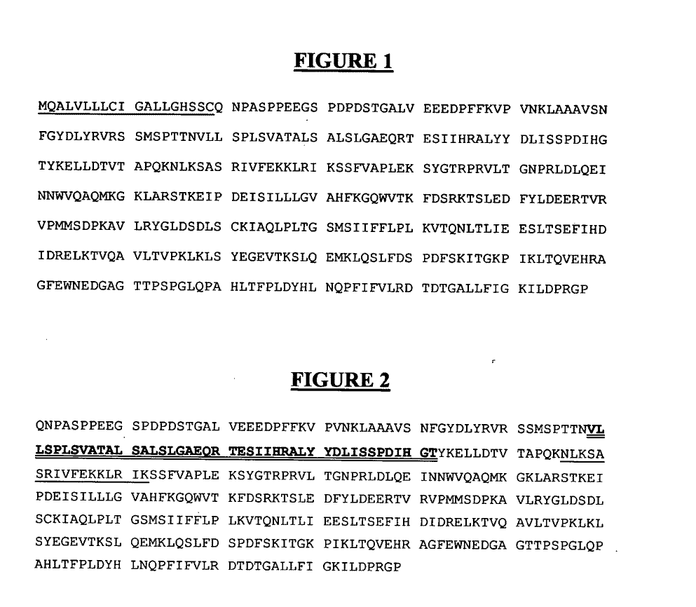

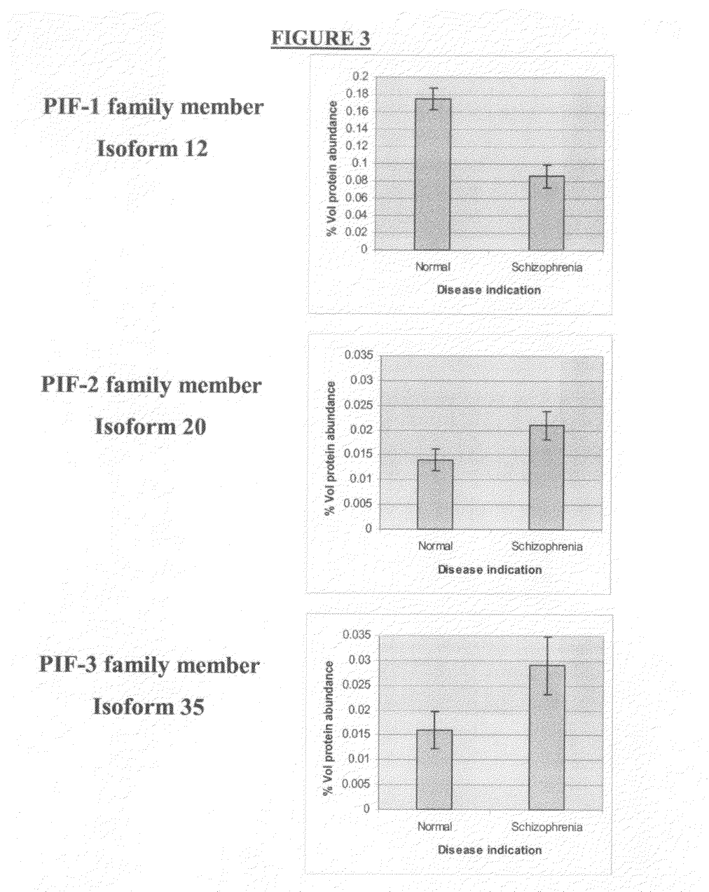

SEQ ID NO: 46 is the amino acid sequence of the precursor protein from which the new protein isoforms of the invention are derived (as provided in FIG. 1).

SEQ ID NO: 47 is the amino acid sequence of the mature form of the precursor protein from which the new protein isoforms of the invention are derived (as provided in FIG. 2).

SEQ ID NO: 48 is the N-terminus of an exemplary amino acid sequence for protein isoforms from the PIF-2 family.

SEQ ID NO: 49 is the C-terminus of an exemplary amino acid sequence for protein isoforms from the PIF-2 family.

SEQ ID NO: 50 is the N-terminus of an exemplary amino acid sequence for protein isoforms from the PIF-3 family.

SEQ ID NO: 51 is the C-terminus of an exemplary amino acid sequence for protein isoforms from the PIF-3 family.

SEQ ID NO: 52 is an exemplary sequence of a protein isoform from the PIF-3 family.

SEQ ID NO: 53 is an exemplary sequence of a protein isoform from the PIF-3 family.

SEQ ID NO: 54 is an exemplary sequence of a protein isoform from the PIF-2 family.

SEQ ID NO: 55 is an exemplary sequence of a protein isoform from the PIF-2 family.

### 5. DETAILED DESCRIPTION OF THE INVENTION

The present invention described in detail below provides Protein Isoforms and corresponding methods, compositions and kits useful, e.g., for screening, diagnosis and treatment of neurological disorder in a mammalian subject, and for drug screening and drug development. The invention also encompasses the administration of therapeutic compositions to a mammalian subject to treat or prevent a neurological disorder. The mammalian subject may be a non-human mammal, but is preferably human, more preferably a human adult, i.e. a human subject at least 21 (more particularly at least 35, at least 50, at least 60, at least 70, or at least 80) years old. The methods and compositions of the present invention are useful for screening, diagnosis and treatment of a living subject, but may also be used for postmortem diagnosis in a subject, for example, to identify family members of the subject who are at risk of developing the same disease.

The following definitions are provided to assist in the review of the instant disclosure.

### 5.1. Definitions

“Diagnosis” refers to diagnosis, prognosis, monitoring, selecting participants in clinical trials, and identifying patients most likely to respond to a particular therapeutic treatment. “Treatment” refers to therapy, prevention and prophylaxis.

“Agent” refers to all materials that may be used to prepare pharmaceutical and diagnostic compositions, or that may be compounds, nucleic acids, polypeptides, fragments, isoforms, or other materials that may be used independently for such purposes, all in accordance with the present invention.

“Protein Isoform”, as used in the art and in one aspect of its use and meaning herein, refers to variants of a polypeptide that are encoded by the same gene, but that differ in their pI or MW, or both. Such isoforms can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation). It should be noted however, that the term “Protein Isoform” as used herein includes both the expected/wild type polypeptide and any variants thereof.

“Protein Isoform analog” refers to a polypeptide that possesses similar or identical function(s) as a Protein Isoform but need not necessarily comprise an amino acid sequence that is similar or identical to the amino acid sequence of the Protein Isoform, or possess a structure that is similar or identical to that of the Protein Isoform. As used herein, an amino acid sequence of a polypeptide is “similar” to that of a Protein Isoform if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the Protein Isoform and in respect of analogues of PIF-1 and PIF-3 will preferably be at least 70%, at least 75%, at least 80%, at least 85%, at least 90%, at least 95% or at least 99% within the double underlined region shown in FIG. 2; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the Protein Isoform and in respect of analogues of PIF-1 and PIF-3 will preferably encode at least 35 or at least 40 amino acid residues within the part of the sequence encoding the double underlined region shown in FIG. 2; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the Protein Isoform and in respect of analogues of PIF-1 and PIF-3 will preferably be at least 70%, at least 75%, at least 80%, at least 85%, at least 90%, at least 95% or at least 99% within the part of the sequence encoding the double underlined region shown in FIG. 2. As used herein, a polypeptide with “similar structure” to that of a Protein Isoform refers to a polypeptide that has a similar secondary, tertiary or quarternary structure as that of the Protein Isoform. The structure of a polypeptide can determined by methods known to those skilled in the art, including but not limited to, X-ray crystallography, nuclear magnetic resonance, and crystallographic electron microscopy.

“Protein Isoform fusion protein” refers to a polypeptide that comprises (i) an amino acid sequence of a Protein Isoform, a Protein Isoform fragment, a Protein Isoform-related polypeptide or a fragment of a Protein Isoform-related polypeptide and (ii) an amino acid sequence of a heterologous polypeptide (i.e., a non-Protein Isoform, non-Protein Isoform fragment or non-Protein Isoform-related polypeptide).

“Protein Isoform homolog” refers to a polypeptide that comprises an amino acid sequence similar to that of a Protein Isoform but does not necessarily possess a similar or identical function as the Protein Isoform.

“Protein Isoform ortholog” refers to a non-human polypeptide that (i) comprises an amino acid sequence similar to that of a Protein Isoform and (ii) possesses a similar or identical function to that of the Protein Isoform.

“Protein Isoform-related polypeptide” refers to a Protein Isoform homolog, a Protein Isoform analog, a Protein Isoform ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

“Derivative” refers to a polypeptide that comprises an amino acid sequence of a second polypeptide that has been altered by the introduction of amino acid residue substitutions, deletions or additions. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a Protein Isoform may or may not possess a functional activity of the second polypeptide.

“Fold change” includes “fold increase” and “fold decrease” and refers to the relative increase or decrease in abundance of a Protein Isoform or the relative increase or decrease in expression or activity of a polypeptide in a first sample or sample set compared to a second sample (or sample set). A Protein Isoform or polypeptide fold change may be measured by any technique known to those of skill in the art, albeit the observed increase or decrease will vary depending upon the technique used. Preferably, fold change is determined herein as described in the Examples infra.

“Modulate” in reference to expression or activity of a Protein Isoform or a Protein Isoform-related polypeptide refers to any change, e.g., upregulation or downregulation, of the expression or activity of the Protein Isoform or the Protein Isoform-related polypeptide. Those skilled in the art, based on the present disclosure, will understand that such modulation can be determined by assays known to those of skill in the art.

A “neurological disorder” is defined as a disturbance in structure or function of the central nervous system resulting from developmental abnormality, disease, injury or toxin. This includes disorders such as, for example: unipolar depression, bipolar depression (types I and II), schizophrenia, vascular dementia, multiple sclerosis and Alzheimer's disease.

“Treatment” refers to the administration of medicine or the performance of medical procedures with respect to a patient, for either prophylaxis (prevention) or to cure the infirmity or malady in the instance where the patient is afflicted.

The percent identity of two amino acid sequences or of two nucleic acid sequences can be or is generally determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbinlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97 GB3307 (published as WO 98/23950) and in U.S. Pat. No. 6,064,754, both filed Dec. 1, 1997, each of which is incorporated herein by reference in its entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

“Feature” refers to a spot detected in a 2D gel, and the term “Feature associated with a Protein Isoform of the invention” refers to a feature that is differentially present in a sample (e.g. a sample of CSF) from a subject having neurological disorder compared with a sample (e.g. a sample of CSF) from a subject free from neurological disorder. As used herein, a feature (or a Protein Isoform) is “differentially present” in a first sample with respect to a second sample when a method for detecting the feature or Protein Isoform (e.g., 2D electrophoresis or an immunoassay) gives a different signal when applied to the first and second samples. A feature or Protein Isoform is “increased” in the first sample with respect to the second if the method of detection indicates that the feature or Protein Isoform is more abundant in the first sample than in the second sample, or if the feature or Protein Isoform is detectable in the first sample and substantially undetectable in the second sample. Conversely, a feature or Protein Isoform is “decreased” in the first sample with respect to the second if the method of detection indicates that the feature or Protein Isoform is less abundant in the first sample than in the second sample or if the feature or Protein Isoform is undetectable in the first sample and detectable in the second sample.

Particularly, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); or (b) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

As used herein cerebrospinal fluid (CSF) refers to the fluid that surrounds the bulk of the central nervous system, as described in Physiological Basis of Medical Practice (J. B. West, ed., Williams and Wilkins, Baltimore, Md. 1985). CSF includes ventricular CSF and lumbar CSF.

### 5.2 Protein Isoforms of the Invention

In one aspect of the invention, two-dimensional electrophoresis is used to analyze CSF or brain tissues from a subject, preferably a living subject, in order to detect or quantify the expression of one or more Features associated with the Protein Isoforms of the invention for screening, treatment or diagnosis of neurological disorder.

In accordance with an aspect of the present invention, the Features associated with the new Protein Isoforms of the invention disclosed herein have been identified by comparing CSF and/or brain tissues samples from subjects having a neurological disorder against CSF and/or brain tissues samples from subjects free from neurological disorders. Subjects free from neurological disorders include subjects with no known disease or condition (normal subjects).

The new Protein Isoforms of the invention are all derived from the precursor protein: Pigment epithelium-derived factor (PEDF), with a SwissProt accession number: P36955 (available at http://www.expasy.org). The amino acid sequence of the precursor protein is illustrated in FIG. 1 in which the signal peptide is underlined.

The first Protein Isoform Family of the invention (hereinafter, PIF-1) corresponds to the mature protein, with the sequence illustrated in FIG. 2. All isoforms of PIF-1 are embraced i.e. all species of translation of the PEDF gene however modified by posttranslation events such as by phosphorylation or glycosylation (but not including cleavage to PIF-2 and PIF-3). This Protein Isoform Family has been identified by the Preferred Technology with a molecular weight range of 35.0 kDa to 55.8 kDa.

The second Protein Isoform Family of the invention (hereinafter, PIF-2) corresponds to the C-terminal part of the mature protein after cleavage in the Cleavage Region. The Cleavage Region is single-underlined in FIG. 2 and corresponds to a portion of the sequence rich in Lysines and Arginines, which includes a dibasic site and is thus prone to proteolytic cleavage. This Protein Isoform Family has been identified by the Preferred Technology with a molecular weight range of 18.9 kDa to 33.6 kDa.

Finally, the third Protein Isoform Family of the invention (hereinafter, PIF-3) corresponds to the N-terminal part of the mature protein after cleavage in the Cleavage Region. This Protein Isoform Family has been identified by the Preferred Technology with a molecular weight in the range of 13.9 kDa.

PIF-2 and PIF-3 are novel protein isoforms which are claimed per se, together with antibodies (or other affinity reagents such as Affibodies) which bind immunospecifically thereto.

Thus we claim a protein having the amino acid sequence corresponding to FIG. 2 having as its N terminus a residue within the underlined region (i.e. NL . . . IK) and finishing at the C-terminus of FIG. 2.

We also claim similar proteins which have at least 70, or 75, or 80, or 85, or 90, or 95 or 99% sequence identity to such a protein and which share the same function.

We also claim a protein having the amino acid sequence corresponding to FIG. 2 starting at the N terminus and having at its C terminus a residue within the underlined region (i.e. NL . . . IK).

We also claim similar proteins which have at least 40, or 45, or 50, or 55, or 60, or 65, or 70, or 75, or 80, or 85, or 90, or 95 or 99% sequence identity to such a protein sequence and which within the double underline region of FIG. 3 has at least 70, or 75, or 80, or 85, or 90, or 95 or 99% sequence identity with the sequence of FIG. 2 and which share the same function.

We also claim nucleic acid molecules (RNA and DNA) which encode for the aforementioned proteins, vectors containing such nucleic acids and transformed host cells containing such vectors or nucleic acid molecules

Thus Protein Isoform Family PIF-1 is suitably defined by proteins of sequence corresponding to that shown in FIG. 2, and proteins having substantial identity thereto, including all isoforms thereof.

Proteins having substantial identity thereto will typically have at least 90% sequence identity to said protein overall and at least 95% sequence identity within the active site region (amino acids 59-102 in FIG. 2)

Thus Protein Isoform Family PIF-2 is suitably defined by proteins of sequence corresponding to the C-terminus of the protein shown in FIG. 2 having as its N terminus a residue between residues 116 and 132, and proteins having substantial identity thereto, including all isoforms thereof.

Proteins having substantial identity thereto will typically have at least 90% sequence identity to said protein overall.

Thus Protein Isoform Family PIF-3 is suitably defined by proteins of sequence corresponding to the N-terminus of the protein shown in FIG. 2 having as its C terminus a residue between residues 116 and 132, and proteins having substantial identity thereto, including all isoforms thereof.

Proteins having substantial identity thereto will typically have at least 90% sequence identity to said protein overall and at least 95% sequence identity within the active site region (amino acids 59 to 102 in FIG. 2) or such portion of it in the protein.

Antibodies (and other affinity reagents such as Afibodies) which bind immunospecifically to PIF-1 do not bind significantly to other relevant proteins, especially PIF-2 or PIF-3. Antibodies which bind immunospecifically to PIF-2 do not bind significantly to other relevant proteins, especially PIF-1 or PIF-3. Antibodies which bind immunospecifically to PIF-3 do not bind significantly to other relevant proteins, especially PIF-1 or PIF-2. In this context “relevant proteins” are proteins from which it will be desirable to discriminate by antibody minding. “Do not bind significantly” maans, for example, that the binding strength to the target is at least 10, 100, 1000 or preferably at least 10000 time stronger than the binding strength to non-desired targets.

The abundance of the new Protein Isoforms of the invention have surprisingly been found to be correlated with the health state of normal controls and neurological disorder patients, in particular disorders associated with de-regulated (eg overactivation of) glutamate signalling. For example, FIG. 3 illustrates the relative abundancies of the 3 Protein Isoform Families of the invention in the CSF of normal controls and of Schizophrenia patients. A general decrease of PIF-1 can be observed in disease, together with an increase of PIF-2 and PIF-3 in disease. This is indicative of a processing of the PIF-1 isoforms into the 2 smaller PIF-2 and PIF-3 isoforms in the context of the disease.

This increased proteolytic processing has been equally observed in samples from Alzheimer's Disease, Depression, Multiple Sclerosis and Vascular Dementia patients, and is illustrated in FIG. 4 along with expression in other CNS diseases.

In addition, FIG. 2 illustrates the PEDF domain (amino acids in bold and double-underlined) which has been shown to have neurotrophic and neuroprotective activity (see for example, Bilak M. et al, J. Neurosci., Nov. 1, 2002, 22(21):9378-9386; Alberdi E. et al, The Journal of Biological Chemistry, 1999, Vol. 274, No. 44, pp. 31605-31612). This domain thus appears to belong, in addition to the mature forms of the PIF-1 family members, to the PIF-3 Protein Isoform Family of the invention.

Moreover, the PEDF protein is reported to be the site of a number of Single Nucleotide Polymorphisms associated with diseases. For example, the Met72 Thr polymorphism (numbering referenced to the sequence of FIG. 1) has been reported to be associated with age-related macular degeneration (Yamagishi S. et al., Med. Hypotheses., 2005; 64(6):1202-4). In addition, decreased expression of PEDF is known to be involved in Diabetic Nephropathy (Joshua J. et al., Diabetes 54:243-250, 2005; Boehm B O. et al., Diabetologia. 2003 March; 46(3):394-400). Interestingly, the Cleavage Region defined above (and single-underlined in FIG. 2) is located very near a reported SNP at position 132 (Pro->Arg or Pro->His, at position 113 in the sequence of FIG. 2 since this Figure represents the mature sequence). Proline are amino acids with a strong structural implication, and the mutation to Arg and even His is very likely to disrupt the secondary and tertiary structure of the wild type protein such as to create a protease processing site. The occurrence of PIF-2 and PIF-3 from proteolytic processing of PIF-1 in the context of disease would thus have a genetic susceptibility in the form of the SNP variation at position 132.

Thus according to the invention we provide a method for screening for or diagnosis or prognosis of a neurological disorder associated with de-regulated glutamate signalling in a subject, for determining the stage or severity of such a neurological disorder in a subject, for identifying a subject at risk of developing such a neurological disorder, or for monitoring the effect of therapy administered to a subject having such a neurological disorder, said method comprising:

(a) analyzing a test sample of body fluid or tissue from the subject said sample comprising at least one Protein Isoform selected from the following Protein Isoform Families: PIF-1, PIF-2, and PIF-3 in a detectable amount; and

(b) comparing the abundance of said Protein Isoform(s) in the test sample or the abundance of said Protein Isoform(s) relative to another Protein Isoform with the abundance or relative abundance of said Protein Isoform(s) in a test sample from one or more persons free from neurological disorder, or with a previously determined reference range for that Protein Isoform in subjects free from neurological disorder, wherein a diagnosis of or a positive result in screening for or a prognosis of a more advanced condition of said neurological disorder is indicated by (i) decreased abundance or relative abundance of PIF-1 and/or (ii) increased abundance or relative abundance of PIF-2 and/or (iii) decreased abundance or relative abundance of PIF-3.

Typically a diagnosis of or a positive result in screening for or a prognosis of a more advanced condition of said neurological disorder is indicated by (i) decreased abundance of PIF-1 and/or (ii) increased abundance of PIF-2. Suitably it is indicated by (i) decreased abundance of PIF-1 and (ii) increased abundance of PIF-2. Suitably it is also indicated by increased abundance of PIF-3.

In a preferred embodiment, the Protein Isoforms of the PEF-2 family are defined as polypeptides consisting of substantially all the amino acid sequence from the beginning of the cleavage site in FIG. 2 (starting at: NLKSASR . . . ) to the C-terminal part of the mature protein ( . . . ILDPRGP). One skilled in the art will recognize that the members of the PIF-2 Protein Isoform family will differ among each other by variations in the length of their amino acid sequence (for example with different starting positions within the Cleavage Region of FIG. 2), and by the extent and the number of their post-translational modifications. These post-translational modifications may include, as examples and by no way of limitation, phosphorylation on Threonine 219 (the numbering refers to the sequence of FIG. 2), phosphorylation on Serine 226, phosphorylation on Serine 301, and phosphorylation on Tyrosine 302. In addition, a detailed protocol suitable to investigate glycosylation sites in Protein Isoforms is presented in Section 7.

In yet a preferred embodiment, the Protein Isoforms of the PIF-2 family contain one or more of the following sequences:

| | |
|---|---|
| IVFEKK | (SEQ ID No 16) |
| SSFVAPLEK | (SEQ ID No 30) |
| SYGTRPRVLTGNPR | (SEQ ID No 34) |
| LDLQEINNWVQAQMK | (SEQ ID No 19) |
| EIPDEISILLLGVAH | (SEQ ID No 8) |
| FKGQWVTK | (SEQ ID No 12) |
| KTSLEDFYLDEER | (SEQ ID No 17) |
| VPMMSDPK | (SEQ ID No 41) |
| YGLDSDLSCKIAQLPLTGSMSIIFFLPLK | (SEQ ID No 45) |
| VTQNLTLIEESLTSEFIHDIDR | (SEQ ID No 43) |
| TVQAVLTVPK | (SEQ ID No 38) |
| LSYEGEVTK | (SEQ ID No 25) |
| SLQEMK | (SEQ ID No 29) |
| LQSLFDSPDFSK | (SEQ ID No 24) |
| ITGKPIK | (SEQ ID No 15) |
| LTQVEHR | (SEQ ID No 28) |
| DTDTGALLFIGK | (SEQ ID No 7) |

In an even preferred embodiment, the Protein Isoforms of the PIF-2 family do not contain the following sequences:

| | |
|---|---|
| LAAAVSNFGYDLYR | (SEQ ID No 18) |
| SSTSPTTNVLLSPLSVATALSALSLGAEQR | (SEQ ID No 31) |

or the following sequence:

| | |
|---|---|
| SSMSPTTNVLLSPLSVATALSALSLGAEQR | (SEQ ID No 56) |
| TESIIHR | (SEQ ID No 35) |
| ALYYDLISSPDIHGTYK | (SEQ ID No 1) |
| ELLDTVTAPQK | (SEQ ID No 9) |

In a preferred embodiment, the Protein Isoforms of the PIF-3 family are defined as polypeptides consisting of substantially all the amino acid sequence from the beginning of the mature protein in FIG. 2 (starting at: QNPASPPE . . . ) to end of the Cleavage Region of FIG. 2 ( . . . IVFEKKLRIK). One skilled in the art will recognize that the members of the PIF-3 Protein Isoform family will differ among each other by variations in the length of their amino acid sequence (for example with different ending positions within the Cleavage Region of FIG. 2), and by the extent and the number of their post-translational modifications. These post-translational modifications may include, as examples and by no way of limitation, Phosphorylation on Serine 95. In addition, a detailed protocol suitable to investigate glycosylation sites in Protein Isoforms is presented in Section 7.

In yet a preferred embodiment, the Protein Isoforms of the PIF-3 family contain one or more of the following sequences:

| | |
|---|---|
| LAAAVSNFGYDLYR | (SEQ ID No 18) |
| SSTSPTTNVLLSPLSVATALSALSLGAEQR | (SEQ ID No 31) |

or the following sequence:

| | |
|---|---|
| SSMSPTTNVLLSPLSVATALSALSLGAEQR | (SEQ ID No 56) |
| TESIIHR | (SEQ ID No 35) |
| ALYYDLISSPDTHGTYK | (SEQ ID No 1) |
| ELLDTVTAPQK | (SEQ ID No 9) |
| IVFEKK | (SEQ ID No 16) |

In an even preferred embodiment, the Protein Isoforms of the PIF-3 family do not contain the following sequences:

| | |
|---|---|
| SSFVAPLEK | (SEQ ID No 30) |
| SYGTRPRVLTGNPR | (SEQ ID No 34) |
| LDLQEINNWVQAQMK | (SEQ ID No 19) |
| EIPDEISILLLGVAH | (SEQ ID No 8) |
| FKGQWVTK | (SEQ ID No 12) |
| KTSLEDFYLDEER | (SEQ ID No 17) |
| VPMMSDPK | (SEQ ID No 41) |
| YGLDSDLSCKIAQLPLTGSMSIIFFLPLK | (SEQ ID No 45) |
| VTQNLTLIEESLTSEFIHDIDR | (SEQ ID No 43) |
| TVQAVLTVPK | (SEQ ID No 38) |
| LSYEGEVTK | (SEQ ID No 25) |
| SLQEMK | (SEQ ID No 29) |
| LQSLFDSPDFSK | (SEQ ID No 24) |
| ITGKPIK | (SEQ ID No 15) |
| LTQVEHR | (SEQ ID No 28) |
| DTDTGALLFIGK | (SEQ ID No 7) |

Thus in a method, kit, affinity reagent or preparation according to the invention the Protein Isoform Family PIF-2 may suitably be defined by proteins comprising one or more sequences selected from SEQ ID Nos 16, 30, 34, 19, 8, 12, 17, 41, 45, 43, 38, 25, 29, 24, 15, 28 and 7.
Particularly, in a method, kit, affinity reagent or preparation according to the invention the Protein Isoform Family PIF-2 may be further defined by proteins not comprising a sequence selected from SEQ ID Nos 18, 31 (or more preferably 56), 35, 1 and 9.
Thus in a method, kit, affinity reagent or preparation according to the invention the Protein Isoform Family PIF-3 may suitably be defined by proteins comprising one or more sequence selected from SEQ ID Nos 18, 31 (or more preferably 56), 35, 1, 9 and 16.
Particularly, in a method, kit, affinity reagent or preparation according to the invention the Protein Isoform Family PIF-2 may be further defined by proteins not comprising a sequence selected from SEQ ID Nos 30, 34, 19, 8, 12, 17, 41, 45, 43, 38, 25, 29, 24, 15, 28 and 7.
An exemplary amino acid sequences for protein isoforms of Protein Isoform Family PIF-2 may comprise the sequence of SEQ ID No 54. For example it may have the sequence of SEQ ID No 55.
An exemplary amino acid sequences for protein isoforms of Protein Isoform Family PIF-3 may comprise the sequence of SEQ ID No 52. For example it may have the sequence of SEQ ID No 53.
Table I below illustrates some representatives of each of the Three PIF families, which were identified in Cerebro Spinal Fluid.

TABLE I
Protein Isoforms Expression in CSF in Alzheimer's disease (AD),
Schizophrenia (Schiz), Multiple Sclerosis (MS), Vascular Dementia (VDM)
and Bipolar Depression (BP).

| Isoform | PIF | | | | | | | | |
|---|---|---|---|---|---|---|---|---|---|
| # | # | pI | MW | Tryptic Sequences | AD | Schiz | MS | VDM | BP |
| 1 | 1 | 5.42 | 55768 | KTSLEDFYLDEER, LAAAVSNFGYDLYR, | | | | | |
| | | | | LTQVEHR, TESLLHR, TSLEDFYLDEER | | | | | |
| 2 | 1 | 5.24 | 54713 | ALYYDLLSSPDLHGTYK, CGALQGAVGNK, | | | | | |
| | | | | DLLASVTAPQK, ELLDTVTAPQK, | | | | | |
| | | | | KTSLEDFYLDEER, LAAAVSNFGYDLYR, | | | | | |
| | | | | LSYEGEVTK, LTQVEHR, SSFVAPLEK, | | | | | |
| | | | | TESLLHR, TSLEDFYLDEER, VLTGNPR | | | | | |
| 3 | 1 | 5.16 | 52996 | KTSLEDFYLDEER | | | | | |
| 4 | 1 | 5.41 | 52672 | GQWVTK, KTSLEDFYLDEER, | | | | | |
| | | | | LAAAVSNFGYDLYR, LQLCGTSGK, | | | | | |
| | | | | LQSLFDSPDFSK, LTQVEHR, SSFVAPLEK, | | | | | |
| | | | | TESLLHR, TSLEDFYLDEER, VLTGNPR, | | | | | |
| | | | | YGLDSDLSCK | | | | | |
| 5 | 1 | 5.03 | 51527 | ALYYDLLSSPDLHGTYK, KTSLEDFYLDEER, | | | | | Down |
| | | | | LAAAVSNFGYDLYR, LQSLFDSPDFSK, | | | | | |
| | | | | LTQVEHR, SSFVAPLEK, TESLLHR, | | | | | |
| | | | | TSLEDFYLDEER, VLTGNPR | | | | | |
| 6 | 1 | 5.30 | 51314 | ALYYDLLSSPDLHGTYK, DTDTGALLFLGK, | | | | | |
| | | | | ELLDTVTAPQK, GQWVTK, | | | | | |
| | | | | KTSLEDFYLDEER, LAAAVSNFGYDLYR, | | | | | |
| | | | | LQSLFDSPDFSK, LSYEGEVTK, LTQVEHR, | | | | | |
| | | | | SSFVAPLEK, SYGTRPR, TESLLHR, | | | | | |
| | | | | TSLEDFYLDEER, VLTGNPR, YGLDSDLSCK | | | | | |
| 7 | 1 | 5.11 | 51192 | ALYYDLLSSPDLHGTYK, KTSLEDFYLDEER, | Down | Down | Down | Down | |
| | | | | LAAAVSNFGYDLYR, LTQVEHR, SSFVAPLEK, | | | | | |
| | | | | SYGTRPR, TESLLHR, TSLEDFYLDEER, | | | | | |
| | | | | VLTGNPR | | | | | |
| 8 | 1 | 5.19 | 50477 | ALYYDLLSSPDLHGTYK, | Down | Down | Down | Down | |
| | | | | ELPDELSLLLLGVAHFK, GQWVTK, | | | | | |
| | | | | KTSLEDFYLDEER, LAAAVSNFGYDLYR, | | | | | |
| | | | | LQSLFDSPDFSK, LSYEGEVTK, LTQVEHR, | | | | | |
| | | | | SSFVAPLEK, SYGTRPR, TESLLHR, | | | | | |
| | | | | TSLEDFYLDEER, TVQAVLTVPK, VLTGNPR, | | | | | |
| | | | | VPMMSDPK, YGLDSDLSCK | | | | | |
| 9 | 1 | 5.31 | 50056 | ALYYDLLSSPDLHGTYK, DTDTGALLFLGK, | Down | Down | Down | Down | |
| | | | | ELLDTVTAPQK, ELPDELSLLLLGVAHFK, | | | | | |
| | | | | GQWVTK, KTSLEDFYLDEER, | | | | | |
| | | | | LAAAVSNFGYDLYR, LQSLFDSPDFSK, | | | | | |
| | | | | LSYEGEVTK, LTGKPLK, LTQVEHR, | | | | | |
| | | | | SSFVAPLEK, SYGTRPR, TESLLHR, | | | | | |
| | | | | TSLEDFYLDEER, VLTGNPR, YGLDSDLSCK | | | | | |
| 10 | 1 | 5.42 | 50004 | ALYYDLLSSPDLHGTYK, DTDTGALLFLGK, | Down | Down | Down | Down | |
| | | | | ELLDTVTAPQK, ELPDELSLLLLGVAHFK, | | | | | |
| | | | | GQWVTK, KTSLEDFYLDEER, | | | | | |
| | | | | LAAAVSNFGYDLYR, LLTGNSR, | | | | | |
| | | | | LQLCGTSGK, LQSLFDSPDFSK, LSYEGEVTK, | | | | | |
| | | | | LTFPLDYHLNQPFLFVLR, LTQVEHR, | | | | | |
| | | | | SSFVAPLEK, SYGTRPR, TESLLHR, | | | | | |
| | | | | TSLEDFYLDEER, TVQAVLTVPK, | | | | | |
| | | | | VHLCESLNSNPR, VLTGNPR, VPMMSDPK, | | | | | |
| | | | | YGLDSDLSCK | | | | | |
| 11 | 1 | 5.50 | 49028 | ALYYDLLSSPDLHGTYK, GQWVTK, | | | | | |
| | | | | KTSLEDFYLDEER, LAAAVSNFGYDLYR, | | | | | |
| | | | | LQSLFDSPDFSK, LTQVEHR, SSFVAPLEK, | | | | | |
| | | | | SYGTRPR, TESLLHR, TSLEDFYLDEER, | | | | | |
| | | | | VLTGNPR, YGLDSDLSCK | | | | | |
| 12 | 1 | 5.53 | 48948 | ALYYDLLSSPDLHGTYK, ELLDTVTAPQK, | | Down | Down | Down | Down |
| | | | | GQWVTK, KTSLEDFYLDEER, | | | | | |
| | | | | LAAAVSNFGYDLYR, LQSLFDSPDFSK, | | | | | |
| | | | | LSYEGEVTK, LTQVEHR, SSFVAPLEK, | | | | | |
| | | | | SYGTRPR, TESLLHR, TSLEDFYLDEER, | | | | | |
| | | | | VLTGNPR, YGLDSDLSCK | | | | | |
| 13 | 1 | 5.31 | 48848 | ALYYDLLSSPDLHGTYK, DTDTGALLFLGK, | | | | | |
| | | | | ELLDTVTAPQK, ELLDTVTAR, GQWVTK, | | | | | |
| | | | | KTSLEDFYLDEER, LAAAVSNFGYDLYR, | | | | | |
| | | | | LQSLFDSPDFSK, LSYEGEVTK, LTQVEHR, | | | | | |
| | | | | SSFVAPLEK, SYGTRPR, TESLLHR, | | | | | |
| | | | | TSLEDFYLDEER, TVQAVLTVPK, VLTGNPR, | | | | | |
| | | | | YGLDSDLSCK | | | | | |
| 14 | 1 | 5.75 | 48508 | ALYYDLLSSPDLHGTYK, KTSLEDFYLDEER, | | | | | |
| | | | | LAAAVSNFGYDLYR, LTQVEHR, TESLLHR, | | | | | |
| | | | | TSLEDFYLDEER, VLTGNPR | | | | | |
| 15 | 1 | 5.63 | 48347 | KTSLEDFYLDEER, LAAAVSNFGYDLYR, | | | | | |
| | | | | LTQVEHR, SSFVAPLEK, TESLLHR, | | | | | |
| | | | | TSLEDFYLDEER, VLTGNPR | | | | | |
| 16 | 1 | 5.67 | 48051 | ALYYDLLSSPDLHGTYK, DTDTGALLFLGK, | Down | Down | Down | Down | Down |
| | | | | ELLDTVTAPQK, ELPDELSLLLLGVAHFK, | | | | | |
| | | | | GPLWPLR, GQWVTK, KTSLEDFYLDEER, | | | | | |
| | | | | LAAAVSNFGYDLYR, LQSLFDSPDFSK, | | | | | |
| | | | | LSYEGEVTK, LTQVEHR, SSFVAPLEK, | | | | | |
| | | | | SYGTRPR, TESLLHR, TSLEDFYLDEER, | | | | | |
| | | | | TVQAVLTVPK, VLTGNPR, VPMMSDPK, | | | | | |
| | | | | YGLDSDLSCK | | | | | |
| 17 | 1 | 5.52 | 47931 | KTSLEDFYLDEER, LAAAVSNFGYDLYR, | | | | | |
| | | | | LQSLFDSPDFSK, LTQVEHR, SSFVAPLEK, | | | | | |
| | | | | SYGTRPR, TESLLHR, TSLEDFYLDEER, | | | | | |
| | | | | VLTGNPR | | | | | |
| 18 | 1 | 5.83 | 47846 | ALYYDLLSSPDLHGTYK, KTSLEDFYLDEER, | | Down | Down | Down | Down |
| | | | | LAAAVSNFGYDLYR, LQSLFDSPDFSK, | | | | | |
| | | | | LTFPLDYHLNQPFLFVLR, LTQVEHR | | | | | |
| | | | | SSFVAPLEK, SYGTRPR, TESLLHR, | | | | | |
| | | | | TSLEDFYLDEER, VLTGNPR, VPMMSDPK | | | | | |
| 19 | 1 | 6.04 | 47247 | ALYYDLLSSPDLHGTYK, KTSLEDFYLDEER, | Down | Down | Down | Down | |
| | | | | LAAAVSNFGYDLYR, LQSLFDSPDFSK, | | | | | |
| | | | | LTQVEHR, SSFVAPLEK, SYGTRPR, | | | | | |
| | | | | TESLLHR, TSLEDFYLDEER, VLTGNPR, | | | | | |
| | | | | YGLDSDLSCK | | | | | |
| 20 | 2 | 6.38 | 34273 | KTSLEDFYLDEER, LQSLFDSPDFSK, | | Up | | | |
| | | | | LSYEGEVTK, LTQVEHR, SSFVAPLEK, | | | | | |
| | | | | SYGTRPR, TSLEDFYLDEER, VLTGNPR, | | | | | |
| | | | | VPMMSDPK, YGLDSDLSCK | | | | | |
| 21 | 2 | 5.39 | 34112 | GQWVTK, KTSLEDFYLDEER, | | | | | |
| | | | | LQSLFDSPDFSK, SSFVAPLEK, SYGTRPR, | | | | | |
| | | | | TSLEDFYLDEER, VLTGNPR | | | | | |
| 22 | 2 | 6.64 | 33736 | KTSLEDFYLDEER, LTQVEHR, | | | | | |
| | | | | TSLEDFYLDEER, VLTGNPR | | | | | |
| 23 | 2 | 6.72 | 33490 | DTDTGALLFLGK, KTSLEDFYLDEER, | | | | | |
| | | | | LSYEGEVTK, LTQVEHR, SSFVAPLEK, | | | | | |
| | | | | TSLEDFYLDEER, VLTGNPR | | | | | |
| 24 | 2 | 6.10 | 33206 | TSLEDFYLDEER | | | Up | | |
| 25 | 2 | 7.12 | 32018 | DQQLGAGADER, KTSLEDFYLDEER, | | | | | |
| | | | | LQSLFDSPDFSK, LTQVEHR, | | | | | |
| | | | | TSLEDFYLDEER, VLTGNPR, VPMMSDPK | | | | | |
| 26 | 2 | 5.61 | 31612 | KTSLEDFYLDEER, LQSLFDSPDFSK, | Up | | | | |
| | | | | LTQVEHR, SLQEMK, SSFVAPLEK, | | | | | |
| | | | | SYGTRPR, TSLEDFYLDEER, VLTGNPR, | | | | | |
| | | | | VPMMSDPQAVLR | | | | | |
| 27 | 2 | 5.81 | 31129 | KTSLEDFYLDEER, LQSLFDSPDFSK, | | Up | | | |
| | | | | LTQVEHR, TSLEDFYLDEER, VLTGNPR | | | | | |
| 28 | 2 | 6.12 | 31072 | DTDTGALLFLGK, GQWVTK, | Up | | | | |
| | | | | KTSLEDFYLDEER, LSYEGEVTK, LTQVEHR, | | | | | |
| | | | | SSFVAPLEK, SYGTRPR, TSLEDFYLDEER, | | | | | |
| | | | | TVQAVLTVPK, VLTGNPR | | | | | |
| 29 | 2 | 6.08 | 30621 | LTQVEHR, TSLEDFYLDEER, VLTGNPR | | | | | |
| 30 | 2 | 6.38 | 29891 | KTSLEDFYLDEER, LQSLFDSPDFSK, | Up | Up | | Up | |
| | | | | LTQVEHR, SSFVAPLEK, TSLEDFYLDEER, | | | | | |
| | | | | VLTGNPR | | | | | |
| 31 | 2 | 5.75 | 29790 | KTSLEDFYLDEER, LDLQELNNWVQAQMK, | | | | | |
| | | | | LQSLFDSPDFSK, LTQVEHR, SSFVAPLEK, | | | | | |
| | | | | SYGTRPR, TSLEDFYLDEER, VLTGNPR | | | | | |
| 32 | 2 | 5.37 | 29417 | KTSLEDFYLDEER, LTQVEHR, | | | | | |
| | | | | TSLEDFYLDEER | | | | | |
| 33 | 2 | 5.24 | 28017 | KTSLEDFYLDEER, LTQVEHR, | | | | Up | |
| | | | | TSLEDFYLDEER | | | | | |
| 34 | 3 | 6.67 | 19433 | LAAAVSNFGYDLYR, LLTGNSR, LPWPPR, | | | | | |
| | | | | SSFVAPLEK, SVQELK, SYGTRPR, | | | | | |
| | | | | TESLLHR, VLTGNPR | | | | | |
| 35 | 3 | 5.57 | 14498 | LAAAVSNFGYDLYR, TESLLHR | Up | Up | | Up | |
| 36 | 3 | 4.47 | 13889 | ALYYDLLSSPDLHGTYK, ELLDTVTAPQK, | | | | | |
| | | | | LAAAVSNFGYDLYR, TESLLHR | | | | | |

Thus in a method, kit, affinity reagent or preparation according to the invention the Protein Isoform Family PIF-1 may suitably be defined by protein isoforms having a pI and MW as shown in Table 1 rows 1-19. Suitably said proteins comprise one or more sequences recited in the column headed “tryptic sequences”.
Thus in a method, kit, affinity reagent or preparation according to the invention the Protein Isoform Family PIF-2 may suitably be defined by protein isoforms having a pI and MW as shown in Table 1 rows 20-33. Suitably said proteins comprise one or more sequences recited in the column headed “tryptic sequences”.
Thus in a method, kit, affinity reagent or preparation according to the invention the Protein Isoform Family PIF-3 may suitably be defined by protein isoforms having a pI and MW as shown in Table 1 rows 34-36. Suitably said proteins comprise one or more sequences recited in the column headed “tryptic sequences”.
Exemplary isoforms are shown in the Figures. For example Isoform 12 in family PIF-1 is associated with schizophrenia by virtue of its lower abundance. Similar deductions can be made for other isoforms shown in the Figures and Table 1.

For any given Protein Isoform, the signal obtained upon analyzing a sample from subjects having a neurological disorder relative to the signal obtained upon analyzing the same sample from subjects free from the neurological disorder will depend upon the particular analytical protocol and detection technique that is used. Accordingly, those skilled in the art will understand that any laboratory, based on the present description, can establish a suitable reference range for any Protein Isoform in subjects free from neurological disorder according to the analytical protocol and detection technique in use. In particular, at least one positive control Protein Isoform sample from a subject known to have neurological disorder or at least one negative control Protein Isoform sample from a subject known to be free from neurological disorder (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no substantial discernable protein feature.

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight in Daltons and the apparent isoelectric point of a feature or protein isoform as measured in careful accordance with the Reference Protocol identified in Section 6 below. When the Reference Protocol is followed and when samples are run in duplicate or a higher number of replicates, variation in the measured mean pI of a Protein Isoform is typically less than 3% and variation in the measured mean MW of a Protein Isoform is typically less than 5%. Where the skilled artisan wishes to diverge from the Reference Protocol, calibration experiments should be performed to compare the MW and pI for each Protein Isoform as detected (a) by the Reference Protocol and (b) by the divergent.

The Protein Isoforms of the invention can be used, for example, for detection, treatment, diagnosis, or the drug development or pharmaceutical products. In one embodiment of the invention, CSF or a brain biopsy from a subject (e.g., a subject suspected of having neurological disorder) is analyzed by 2D electrophoresis for quantitative detection of a PIF-1 family member. A decreased abundance of a PIF-1 family member in the CSF or brain biopsy from the subject relative to CSF or brain biopsy from a subject or subjects free from neurological disorder (e.g., a control sample or a previously determined reference range) indicates the presence of neurological disorder.

In another embodiment of the invention, CSF or a brain biopsy from a subject is analyzed by 2D electrophoresis for quantitative detection of a PIF-2 and/or a PIF-3 family member. An increased abundance of said PIF-2 and/or a PIF-3 family member in the CSF or brain biopsy from the subject relative to CSF or brain biopsy from a subject or subjects free from neurological disorder (e.g., a control sample or a previously determined reference range) indicates the presence of neurological disorder.

In yet another embodiment, CSF or a brain biopsy from a subject is analyzed by 2D electrophoresis for quantitative detection of (a) a PIF-1 family member, whose decreased abundance indicates the presence of neurological disorder; and (b) a PIF-2 and/or a PIF-3 family member, whose increased abundance indicates the presence of neurological disorder.

In yet another embodiment of the invention, CSF or a brain biopsy from a subject is analyzed by 2D electrophoresis for quantitative detection of a PIF-1 family member, and/or a PIF-2 and/or a PIF-3 family member wherein the ratio of the one or more Protein Isoform(s) relative to another Protein Isoform indicates that neurological disorder is present. In a specific embodiment, a decrease in a PIF-1 family member/a PIF-2 and/or a PIF-3 family member ratio in a sample being tested relative to the same PIF-1 family member/PIF-2 and/or PIF-3 family member ratio in a control sample or a reference range indicates the presence of neurological disorder. In another specific embodiment, one may measure one or more Protein Isoforms from the PIF-2 and/or PIF-3 families in a test sample, and compare them to a PIF-1 family member, as a method for detecting the presence of neurological disorder. Thus, an increase in one or more PIF-2 and/or PIF-3 family member/PIF-1 family member ratios in a test sample relative to the PIF-2 and/or PIF-3 family member/PIF-1 family member ratios in a control sample or a reference range indicates the presence of neurological disorder.

In a preferred embodiment, CSF or a brain biopsy from a subject is analyzed for quantitative detection of a plurality of Protein Isoforms.

As shown above, the Protein Isoforms include isoforms of known proteins where the isoforms were not previously known to be associated with neurological disorder. For each Protein Isoform, the present invention additionally provides: (a) a preparation comprising the isolated Protein Isoform; (b) a preparation comprising one or more fragments of a Protein Isoform; and (c) antibodies (or other affinity reagents eg Affibodies) that bind to said Protein Isoform, to said fragments, or both to said Protein Isoform and to said fragments. As used herein, a Protein Isoform is “isolated” when it is present in a preparation that is substantially free of other proteins, i.e., a preparation in which less than 10% (particularly less than 5%, more particularly less than 1%) of the total protein present is contaminating protein(s). Another protein is a protein or protein isoform having a significantly different pI or MW from those of the isolated Protein Isoform, as determined by 2D electrophoresis. As used herein, a “significantly different” pI or MW is one that permits the other protein to be resolved from the Protein Isoform on 2D electrophoresis, performed according to the Reference Protocol.

In one embodiment, an isolated protein is provided, that comprises a peptide with the amino acid sequence identified in Table I for a Protein Isoform, said protein having a pI and MW within 10% (particularly within 5%, more particularly within 1%) of the values identified in Table I for that Protein Isoform.

The Protein Isoforms of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the Protein Isoforms are separated on a 2-D gel by virtue of their MWs and pIs and are visualized by staining the gel. In one embodiment, the Protein Isoforms are stained with a fluorescent dye and imaged with a fluorescence scanner. Sypro Red (Molecular Probes, Inc., Eugene, Oreg.) is a suitable dye for this purpose. Alternative dyes are described in U.S. Ser. No. 09/412,168, filed Oct. 5, 1999, and incorporated herein by reference in its entirety.

Alternatively, Protein Isoforms can be detected in an immunoassay. In one embodiment, an immunoassay is performed by contacting a sample with an anti-Protein Isoform antibody (or other affinity reagent such as an Affibody) under conditions such that immunospecific binding can occur if the Protein Isoform is present, and detecting or measuring the amount of any immunospecific binding by the antibody. Anti-Protein Isoform antibodies can be produced by the methods and techniques described herein; examples of such antibodies known in the art are set forth in Table H. These antibodies shown in Table II are already known to bind to the protein of which the Protein Isoform is itself a family member. Particularly, the anti-Protein Isoform antibody preferentially binds to the Protein Isoform rather than to other isoforms of the same protein. In a particular embodiment, the anti-Protein Isoform antibody binds to the Protein Isoform with at least 2-fold greater affinity, more particularly at least 5-fold greater affinity, still more particularly at least 10-fold greater affinity, than to said other isoforms of the same protein. When the antibodies shown in Table II do not display the required preferential selectivity for the target Protein Isoform, one skilled in the art can generate additional antibodies by using the Protein Isoform itself for the generation of such antibodies.

Protein Isoforms can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-Protein Isoform antibodies as described herein, e.g., the antibodies identified in Table H, or others raised against the Protein Isoforms of interest as those skilled in the art will appreciate based on the present description. The immunoblots can be used to identify those anti-Protein Isoform antibodies displaying the selectivity required to immuno-specifically differentiate a Protein Isoform from other isoforms encoded by the same gene.

TABLE II
Table II: Known Antibodies That Recognize Protein
Isoforms or Protein Isoforms-Related Polypeptides

| PIF-# | Antibody | Manufacturer | Catalogue number |
|---|---|---|---|
| PIF-1 | SERPINF1 antibody | Abcam Ltd | ab14993 |
| PIF-2 | Pigment-Epithelium | CHEMICON | MAB1059 |
| | Derived Factor | International, | |
| | [PEDF] (Hu) | Inc. | |

**Further Information about these antibodies can be obtained from their commercial sources at: Chemicon International, Inc - http://www.chemicon.com/, Abcam Ltd; - www.abcam.com..

In one embodiment, binding of antibody in tissue sections can be used to detect Protein Isoform localization or the level of one or more Protein Isoforms. In a specific embodiment, antibody to a Protein Isoform can be used to assay a tissue sample (e.g., a brain biopsy) from a subject for the level of the Protein Isoform where a substantially changed level of Protein Isoform is indicative of neurological disorder. As used herein, a “substantially changed level” means a level that is increased or decreased compared with the level in a subject free from neurological disorder or a reference level. If desired, the comparison can be performed with a matched sample from the same subject, taken from a portion of the body not affected by the neurological disorder.

Any suitable immunoassay can be used to detect a Protein Isoform, including, without limitation, competitive and non-competitive assay systems using techniques such as western blots, radioimmunoassays, ELISAs (enzyme linked immunosorbent assays), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a Protein Isoform can be detected in a fluid sample (e.g., CSF, blood, urine, or tissue homogenate) by means of a two-step sandwich assay. In the first step, a capture reagent (e.g., an anti-Protein Isoform antibody) is used to capture the Protein Isoform. Examples of such antibodies known in the art are set forth in Table II. The capture reagent can optionally be immobilized on a solid phase. In the second step, a directly or indirectly labeled detection reagent is used to detect the captured Protein Isoform. In one embodiment, the detection reagent is a lectin. A lectin can be used for this purpose that preferentially binds to the Protein Isoform rather than to other isoforms that have the same core protein as the Protein Isoform or to other proteins that share the antigenic determinant recognized by the antibody. In a preferred embodiment, the chosen lectin binds to the Protein Isoform with at least 2-fold greater affinity, more preferably at least 5-fold greater affinity, still more preferably at least 10-fold greater affinity, than to said other isoforms that have the same core protein as the Protein Isoform or to said other proteins that share the antigenic determinant recognized by the antibody. Based on the present description, a lectin that is suitable for detecting a given Protein Isoform can readily be identified by those skilled in the art using methods well known in the art, for instance upon testing one or more lectins enumerated in Table I on pages 158-159 of Sumar et al. Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the Protein Isoform in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding a Protein Isoform, a related gene (e.g. a gene having sequence homology), or related nucleic acid sequences or subsequences, including complementary sequences, can also be used in hybridization assays. A nucleotide encoding a Protein Isoform, or subsequences thereof comprising at least 8 nucleotides, preferably at least 12 nucleotides, and most preferably at least 15 nucleotides can be used as a hybridization probe. Hybridization assays can be used for detection, treatment, diagnosis, or monitoring of conditions, disorders, or disease states, associated with aberrant expression of genes encoding Protein Isoforms, or for differential diagnosis of subjects with signs or symptoms suggestive of neurological disorder. In particular, such a hybridization assay can be carried out by a method comprising contacting a subject's sample containing nucleic acid with a nucleic acid probe capable of hybridizing to a DNA or RNA that encodes a Protein Isoform, under conditions such that hybridization can occur, and detecting or measuring any resulting hybridization. Nucleotides can be used for therapy of subjects having a neurological disorder, as described below.

The invention also provides diagnostic kits, comprising an anti-Protein Isoform antibody. In addition, such a kit may optionally comprise one or more of the following: (1) instructions for using the anti-Protein Isoform antibody for diagnosis, prognosis, therapeutic monitoring or any suitable combination of these applications; (2) a labeled binding partner to the antibody; (3) a solid phase (such as a reagent strip) upon which the anti-Protein Isoform antibody is immobilized; and (4) a label or insert indicating regulatory approval for diagnostic, prognostic or therapeutic use or any suitable combination thereof. If no labeled binding partner to the antibody is provided, the anti-Protein Isoform antibody itself can be labeled with a detectable marker, e.g., a chemiluminescent, enzymatic, fluorescent, or radioactive moiety.

The invention also provides a kit comprising a nucleic acid probe capable of hybridizing to RNA encoding a Protein Isoform. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a Protein Isoform, such as by polymerase chain reaction (see, e.g., Innis et al., 1990, PCR Protocols, Academic Press, Inc., San Diego, Calif.), ligase chain reaction (see EP 320,308) use of Qβ, replicase, cyclic probe reaction, or other methods known in the art.

Kits are also provided which allow for the detection of a plurality of Protein Isoforms or a plurality of nucleic acids each encoding a Protein Isoform. A kit can optionally further comprise predetermined amounts of an isolated Protein Isoform protein or a nucleic acid encoding a Protein Isoform, e.g., for use as a standard or control.

### 5.3 Use in Clinical Studies

The diagnostic methods and compositions of the present invention can assist in monitoring a clinical study, e.g. to evaluate therapies for neurological disorder. In one embodiment, candidate molecules are tested for their ability to restore Protein Isoform levels in a subject having neurological disorder to levels found in subjects free from a neurological disorder or, in a treated subject (e.g. after treatment for depression with: mood stabilizers—lithium, divalproex, carbamazepine, lamotrigine; antidepressants—tricyclic antidepressants (e.g. Desipramine, chlorimipramine, nortriptyline), selective serotonin reuptake inhibitors (SSRIs including fluoxetine (Prozac), sertraline (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: Clozapine, Olanzapine, Risperidone. After treatment for Schizophrenia with Haloperidol, Pirenzepine, Perazine, Risperdal, Famotidine, Zyprexa, Clozaril, Mesoridazine, Quetiapine, atypical anti-psychotic medications of Risperidone, Olanzapine and Clozapine and any other Dibenzothiazepines. After treatment for Vascular dementia with: antiplatelet agents such as aspirin, Buflomedil (Cucinotta et al. J Int Med Res (1992) 20:136-49), neuroprotective agents such as Propentofylline (Rother et al. Ann N Y Acad Sci (1996) 777:404-9, Mielke et al. Alzheimer Dis Assoc Disord (1998) 12 Suppl 2:S29-35, Rother et al. Dement Geriatr Cogn Disord (1998) 9 Suppl 1:36-43), acetylcholinesterase inhibitors such as rivastigmine, galantamine (Kumar et al. Neurology (1999) 52 Suppl 2:A395) and other cytoprotective agents currently under clinical evaluation such as the calcium antagonists Nimodipine and Nicadipine, NMDA antagonists such as Selfotel, Dextrorphan, Cerestat, Eliprodil, Lamotrigine, GABA agonists, Kappa-selective opiod antagonists, Lubeluzole, Free radical scavengers, anti-ICAM antibodies and GM-1 ganglioside, Abbokinase®, Activase®, Aggrenox®, Anti-ICAM-1 antibody, Anti-beta-2-integrin antibody, Arvin®, Atacand®, CerAxon®, Cerebyx®, Ceresine®, Cerestat®, Cervene®, Coumadin®, Fiblast®, Fraxiparine®, Freedox®, Innohep®, Kabikinase®, Klerval®, LeukArrest®, Lipitor®, Lovenox®, Neurogard®, Nimotop®, Orgaran®, Persantine®, Plavix®, Prolyse®, Prosynap®, ReoPro®, Selfotel®, Sibelium®, Streptase®, Streptokinase, Sygen®, Ticlid®, Trental®, Viprinex®, Warfarin, Zanaflex®, Zendra®. After treatment for Alzheimer's disease with: an acetylcholinesterase inhibitor. After treatment for relapsing-remitting MS with: Interferon β-1b (Betaseron®, Betaferon®), Interferon β-1 a (Avonex®, Rebif®), Glatiramer acetate (Copaxone®), intravenous immunoglobulin and for acute relapse therapies with corticosteroids (Noseworthy (1999) Nature 399:suppl. A40-A47)) to preserve Protein Isoform levels at or near non-neurological disorder values. The levels of one or more Protein Isoforms can be assayed

In another embodiment, the methods and compositions of the present invention are used to screen individuals for entry into a clinical study to identify individuals having a neurological disorder; individuals already having a neurological disorder can then be excluded from the study or can be placed in a separate cohort for treatment or analysis. If desired, the candidates can concurrently be screened to identify individuals with specific conditions; procedures for these screens are well known in the art.

In a preferred embodiment, the methods and compositions of the present invention are used in the context of neurological disease and treatments affecting the glutamate pathway. Such treatments include, but are not limited to: the glutamate antagonist lamotrigine (Lamictal) for schizophrenia and depression, and Ebixa (Lundbeck), Memantamine (Merz) and Neramexane (Forest Laboratories) for Alzheimer's Disease.

### 5.4 Purification of Protein Isoforms

In particular aspects, the invention provides isolated mammalian Protein Isoforms, preferably human Protein Isoforms, and fragments thereof, which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) Protein Isoform, e.g., binding to a Protein Isoform substrate or Protein Isoform binding partner, antigenicity (binding to an anti-Protein Isoform antibody), immunogenicity, enzymatic activity and the like.

In specific embodiments, the invention provides fragments of a Protein Isoform comprising at least 5 amino acids, at least 10 amino acids, at least 50 amino acids, or at least 75 amino acids. Fragments lacking some or all of the regions of a Protein Isoform are also provided, as are proteins (e.g., fusion proteins) comprising such fragments. Nucleic acids encoding the foregoing are provided.

Once a recombinant nucleic acid that encodes the Protein Isoform, a portion of the Protein Isoform, or a precursor of the Protein Isoform is identified, the gene product can be analyzed. This can be achieved by assays based on the physical or functional properties of the given product, including, for example, radioactive labeling of the product followed by analysis by gel electrophoresis, immunoassay, etc.

The Protein Isoforms identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the Protein Isoform is identified, the entire amino acid sequence of the Protein Isoform can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native Protein Isoforms can be purified from natural sources, by standard methods such as those described above (e.g., immunoaffinity purification).

In a preferred embodiment, Protein Isoforms are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated Protein Isoform that can be recovered from the gel. When used in this way for preparative-scale runs, the Preferred Technology typically provides up to 100 ng, and can provide up to 1000 ng, of an isolated Protein Isoform in a single run. Those of skill in the art will appreciate that a zoom gel can be used in any separation strategy which employs gel isoelectric focusing.

The invention thus provides an isolated Protein Isoform, an isolated Protein Isoform-related polypeptide, and an isolated derivative or fragment of a Protein Isoform or a Protein Isoform-related polypeptide; any of the foregoing can be produced by recombinant DNA techniques or by chemical synthetic methods.

### 5.5 Isolation of DNA Encoding a Protein Isoform

Particular embodiments for the cloning of a gene encoding a Protein Isoform, are presented below by way of example and not of limitation.

The nucleotide sequences of the present invention, including DNA and RNA, and comprising a sequence encoding a Protein Isoform or a fragment thereof, or a Protein Isoform-related polypeptide, may be synthesized using methods known in the art, such as using conventional chemical approaches or polymerase chain reaction (PCR) amplification. The nucleotide sequences of the present invention also permit the identification and cloning of the gene encoding a Protein Isoform homolog or Protein Isoform ortholog including, for example, by screening cDNA libraries, genomic libraries or expression libraries.

For example, to clone a gene encoding a Protein Isoform by PCR techniques, anchored degenerate oligonucleotides (or a set of most likely oligonucleotides) can be designed for all Protein Isoform peptide fragments identified as part of the same protein. PCR reactions under a variety of conditions can be performed with relevant cDNA and genomic DNAs (e.g., from brain tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end (Arnold C, 1991, PCR Methods Appl. 1(1):39-42; Dyer ICD, Biotechniques, 1995, 19(4):550-2). Vectorette PCR may be performed with probes that are, for example, anchored degenerate oligonucleotides (or most likely oligonucleotides) coding for Protein Isoform peptide fragments, using as a template a genomic library or cDNA library pools.

Anchored degenerate oligonucleotides (and most likely oligonucleotides) can be designed for all Protein Isoform peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding a Protein Isoform or Protein Isoform fragment of the present invention are useful, for example, for their ability to hybridize selectively with complementary stretches of genes encoding other proteins. Depending on the application, a variety of hybridization conditions may be employed to obtain nucleotide sequences at least about 30%, 35%, 40%, 45%, 50%, 55%, 60%, 65%, 70%, 75%, 80%, 85%, 90%, 95% or 99% identical, or 100% identical, to the sequence of a nucleotide encoding a Protein Isoform.

For a high degree of selectivity, relatively stringent conditions are used to form the duplexes, such as low salt or high temperature conditions. As used herein, “highly stringent conditions” means hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding a Protein Isoform, 37° C. for 90 to 95% identity and 32° C. for 70 to 90% identity.

In the preparation of genomic libraries, DNA fragments are generated, some of which will encode parts or the whole of a Protein Isoform. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, New York; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labeled degenerate oligonucleotide probes corresponding to the amino acid sequence of any peptide of the Protein Isoform using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

For any Protein Isoform, degenerate probes, or probes taken from the sequences described above by accession number may be used for screening. In the case of degenerate probes, they can be constructed from the partial amino sequence information obtained from tandem mass spectra of tryptic digest peptides of the Protein Isoform. To screen such a gene, any probe may be used that is complementary to the gene or its complement; preferably the probe is 10 nucleotides or longer, more preferably 15 nucleotides or longer.

When a library is screened, clones with insert DNA encoding the Protein Isoform of interest or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridization.

In yet another aspect of the invention, clones containing nucleotide sequences encoding the entire Protein Isoform, a fragment of a Protein Isoform, a Protein Isoform-related polypeptide, or a fragment of a Protein Isoform-related polypeptide or any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed Protein Isoform or Protein Isoform-related polypeptides. In one embodiment, the various anti-Protein Isoform antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes a Protein Isoform, a fragment of a Protein Isoform, a Protein Isoform-related polypeptide, or a fragment of a Protein Isoform-related polypeptide can be detected using DYNA Beads according to Olsvick et al., 29th ICAAC, Houston, Tex. 1989, incorporated herein by reference. Anti-Protein Isoform antibodies are crosslinked to tosylated DYNA Beads M280, and these antibody-containing beads are then contacted with colonies or plaques expressing recombinant polypeptides. Colonies or plaques expressing a Protein Isoform or Protein Isoform-related polypeptide are identified as any of those that bind the beads.

Alternatively, the anti-Protein Isoform antibodies can be nonspecifically immobilized to a suitable support, such as silica or Celite7 resin. This material is then used to adsorb to bacterial colonies expressing the Protein Isoform protein or Protein Isoform-related polypeptide as described herein.

In another aspect, PCR amplification may be used to isolate from genomic DNA a substantially pure DNA (i.e., a DNA substantially free of contaminating nucleic acids) encoding the entire Protein Isoform or a part thereof. Preferably such a DNA is at least 95% pure, more preferably at least 99% pure. Oligonucleotide sequences, degenerate or otherwise, that correspond to peptide sequences of Protein Isoforms disclosed herein can be used as primers.

PCR can be carried out, e.g., by use of a Perkin-Elmer Cetus thermal cycler and Taq polymerase (Gene Amp? or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding a Protein Isoform, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding a Protein Isoform can also be identified by mRNA selection by nucleic acid hybridization followed by in vitro translation. In this procedure, fragments are used to isolate complementary mRNAs by hybridization. Such DNA fragments may represent available, purified DNA encoding a Protein Isoform of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize a Protein Isoform. A radiolabelled cDNA encoding a Protein Isoform can be synthesized using the selected mRNA (from the adsorbed polysomes) as a template. The radiolabelled mRNA or cDNA may then be used as a probe to identify the DNA fragments encoding a Protein Isoform from among other genomic DNA fragments.

Alternatives to isolating genomic DNA encoding a Protein Isoform include, but are not limited to, chemically synthesizing the gene sequence itself from a known sequence or making cDNA to the mRNA which encodes the Protein Isoform. For example, RNA for cDNA cloning of the gene encoding a Protein Isoform can be isolated from cells that express the Protein Isoform. Those skilled in the art will understand from the present description that other methods may be used and are within the scope of the invention.

Any suitable eukaryotic cell can serve as the nucleic acid source for the molecular cloning of the gene encoding a Protein Isoform. The nucleic acid sequences encoding the Protein Isoform can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the vector system chosen should be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding a Protein Isoform may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the Protein Isoform, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native Protein Isoform, nucleotide sequences encoding amino acid sequences with functionally equivalent amino acids, nucleotide sequences encoding Protein Isoforms, fragments of Protein Isoforms, Protein Isoform-related polypeptides, or fragments of Protein Isoform-related polypeptides.

In a specific embodiment, an isolated nucleic acid molecule encoding a Protein Isoform-related polypeptide can be created by introducing one or more nucleotide substitutions, additions or deletions into the nucleotide sequence of a Protein Isoform such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of, the coding sequence, such as by saturation mutagenesis, and the resultant mutants can be screened for biological activity to identify mutants that retain activity. Following mutagenesis, the encoded protein can be expressed and the activity of the protein can be determined.

### 5.6 Expression of DNA Encoding Protein Isoforms

The nucleotide sequence coding for a Protein Isoform, a Protein Isoform analog, a Protein Isoform-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the Protein Isoform or its flanking regions, or the native gene encoding the Protein Isoform-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a, human gene (or a nucleotide sequence encoding a functionally active portion of a human Protein Isoform) is expressed. In yet another embodiment, a fragment of a Protein Isoform comprising a domain of the Protein Isoform is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding a Protein Isoform or fragment thereof may be regulated by a second nucleic acid sequence so that the Protein Isoform or fragment is expressed in a host transformed with the recombinant DNA molecule. For example, expression of a Protein Isoform may be controlled by any promoter or enhancer element known in the art. Promoters which may be used to control the expression of the gene encoding a Protein Isoform or a Protein Isoform-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a Protein Isoform-encoding nucleic acid, one or more origins of replication, and, optionally, one or more selectable markers (e.g., an antibiotic resistance gene).

In a specific embodiment, an expression construct is made by subcloning a Protein Isoform or a Protein Isoform-related polypeptide coding sequence into the EcoRI restriction site of each of the three pGEX vectors (Glutathione S-Transferase expression vectors; Smith and Johnson, 1988, Gene 7:31-40). This allows for the expression of the Protein Isoform product or Protein Isoform-related polypeptide from the subclone in the correct reading frame.

In mammalian host cells, a number of viral-based expression systems may be utilized. In cases where an adenovirus is used as an expression vector, the Protein Isoform coding sequence or Protein Isoform-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts, (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a Protein Isoform or a Protein Isoform-related polypeptide can be identified, for example, by three general approaches: (a) nucleic acid hybridization, (b) presence or absence of “marker” gene functions, and (c) expression of inserted sequences. In the first approach, the presence of a gene encoding a Protein Isoform inserted in an expression vector can be detected by nucleic acid hybridization using probes comprising sequences that are homologous to an inserted gene encoding a Protein Isoform. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a Protein Isoform in the vector. For example, if the gene encoding the Protein Isoform is inserted within the marker gene sequence of the vector, recombinants containing the gene encoding the Protein Isoform insert can be identified by the absence of the marker gene function. In the third approach, recombinant expression vectors can be identified by assaying the gene product (i.e., Protein Isoform) expressed by the recombinant. Such assays can be based, for example, on the physical or functional properties of the Protein Isoform in in vitro assay systems, e.g., binding with anti-Protein Isoform antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered Protein Isoform or Protein Isoform-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK 293, 3T3, WI38, and in particular neuronal cell lines such as, for example, SK-N-AS, SK-N-FI, SK-N-DZ human neuroblastomas (Sugimoto T et al. 1984 J. Natl. Cancer Inst. 73, 51-57), SK-N-SH human neuroblastoma (Biochim. Biophys. Acta 1982 704, 450-460), Daoy human cerebellar medulloblastoma (He et al. 1992 Cancer Res. 52, 1144-1148) DBTRG-05MG glioblastoma cells (Kruse et al., 1992 In Vitro Cell. Dev. Biol. 28A, 609-614), IMR-32 human neuroblastoma (Cancer Res. 1970 30, 2110-2118), 1321N1 human astrocytoma (Proc Natl Acad Sci USA 1977 74, 4816), MOG-G-CCM human astrocytoma (Br J Cancer 1984 49, 269), U87MG human glioblastoma-astrocytoma (Acta Pathol Microbiol Scand 1968; 74:465-486), A172 human glioblastoma (Olopade et al., 1992 Cancer Res. 52: 2523-2529), C6 rat glioma cells (Benda et al. 1968 Science 161, 370-371), Neuro-2a mouse neuroblastoma (Proc. Natl. Acad. Sci. USA 1970 65, 129-136), N1341A3 mouse neuroblastoma (Proc. Natl. Acad. Sci. USA 1962 48, 1184-1190), SCP sheep choroid plexus (Bolin et al. 1994 J. Virol. Methods 48, 211-221), G355-5, PG-4 Cat normal astrocyte (Haapala et al. 1985 J Virol. 53, 827-833), Mpf ferret brain (Trowbridge et al. 1982 In Vitro 18 952-960), and normal cell lines such as, for example, CTX TNA2 rat normal cortex brain (Radany et al. 1992 Proc. Natl. Acad. Sci. USA 89, 6467-6471). Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other embodiments, the Protein Isoform, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins, see, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding a Protein Isoform, a fragment of a Protein Isoform, a Protein Isoform-related polypeptide, or a fragment of a Protein Isoform-related polypeptide can be fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

A Protein Isoform fusion protein can be made by ligating the appropriate nucleic acid sequences encoding the desired amino acid sequences to each other by methods known in the art, in the proper coding frame, and expressing the chimeric product by methods commonly known in the art. Alternatively, a Protein Isoform fusion protein may be made by protein synthetic techniques, e.g., by use of a peptide synthesizer.

Both cDNA and genomic sequences can be cloned and expressed.

### 5.7 Domain Structure of Protein Isoforms

Domains of some of the Protein Isoforms provided by the present invention are known in the art and have been described in the scientific literature. Moreover, domains of a Protein Isoform can be identified using techniques known to those of skill in the art. For example, one or more domains of a Protein Isoform can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occurring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, those skilled in the art can identify domains of a Protein Isoform having enzymatic or binding activity, and further can identify nucleotide sequences encoding such domains. These nucleotide sequences can then be used for recombinant expression of a Protein Isoform fragment that retains the enzymatic or binding activity of the Protein Isoform.

Based on the present description, those skilled in the art can identify domains of a Protein Isoform having enzymatic or binding activity, and further can identify nucleotide sequences encoding such domains. These nucleotide sequences can then be used for recombinant expression of Protein Isoform fragments that retain the enzymatic or binding activity of the Protein Isoform.

In one embodiment, a Protein Isoform has an amino acid sequence sufficiently similar to an identified domain of a known polypeptide. As used herein, the tenor “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A Protein Isoform domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay. In a preferred embodiment, the function of a domain of a Protein Isoform is determined using an assay described in one or more of the references identified in Table III, infra.

### 5.8 Production of Affinity Reagents to Protein isoforms

According to those in the art, there are three main types of affinity reagent—monoclonal antibodies, phage display antibodies and small molecules such as Affibodies or Domain Antibodies (dAbs). In general in applications according to the present invention where the use of antibodies is stated, other affinity reagents (eg Affibodies or domain antibodies) may be employed.

### 5.8.1 Production of Antibodies to Protein Isoforms

According to the invention a Protein Isoform, Protein Isoform analog, Protein Isoform-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding Protein Isoforms may be prepared. For example, antibodies that recognize these Protein Isoforms and/or their isoforms include the antibodies recognizing Protein Isoforms listed in Table I above. Certain antibodies are already known and can be purchased from commercial sources as shown in Table II. In another embodiment, methods known to those skilled in the art are used to produce antibodies that recognize a Protein Isoform, a Protein Isoform analog, a Protein Isoform-related polypeptide, or a derivative or fragment of any of the foregoing.

In one embodiment of the invention, antibodies to a specific domain of a Protein Isoform are produced. In a specific embodiment, hydrophilic fragments of a Protein Isoform are used as immunogens for antibody production.

In the production of antibodies, screening for the desired antibody can be accomplished by techniques known in the art, e.g. ELISA (enzyme-linked immunosorbent assay). For example, to select antibodies that recognize a specific domain of a Protein Isoform, one may assay generated hybridomas for a product that binds to a Protein Isoform fragment containing such domain. For selection of an antibody that specifically binds a first Protein Isoform homolog but which does not specifically bind to (or binds less avidly to) a second Protein Isoform homolog, one can select on the basis of positive binding to the first Protein Isoform homolog and a lack of binding to (or reduced binding to) the second Protein Isoform homolog. Similarly, for selection of an antibody that specifically binds a Protein Isoform but which does not specifically bind to (or binds less avidly to) a different isoform of the same protein (such as a different glycoform having the same core peptide as the Protein Isoform), one can select on the basis of positive binding to the Protein Isoform and a lack of binding to (or reduced binding to) the different isoform (e.g., a different glycoform). Thus, the present invention provides an antibody (particularly a monoclonal antibody) that binds with greater affinity (particularly at least 2-fold, more particularly at least 5-fold still more particularly at least 10-fold greater affinity) to a Protein Isoform than to a different isoform or isoforms (e.g., glycoforms) of the Protein Isoform.

Polyclonal antibodies which may be used in the methods of the invention are heterogeneous populations of antibody molecules derived from the sera of immunized animals, unfractionated immune serum can also be used. Various procedures known in the art may be used for the production of polyclonal antibodies to a Protein Isoform, a fragment of a Protein Isoform, a Protein Isoform-related polypeptide, or a fragment of a Protein Isoform-related polypeptide. In a particular embodiment, rabbit polyclonal antibodies to an epitope of a Protein Isoform or a Protein Isoform-related polypeptide can be obtained, for example, for the production of polyclonal or monoclonal antibodies, various host animals can be immunized by injection with the native or a synthetic (e.g., recombinant) version of a Protein Isoform, a fragment of a Protein Isoform, a Protein Isoform-related polypeptide, or a fragment of a Protein Isoform-related polypeptide, including but not limited to rabbits, mice, rats, etc. Isolated Protein Isoforms suitable for such immunization may be obtained by the use of discovery techniques, such as the preferred technology described herein. If the Protein Isoform is purified by gel electrophoresis, the Protein Isoform can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum, additional adjuvants are also well known in the art.

For preparation of monoclonal antibodies (mAbs) directed toward a Protein Isoform, a fragment of a Protein Isoform, a Protein Isoform-related polypeptide, or a fragment of a Protein Isoform-related polypeptide, any technique which provides for the production of antibody molecules by continuous cell lines in culture may be used, for example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA, 84:3439-3443; Liu et al., 1987, J. Immunol. 139:3521-3526; Sun et al., 1987, Proc. Natl. Acad. Sci. USA, 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science, 239:1534; and Beidler et al., 1988, J. Immunol. 141:4053-4060.

Completely human antibodies are particularly desirable for therapeutic treatment of human subjects, such antibodies can be produced using transgenic mice which are incapable of expressing endogenous immunoglobulin heavy and light chains genes, but which can express human heavy and light chain genes. The transgenic mice are immunized in the normal fashion with selected antigens, e.g., all or a portion of a Protein Isoform of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection”, in this approach a selected non-human monoclonal antibody, e.g., a mouse antibody, is used to guide the selection of a completely human antibody recognizing the same epitope, (Jespers et al. (1994) Biotechnology 12:899-903).

The antibodies of the present invention can also be generated using various phage display methods known in the art. In phage display methods, functional antibody domains are displayed on the surface of phage particles that carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al, Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of suitable techniques which can be used to produce single-chain Fvs and antibodies against Protein Isoforms of the present invention include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology,1986, 121:210.

The invention provides functionally active fragments, derivatives or analogs of the anti-Protein Isoform immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any suitable binding assay known in the art.

The present invention provides antibody fragments such as, but not limited to, F(ab′)2 fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immununospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical or unnatural amino acids.

The foregoing antibodies can be used in methods known in the art relating to the localization and activity of the Protein Isoforms of the invention, e.g., for imaging these proteins, measuring levels thereof in appropriate physiological samples, in diagnostic methods, etc.

### 5.8.2 Production of Affibodies to Protein Isoforms

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labelled Affibodies may also be useful in imaging appkications for determining abundance of Isoforms.

### 5.8.3 Domain Antibodies

Domain Antibodies (dAbs) are the smallest functional binding units of antibodies, corresponding to the variable regions of either the heavy (V_{H}) or light (V_{L}) chains of human antibodies Domain Antibodies have a molecular weight of approximately 13 kDa. Domantis has developed a series of large and highly functional libraries of fully human V_{H} and V_{L} dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609. each of which is herein incorporated by reference in its entirety.

### Selection of Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹/W, about 10⁹ M⁻¹ to about 10¹⁰ M⁻¹, or about 10¹⁰ M⁻¹ to about 10¹¹ M⁻¹.

Affinity is calculated as K_{d}=k_{off}kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microliter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

For therapeutic applications, antibodies (particularly monoclonal antibodies) may suitably be human or humanized animal (eg mouse) antibodies. Animal antibodies may be raised in animals using the human protein (eg a CRCMP) as immunogen. Humanisation typically involves grafting CDRs identified thereby into human framework regions. Normally some subsequent retromutation to optimize the conformation of chains is required. Such processes are known to persons skilled in the art.

### 5.9 Expression of Affinity Reagents

### 5.9.1 Expression of Monoclonal Antibodies

The antibodies of the invention can be produced by any suitable method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen based on the present description which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+ nitriloacetic acid-agarose columns and histidine-tagged proteins are selectively eluted with imidazole-containing buffers.

### 5.9.1 Expression of Affibodies

### Selection and Production of Affibody Variants

The construction of affibodies has been described elsewhere (Ronnmark J, Gronlund H, Uhle' n, M., Nygren P. A°, Human immunoglobulin A (IgA)-specific ligands from combinatorial engineering of protein A, 2002, Eur. J. Biochem. 269, 2647-2655), including the construction of affibody phage display libraries (Nord, K., Nilsson, J., Nilsson, B., Uhle' n, M. & Nygren, P. A°, A combinatorial library of an a-helical bacterial receptor domain, 1995, Protein Eng. 8, 601-608. Nord, K., Gunneriusson, E., Ringdahl, J., Sta° hl, S., Uhle' n, M. & Nygren, P. A°, Binding proteins selected from combinatorial libraries of an a-helical bacterial receptor domain, 1997, Nat. Biotechnol. 15, 772-777.)

The biosensor analyses to investigate the optimal affibody variants using biosensor binding studies has also been described elsewhere (Ronnmark J, Gronlund H, Uhle' n, M., Nygren P. A°, Human immunoglobulin A (IgA)-specific ligands from combinatorial engineering of protein A, 2002, Eur. J. Biochem. 269, 2647-2655),

### 5.10 Conjugated Affinity Reagents

In a preferred embodiment, anti-Protein Isoform affinity reagents such as antibodies or fragments thereof are conjugated to a diagnostic or a therapeutic moiety. The antibodies can be used, for example, for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc. ⁶⁸Ga may also be employed

Anti-Protein Isoform antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan it Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). These references are incorporated herein in their entirety.

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

### 5.11 Diagnosis of Neurological disorder

In accordance with the present invention, suitable test samples, e.g., of CSF, obtained from a subject suspected of having or known to have neurological disorder can be used for diagnosis. In one embodiment, an altered abundance of one or more Protein Isoforms in a test sample relative to a control sample (from a subject or subjects free from neurological disorder) or a previously determined reference range indicates the presence of neurological disorder; Protein Isoforms suitable for this purpose are identified in Table I as described in detail above. In another embodiment, the relative abundance of one or more Protein Isoforms in a test sample compared to a control sample or a previously determined reference range indicates a subtype of neurological disorder (e.g., a familial or sporadic variant of a neurological disorder). In yet another embodiment, the relative abundance of one or more Protein Isoforms (or any combination of them) in a test sample relative to a control sample or a previously determined reference range indicates the degree or severity of a neurological disorder. In any of the aforesaid methods, detection of one or more Protein Isoforms described herein may optionally be combined with detection of one or more additional biomarkers for neurological disorder including, but not limited to but not limited to apoplipoprotein E (ApoE), amyloid β-peptides (Aβ), tau and neural thread protein (NTP), oligoclonal immunoglobulin bands in CSF revealed by isoelectric focusing (Reiber H et al. (1998) Mult Scler 3: 111-7). Any suitable method in the art can be employed to measure the level of Protein Isoforms, including but not limited to the Preferred Technology described herein, kinase assays, immunoassays to detect and/or visualize the Protein Isoforms (e.g., Western blot, immunoprecipitation followed by sodium dodecyl sulfate polyacrylamide gel electrophoresis, immunocytochemistry, etc.). In cases where a Protein Isoform has a known function, an assay for that function may be used to measure Protein Isoform expression. In a further embodiment, an altered abundance of mRNA encoding one or more Protein Isoforms identified in Table I (or any combination of them) in a test sample relative to a control sample or a previously determined reference range indicates the presence of neurological disorder. Any suitable hybridization assay can be used to detect Protein Isoform expression by detecting and/or visualizing mRNA encoding the Protein Isoform (e.g., Northern assays, dot blots, in situ hybridization, etc.).

In another embodiment of the invention, labeled antibodies, derivatives and analogs thereof, which specifically bind to a Protein Isoform can be used for diagnostic purposes, e.g., to detect, diagnose, or monitor neurological disorder. Preferably, neurological disorder is detected in an animal, more preferably in a mammal and most preferably in a human.

### 5.12 Screening Assays

The invention provides methods for identifying agents (e.g., chemical compounds, proteins, or peptides) that bind to a Protein Isoform or have a stimulatory or inhibitory effect on the expression or activity of a Protein Isoform. The invention also provides methods of identifying agents, candidate compounds or test compounds that bind to a Protein Isoform-related polypeptide or a Protein Isoform fusion protein or have a stimulatory or inhibitory effect on the expression or activity of a Protein Isoform-related polypeptide or a Protein Isoform fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des., 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mot Biol. 222:301-310), each of which is incorporated herein in its entirety by reference.

In one embodiment, agents that interact with (i.e., bind to) a Protein Isoform, a Protein Isoform fragment (e.g. a functionally active fragment), a Protein Isoform-related polypeptide, a fragment of a Protein Isoform-related polypeptide, or a Protein Isoform fusion protein are identified in a cell-based assay system. In accordance with this embodiment, cells expressing a Protein Isoform, a fragment of a Protein Isoform, a Protein Isoform-related polypeptide, a fragment of a Protein Isoform-related polypeptide, or a Protein Isoform fusion protein are contacted with a candidate compound or a control compound and the ability of the candidate compound to interact with the Protein Isoform is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the Protein Isoform, fragment of the Protein Isoform, Protein Isoform-related polypeptide, a fragment of the Protein Isoform-related polypeptide, or a Protein Isoform fusion protein endogenously or be genetically engineered to express the Protein Isoform, fragment of the Protein Isoform, Protein Isoform-related polypeptide, a fragment of the Protein Isoform-related polypeptide, or a Protein Isoform fusion protein. In some embodiments, the Protein Isoform, fragment of the Protein Isoform, Protein Isoform-related polypeptide, a fragment of the Protein Isoform-related polypeptide, or a Protein Isoform fusion protein or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a Protein Isoform and a candidate compound. The ability of the candidate compound to interact directly or indirectly with a Protein Isoform, a fragment of a Protein Isoform, a Protein Isoform-related polypeptide, a fragment of a Protein Isoform-related polypeptide, or a Protein Isoform fusion protein can be determined by methods known to those of skill in the art. For example, the interaction between a candidate compound and a Protein Isoform, a fragment of a Protein Isoform, a Protein Isoform-related polypeptide, a fragment of a Protein Isoform-related polypeptide, or a Protein Isoform fusion protein can be determined by flow cytometry, a scintillation assay, immunoprecipitation or western blot analysis.

In another embodiment, agents that interact with (i.e., bind to) a Protein Isoform, a Protein Isoform fragment (e.g., a functionally active fragment) a Protein Isoform-related polypeptide, a fragment of a Protein Isoform-related polypeptide, or a Protein Isoform fusion protein are identified in a cell-free assay system. In accordance with this embodiment, a native or recombinant Protein Isoform or fragment thereof, or a native or recombinant Protein Isoform-related polypeptide or fragment thereof, or a Protein Isoform-fusion protein or fragment thereof, is contacted with a candidate compound or a control compound and the ability of the candidate compound to interact with the Protein Isoform or Protein Isoform-related polypeptide, or Protein Isoform fusion protein is determined. If desired, this assay may be used to screen a plurality (e.g. a library) of candidate compounds. Preferably, the Protein Isoform, Protein Isoform fragment, Protein Isoform-related polypeptide, a fragment of a Protein Isoform-related polypeptide, or a Protein Isoform-fusion protein is first immobilized, by, for example, contacting the Protein Isoform, Protein Isoform fragment, Protein Isoform-related polypeptide, a fragment of a Protein Isoform-related polypeptide, or a Protein Isoform fusion protein with an immobilized antibody which specifically recognizes and binds it, or by contacting a purified preparation of the Protein Isoform, Protein Isoform fragment, Protein Isoform-related polypeptide, fragment of a Protein Isoform-related polypeptide, or a Protein Isoform fusion protein with a surface designed to bind proteins. The Protein Isoform, Protein Isoform fragment, Protein Isoform-related polypeptide, a fragment of a Protein Isoform-related polypeptide, or a Protein Isoform fusion protein may be partially or completely purified (e.g., partially or completely free of other polypeptides) or part of a cell lysate. Further, the Protein Isoform, Protein Isoform fragment, Protein Isoform-related polypeptide, a fragment of a Protein Isoform-related polypeptide may be a fusion protein comprising the Protein Isoform or a biologically active portion thereof, or Protein Isoform-related polypeptide and a domain such as glutathionine-S-transferase. Alternatively, the Protein Isoform, Protein Isoform fragment, Protein Isoform-related polypeptide, fragment of a Protein Isoform-related polypeptide or Protein Isoform fusion protein can be biotinylated using techniques well known to those of skill in the art (e.g., biotinylation kit, Pierce Chemicals; Rockford, Ill.). The ability of the candidate compound to interact with a Protein Isoform, Protein Isoform fragment, Protein Isoform-related polypeptide, a fragment of a Protein Isoform-related polypeptide, or a Protein Isoform fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a Protein Isoform or is responsible for the post-translational modification of a Protein Isoform. In a primary screen, a plurality (e.g., a library) of compounds are contacted with cells that naturally or recombinantly express: (i) a Protein Isoform, an isoform of a Protein Isoform, a Protein Isoform homolog a Protein Isoform-related polypeptide, a Protein Isoform fusion protein, or a biologically active fragment of any of the foregoing; and (ii) a protein that is responsible for processing of the Protein Isoform, Protein Isoform isoform, Protein Isoform homolog, Protein Isoform-related polypeptide, Protein Isoform fusion protein, or fragment in order to identify compounds that modulate the production, degradation, or post-translational modification of the Protein Isoform, Protein Isoform isoform, Protein Isoform homolog, Protein Isoform-related polypeptide, Protein Isoform fusion protein or fragment. If desired, compounds identified in the primary screen can then be assayed in a secondary screen against cells naturally or recombinantly expressing the specific Protein Isoforms of interest. The ability of the candidate compound to modulate the production, degradation or post-translational modification of a Protein Isoform, isoform, homolog, Protein Isoform-related polypeptide, or Protein Isoform fusion protein can be determined by methods known to those of skill in the art, including without limitation, flow cytometry, a scintillation assay, immunoprecipitation and western blot analysis.

In another embodiment, agents that competitively interact with (i.e., bind to) a Protein Isoform, Protein Isoform fragment, Protein Isoform-related polypeptide, a fragment of a Protein Isoform-related polypeptide, or a Protein Isoform fusion protein are identified in a competitive binding assay. In accordance with this embodiment, cells expressing a Protein Isoform, Protein Isoform fragment, Protein Isoform-related polypeptide, a fragment of a Protein Isoform-related polypeptide, or a Protein Isoform fusion protein are contacted with a candidate compound and a compound known to interact with the Protein Isoform, Protein Isoform fragment, Protein Isoform-related polypeptide, a fragment of a Protein Isoform-related polypeptide or a Protein Isoform fusion protein; the ability of the candidate compound to competitively interact with the Protein Isoform, Protein Isoform fragment, Protein Isoform-related polypeptide, fragment of a Protein Isoform-related polypeptide, or a Protein Isoform fusion protein is then determined. Alternatively, agents that competitively interact with (i.e., bind to) a Protein Isoform, Protein Isoform fragment, Protein Isoform-related polypeptide or fragment of a Protein Isoform-related polypeptide are identified in a cell-free assay system by contacting a Protein Isoform, Protein Isoform fragment, Protein Isoform-related polypeptide, fragment of a Protein Isoform-related polypeptide, or a Protein Isoform fusion protein with a candidate compound and a compound known to interact with the Protein Isoform, Protein Isoform-related polypeptide or Protein Isoform fusion protein. As stated above, the ability of the candidate compound to interact with a Protein Isoform, Protein Isoform fragment, Protein Isoform-related polypeptide, a fragment of a Protein Isoform-related polypeptide, or a Protein Isoform fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of a Protein Isoform, or a Protein Isoform-related polypeptide are identified by contacting cells (e.g., cells of prokaryotic origin or eukaryotic origin) expressing the Protein Isoform, or Protein Isoform-related polypeptide with a candidate compound or a control compound (e.g., phosphate buffered saline (PBS)) and determining the expression of the Protein Isoform, Protein Isoform-related polypeptide, or Protein Isoform fusion protein, mRNA encoding the Protein Isoform, or mRNA encoding the Protein Isoform-related polypeptide. The level of expression of a selected Protein Isoform, Protein Isoform-related polypeptide, mRNA encoding the Protein Isoform, or mRNA encoding the Protein Isoform-related polypeptide in the presence of the candidate compound is compared to the level of expression of the Protein Isoform, Protein Isoform-related polypeptide, mRNA encoding the Protein Isoform, or mRNA encoding the Protein Isoform-related polypeptide in the absence of the candidate compound (e.g., in the presence of a control compound). The candidate compound can then be identified as a modulator of the expression of the Protein Isoform, or a Protein Isoform-related polypeptide based on this comparison. For example, when expression of the Protein Isoform or mRNA is significantly greater in the presence of the candidate compound than in its absence, the candidate compound is identified as a stimulator of expression of the Protein Isoform or mRNA. Alternatively, when expression of the Protein Isoform or mRNA is significantly less in the presence of the candidate compound than in its absence, the candidate compound is identified as an inhibitor of the expression of the Protein Isoform or mRNA. The level of expression of a Protein Isoform or the mRNA that encodes it can be determined by methods known to those of skill in the art based on the present description. For example, mRNA expression can be assessed by Northern blot analysis or RT-PCR, and protein levels can be assessed by western blot analysis.

In another embodiment, agents that modulate the activity of a Protein Isoform, or a Protein Isoform-related polypeptide are identified by contacting a preparation containing the Protein Isoform or Protein Isoform-related polypeptide, or cells (e.g., prokaryotic or eukaryotic cells) expressing the Protein Isoform or Protein Isoform-related polypeptide with a test compound or a control compound and determining the ability of the test compound to modulate (e.g., stimulate or inhibit) the activity of the Protein Isoform or Protein Isoform-related polypeptide. The activity of a Protein Isoform or a Protein Isoform-related polypeptide can be assessed by detecting induction of a cellular signal transduction pathway of the Protein Isoform or Protein Isoform-related polypeptide (e.g., intracellular Ca2+, diacylglycerol, IP3, etc.), detecting catalytic or enzymatic activity of the target on a suitable substrate, detecting the induction of a reporter gene (e.g., a regulatory element that is responsive to a Protein Isoform or a Protein Isoform-related polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities (see, e.g., U.S. Pat. No. 5,401,639, which is incorporated in its entirety herein by reference). The candidate agent can then be identified as a modulator of the activity of a Protein Isoform or Protein Isoform-related polypeptide by comparing the effects of the candidate compound to the control compound. Suitable control compounds include phosphate buffered saline (PBS) and normal same (NS).

In another embodiment, agents that modulate (i.e., upregulate or downregulate) the expression, activity or both the expression and activity of a Protein Isoform or Protein Isoform-related polypeptide are identified in an animal model. Examples of suitable animals include, but are not limited to, mice, rats, rabbits, monkeys, guinea pigs, dogs and cats. Preferably, the animal used represent a model of a neurological disorder (e.g., for depression a number of animal models have had significant value in the search for new treatments and in the study of mechanisms. Most notably, the Porsolt forced swim test model of depression is frequently used in both these contexts (Kirby and Lucid, 1997; Rossetti et al., 1993). The two major clinical states observed in bipolar disorder (depression and mania) have also been successfully modeled (Cappeliez and Moore Prog Neuropsychopharmacol Biol Psychiatry 1990 14, 347-58). Psychostimulant treatment can produce a range of behaviors similar to that of mania including hyperactivity, heightened sensory awareness, and alertness, and for this reason has become a very useful model for mania which exhibits (to some extent) face, construct and predictive validity. Another model that has been utilized for the development of bipolar illness is behavioral sensitization. In this model, the repeated administration of many psychostimulant drugs leads to a gradual increase or sensitization of the drug-induced behavioral; this model also has considerable construct and face validity for mania (Koob et al. Pharmacol Biochem Behav 1997 57, 513-21)). In accordance with this embodiment, the test compound or a control compound is administered (e.g., orally, rectally or parenterally such as intraperitoneally or intravenously) to a suitable animal and the effect on the expression, activity or both expression and activity of the Protein Isoform is determined. For Schizophrenia, e.g., Phencyclidine treated rodents (Sams-Dodd Rev Neurosci (1999) 10, 59-90), an animal model of deficient sensorimotor gating (Swerdlow and Geyer Schizophr Bull (1998) 24:2 285-301), neonatal insult to the hippocampal region (Beauregard and Bachevalier Can J Psychiatry (1996) September 41:7 446-56), models based on neonatal excitotoxic hippocampal damage (Lillrank et al, Clin Neurosci (1995) 3:2 98-104), attention deficit models (Feldon et al, J Psychiatr Res 4, 345-66) and NMDA deficient rodent models (Mohn et al, Cell (1999) 98, 427-436). For Vascular Dementia e.g., animal models of cerebral beta-amyloid angiopathy (Walker, Brain Res. Rev. (1997) 25:70-84), animal models of vascular dementia with emphasis on stroke-prone spontaneously hypertensive rats (Saito et al., Clin. Exp. Pharmacol. Physiol. (1995) 22 Suppl 1:s257-259), unilateral middle cerebral artery (MCA) occlusion, multiple small embolization or transient four-vessel occlusion model rats producing acute single or multi-infarct dementia memory impairments (Naritomi, Alzheimer Dis Assoc Disord (1991) 5:103-11), stroke-prone spontaneously hypertensive rats (Togashi et al., Stroke (1996) 27:520-5; discussion 525-6), Icelandic-like mutation of Cystatin C in an animal model of cerebrovascular beta-amyloidosis (Wei, Stroke (1996) 27:2080-5), and finally common models of stroke as reviewed by Feuerstein and Wang (Feuerstein G Z, Wang X, Animal models of stroke, Mol Med Today 2000 March; 6(3):133-5). For Alzheimer's disease—e.g., animals that express human familial Alzheimer's disease (FAD) β-amyloid precursor (APP), animals that overexpress human wild-type APP, animals that overexpress β-amyloid 1-42 (βA), animals that express FAD presenillin-1 (PS-1). See, e.g., Higgins, L S, 1999, Molecular Medicine Today 5:274-276. For Multiple Sclerosis, e.g., experimental autoimmune encephalomyelitis (EAE) (Steinman (1999) Neuron, 24:511-514)). In accordance with this embodiment, the test compound or a control compound is administered (e.g., orally, rectally or parenterally such as intraperitoneally or intravenously) to a suitable animal and the effect on the expression, activity or both expression and activity of the Protein Isoform or Protein Isoform-related polypeptide is determined. Changes in the expression of a Protein Isoform or Protein Isoform-related polypeptide can be assessed by any suitable method described above, based on the present description.

In yet another embodiment, a Protein Isoform or Protein Isoform-related polypeptide is used as a “bait protein” in a two-hybrid assay or three hybrid assay to identify other proteins that bind to or interact with a Protein Isoform or Protein Isoform-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the Protein Isoforms of the invention as, for example, upstream or downstream elements of a signaling pathway involving the Protein Isoforms of the invention.

As those skilled in the art will appreciate, Table III enumerates scientific publications describing suitable assays for detecting or quantifying enzymatic or binding activity of a Protein Isoform, a Protein Isoform analog, a Protein Isoform-related polypeptide, or a fragment of any of the foregoing. Each such reference is hereby incorporated in its entirety. In a preferred embodiment, an assay referenced in Table III is used in the screens and assays described herein, for example, to screen for or to identify an agent that modulates the activity of (or that modulates both the expression and activity of) a Protein Isoform, Protein Isoform analog, or Protein Isoform-related polypeptide, a fragment of any of the foregoing or a Protein Isoform fusion protein.

TABLE III

| | |
|---|---|
| PFI-# | Assay reference |

Heparin and Gags binding:

| | |
|---|---|
| PIF-1 and | Alberdi, E., Hyde, C. C. & Becerra, S. P. |
| PIF-2 | (1998) Biochemistry 37, 10643-10652. |
| | Stratikos, E., Alberdi, E., Gettins, P. G. & |
| | Becerra, S. P. (1996) Protein Sci. 5, 2575-2582. |

Neurotrophic Activity:

| | |
|---|---|
| PIF-1 and | Alberdi, E., Aymerich, M. S., and Becerra, |
| PIF-3 | S. P. (1999) J. Biol. Chem. 274, 31605-31612 |
| | Aymerich, M. S., Alberdi, E. M., Martinez, A., |
| | and Becerra, S. P. (2001) Investig. |
| | Ophthalmol. Vis. Sci. 42, 3287-3293 |
| | Yabe, T., Wilson, D., and Schwartz, J. P. |
| | (2001) J. Biol. Chem. 276, 43313-43319 |

This invention further provides novel agents identified by the above-described screening assays and uses thereof for treatments as described herein.

### 5.13 Therapeutic Uses of Protein Isoforms

The invention provides for treatment or prevention of various diseases and disorders by administration of a therapeutic agent. Such agents include but are not limited to: Protein Isoforms, Protein Isoform analogs, Protein Isoform-related polypeptides and derivatives (including fragments) thereof; antibodies to the foregoing; nucleic acids encoding Protein Isoforms, Protein Isoform analogs, Protein Isoform-related polypeptides and fragments thereof; antisense nucleic acids to a gene encoding a Protein Isoform or Protein Isoform-related polypeptide; and modulator (e.g., agonists and antagonists) of a gene encoding a Protein Isoform or Protein Isoform-related polypeptide. An important feature of the present invention is the identification of genes encoding Protein Isoforms involved in neurological disorder. Neurological disorder can be treated (e.g. to ameliorate symptoms or to retard onset or progression) or prevented by administration of a therapeutic compound that promotes function or expression of one or more Protein Isoforms that are decreased in the CSF of subjects having neurological disorder, or by administration of a therapeutic compound that reduces function or expression of one or more Protein Isoforms that are increased in the CSF of subjects having neurological disorder.

In one embodiment, one or more antibodies each specifically binding to a Protein Isoform are administered alone or in combination with one or more additional therapeutic compounds or treatments. Examples of such therapeutic compounds or treatments include, but are not limited to, mood stabilizers: lithium, divalproex, carbamazepine, lamotrigine; antidepressants: tricyclic antidepressants (eg. Desipramine, chlorimipramine, nortriptyline), selective serotonin reuptake inhibitors (SSRIs including fluoxetine (Prozac), sertraltrine (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: Clozapine, Olanzapine, Risperidone. From P31-Sertindole, Haloperidol, Pirenzepine, Perazine, Risperdal, Famotidine, Clozaril, Mesoridazine, Quetiapine, atypical anti-psychotic medications of Risperidone, Zyprexa (Olanzapine) and Clozapine and any other Dibenzothiazepines. Sertindole, Haloperidol, Pirenzepine, Perazine, Risperdal, Famotidine, Clozaril, Mesoridazine, Quetiapine, atypical anti-psychotic medications of Risperidone, Zyprexa (Olanzapine) and Clozapine and any other Dibenzothiazepines, antithrombic therapies such as Danaparoid, Nadroparin and Tinzaparin, thrombolytic and defibrinogenating agents such as Pro-urokinase, streptokinase, tissue plasmoinogen activator and urokinase, antiplatelet agents such as aspirin, Buflomedil (Cucinotta et al. J Int Med Res (1992) 20:136-49), neuroprotective agents such as Propentofylline (Rother et al. Ann N Y Acad Sci (1996) 777:404-9, Mielke et al. Alzheimer Dis Assoc Disord (1998) 12 Suppl 2:S29-35, Rother et al. Dement Geriatr Cogn Disord (1998) 9 Suppl 1:36-43), cholinesterase inhibitors such as rivastigmine, galantamine (Kumar et al. Neurology (1999) 52 Suppl 2:A395) and other cytoprotective agents currently under clinical evaluation such as the calcium antagonists Nimodipine and Nicadipine, NMDA antagonists such as Selfotel, Dextrorphan, Cerestat, Eliprodil, Lamortigine, GABA agonists, Kappa-selective opiod antagonists, Lubeluzole, Free radical scavengers, anti-ICAM antibodies and GM-1 ganglioside, Abbokinase®, Activase®, Aggrenox®, Anti-ICAM-1 antibody, Anti-beta-2-integrin antibody, Arvin®, Atacand®, CerAxon®, Cerebyx®, Ceresine®, Cerestat®, Cervene®, Coumadin®, Fiblast®, Fraxiparine®, Freedox®, Innohep®, Kabikinase®, Klerval®, LeukArrest®, Lipitor®, Lovenox®, Neurogard®, Nimotop®, Orgaran®, Persantine®, Plavix®, Prolyse®, Prosynap®, ReoPro®, Selfotel®, Sibelium®, Streptase®, Streptokinase, Sygen®, Ticlid®, Trental®, Viprinex®, Warfarin, Zanaflex®, Zendra®., tacrine, donepezil, α-tocopherol, selegeline, NSAIDs, estrogen replacement therapy, physostigmine, rivastigmine, hepastigmine, metrifonate, ENA-713, ginkgo biloba extract, physostigmine, amridin, talsaclidine, zifrosilone, eptastigmine, methanesulfonyl chloride, nefiracetam, ALCAR, talsachidine, xanomeline, galanthamine, and propentofylline, Interferon β-1b (Betaseron®, Betaferon®), Interferon β-1a (Avonex®, Rebif®), Glatiramer acetate (Copaxone®), intravenous immunoglobulin and for acute relapse therapies with corticosteroids (Noseworthy (1999) Nature 399:suppl. A40-A47).

Preferably, a biological product such as an antibody is allogeneic to the subject to which it is administered. In a preferred embodiment, a human Protein Isoform or a human Protein Isoform-related polypeptide, a nucleotide sequence encoding a human Protein Isoform or a human Protein Isoform-related polypeptide, or an antibody to a human Protein Isoform or a human Protein Isoform-related polypeptide, is administered to a human subject for therapy (e.g. to ameliorate symptoms or to retard onset or progression) or prophylaxis.

### 5.13.1 Treatment and Prevention of Neurological Disorder

Neurological disorders can be treated or prevented by administration to a subject suspected of having or known to have a neurological disorder or to be at risk of developing a neurological disorder an agent that modulates (i.e., increases or decreases) the level or activity (i.e., function) of one or more Protein Isoforms that are differentially present in the CSF or brain tissue of subjects having a neurological disorder compared with CSF or brain tissue of subjects free from that neurological disorder. In one embodiment, a neurological disorder is treated by administering to a subject suspected of having or known to have a particular neurological disorder or to be at risk of developing a particular neurological disorder an agent that upregulates (i.e., increases) the level or activity (i.e., function) of one or more Protein Isoforms that are decreased in the CSF or brain tissue of subjects having the above neurological disorder. In another embodiment, an agent is administered that downregulates the level or activity (i.e., function) of one or more Protein Isoforms that are increased in the CSF of subjects having a particular neurological disorder. Examples of such a compound include but are not limited to: Protein Isoforms, Protein Isoform fragments and Protein Isoform-related polypeptides; nucleic acids encoding a Protein Isoform, a Protein Isoform fragment and a Protein Isoform-related polypeptide (e.g., for use in gene therapy); and, for those Protein Isoforms or Protein Isoform-related polypeptides with enzymatic activity, compounds or molecules known to modulate that enzymatic activity. Other compounds that can be used, e.g., Protein Isoform agonists, can be identified using in vitro assays, as defined or described above or earlier.

Neurological disorders are also treated or prevented by administration to a subject suspected of having or known to have a neurological disorder or to be at risk of developing a neurological disorder of a compound that downregulates the level or activity of one or more Protein Isoforms that are increased in the CSF or brain tissue of subjects having a neurological disorder. In another embodiment, a compound is administered that upregulates the level or activity of one or more Protein Isoforms that are decreased in the CSF or brain tissue of subjects having a neurological disorder. Examples of such a compound include, but are not limited to, Protein Isoform antisense oligonucleotides, ribozymes, antibodies directed against Protein Isoforms, and compounds that inhibit the enzymatic activity of a Protein Isoform. Other useful compounds e.g., Protein Isoform antagonists and small molecule Protein Isoform antagonists, can be identified using in vitro assays.

In a preferred embodiment, therapy or prophylaxis is tailored to the needs of an individual subject. Thus, in specific embodiments, compounds that promote the level or function of one or more Protein Isoforms are therapeutically or prophylactically administered to a subject suspected of having or known to have neurological disorder, in whom the levels or functions of said one or more Protein Isoforms are absent or are decreased relative to a control or normal reference range. In further embodiments, compounds that promote the level or function of one or more Protein Isoforms are therapeutically or prophylactically administered to a subject suspected of having or known to have neurological disorder in whom the levels or functions of said one or more Protein Isoforms are increased relative to a control or to a reference range. In further embodiments, compounds that decrease the level or function of one or more Protein Isoforms are therapeutically or prophylactically administered to a subject suspected of having or known to have neurological disorder in whom the levels or functions of said one or more Protein Isoforms are increased relative to a control or to a reference range. In further embodiments, compounds that decrease the level or function of one or more Protein Isoforms are therapeutically or prophylactically administered to a subject suspected of having or known to have a neurological disorder in whom the levels or functions of said one or more Protein Isoforms are decreased relative to a control or to a reference range. The change in Protein Isoform function or level due to the administration of such compounds can be readily detected, e.g., by obtaining a sample (e.g., a sample of CSF, blood or urine or a tissue sample such as brain biopsy tissue) and assaying in vitro the levels or activities of said Protein Isoforms, or the levels of mRNAs encoding said Protein Isoforms or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the neurological disorder Protein Isoform profile towards normal with the proviso that such compounds do not include—lithium, divalproex, carbamazepine, lamotrigine; antidepressants: tricyclic antidepressants (eg. Desipramine, chlorimipramine, nortriptyline), selective serotonin reuptake inhibitors (SSRIs including fluoxetine (Prozac), sertraltrine (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: Clozapine, Olanzapine, Risperidone. Haloperidol, Pirenzepine, Perazine, Risperdal, Famotidine, Clozaril, Mesoridazine, Quetiapine, atypical anti-psychotic medications of Risperidone, Zyprexa (Olanzapine) and Clozapine and any other Dibenzothiazepines. Danaparoid, Nadroparin and Tinzaparin, thrombolytic and defibrinogenating agents such as Pro-urokinase, streptokinase, tissue plasmoinogen activator and urokinase, antiplatelet agents such as aspirin, Buflomedil (Cucinotta et al. J Int Med Res (1992) 20:136-49), neuroprotective agents such as Propentofylline (Rother et al. Ann N Y Acad Sci (1996) 777:404-9, Mielke et al. Alzheimer Dis Assoc Disord (1998) 12 Suppl 2:S29-35, Rother et al. Dement Geriatr Cogn Disord (1998) 9 Suppl 1:36-43), cholinesterase inhibitors such as rivastigmine, galantamine (Kumar et al. Neurology (1999) 52 Suppl 2:A395) and other cytoprotective agents currently under clinical evaluation such as the calcium antagonists Nimodipine and Nicadipine, NMDA antagonists such as Selfotel, Dextrorphan, Cerestat, Eliprodil, Lamortigine, GABA agonists, Kappa-selective opiod antagonists, Lubeluzole, Free radical scavengers, anti-ICAM antibodies and GM-1 ganglioside, Abbokinase®, Activase®, Aggrenox®, Anti-ICAM-1 antibody, Anti-beta-2-integrin antibody, Arvin®, Atacand®, CerAxon®, Cerebyx®, Ceresine®, Cerestat®, Cervene®, Coumadin®, Fiblast®, Fraxiparine®, Freedox®, Innohep®, Kabikinase®, Klerval®, LeukArrest®, Lipitor®, Lovenox®, Neurogard®, Nimotop®, Orgaran®, Persantine®, Plavix®, Prolyse®, Prosynap®, ReoPro®, Selfotel®, Sibelium®, Streptase®, Streptokinase, Sygen®, Ticlid®, Trental®, Viprinex®, Warfarin, Zanaflex®, Zendra®, tacrine, donepezil, rivastigmine, hepastigmine, Metrigonate, physostigmine, Amridin, Talsaclidine, KA-672, Huperzine, P-11012, P-11149, Zifrosilone, Eptastigmine, Methanesulfonyl chloride, and S-9977), an acetylcholine receptor agonist (e.g., Nefiracetam, LU-25109, and NS2330), a muscarinic receptor agonist (e.g., SB-20206, Talsachidine, ADF-1025B, and SR-46559A), a nicotonic cholinergic receptor agonist (e.g., ABT-418), an acetylcholine modulator (e.g., FKS-508 and Galantamine) or propentofylline, Interferon β-1b (Betaseron®, Betaferon®), Interferon β-1a (Avonex®, Rebif®), Glatiramer acetate (Copaxone®), intravenous immunoglobulin and for acute relapse therapies with corticosteroids (Noseworthy (1999) Nature 399:suppl. A40-A47).

### 5.13.2 Gene Therapy

In another embodiment, nucleic acids comprising a sequence encoding a Protein Isoform, a Protein Isoform fragment, Protein Isoform-related polypeptide or fragment of a Protein Isoform-related polypeptide, are administered to promote Protein Isoform function by way of gene therapy. Gene therapy refers to the administration of an expressed or expressible nucleic acid to a subject. In this embodiment, the nucleic acid produces its encoded polypeptide and the polypeptide mediates a therapeutic effect by promoting Protein Isoform function.

Any suitable methods for gene therapy available in the art can be used according to the present invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a particular aspect, the compound comprises a nucleic acid encoding a Protein Isoform or fragment or chimeric protein thereof, said nucleic acid being part of an expression vector that expresses a Protein Isoform or fragment or chimeric protein thereof in a suitable host. In particular, such a nucleic acid has a promoter operably linked to the Protein Isoform coding region, said promoter being inducible or constitutive (and, optionally, tissue-specific). In another particular embodiment, a nucleic acid molecule is used in which the Protein Isoform coding sequences and any other desired sequences are flanked by regions that promote homologous recombination at a desired site in the genome, thus providing for intrachromosomal expression of the Protein Isoform nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a further embodiment, a viral vector that contains a nucleic acid encoding a Protein Isoform is used, for example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the Protein Isoform to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a Protein Isoform is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid to be introduced for purposes of gene therapy may comprise an inducible promoter operably linked to the coding region, such that expression of the nucleic acid is controllable by controlling the presence or absence of the appropriate inducer of transcription.

Direct injection of a DNA coding for a Protein Isoform may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a Protein Isoform and (b) a promoter are injected into a subject to elicit an immune response to the Protein Isoform.

### 5.13.3 Inhibition of Protein Isoforms to Treat a Neurological Disorder

In one embodiment of the invention, a neurological disorder is treated or prevented by administration of a compound that antagonizes (inhibits) the level(s) and/or function(s) of one or more Protein Isoforms which are elevated in the CSF of subjects having a neurological disorder as compared with CSF of subjects free from the above neurological disorder. Compounds useful for this purpose include but are not limited to anti-Protein Isoform antibodies (and fragments and derivatives containing the binding region thereof), Protein Isoform antisense or ribozyme nucleic acids, and nucleic acids encoding dysfunctional Protein Isoforms that are used to “knockout” endogenous Protein Isoform function by homologous recombination (see, e.g., Capecchi, 1989, Science 244:1288-1292). Other compounds that inhibit Protein Isoform function can be identified by use of known in vitro assays, e.g., assays for the ability of a test compound to inhibit binding of a Protein Isoform to another protein or a binding partner, or to inhibit a known Protein Isoform function. Preferably such inhibition is assayed in vitro or in cell culture, but genetic assays may also be employed. The Preferred Technology can also be used to detect levels of the Protein Isoform before and after the administration of the compound. Preferably, suitable in vitro or in vivo assays are utilized to determine the effect of a specific compound and whether its administration is indicated for treatment of the affected tissue, as described in more detail below.

In a particular embodiment, a compound that inhibits a Protein Isoform function is administered therapeutically or prophylactically to a subject in whom an increased CSF level or functional activity of the Protein Isoform (e.g., greater than the normal level or desired level) is detected as compared with CSF of subjects free from neurological disorder or a predetermined reference range. Methods standard in the art can be employed to measure the increase in a Protein Isoform level or function, as outlined above. Preferred Protein Isoform inhibitor compositions include small molecules, i.e., molecules of 1000 daltons or less. Such small molecules can be identified by the screening methods described herein.

### 5.13.4 Antisense Regulation of Protein Isoforms

In a further embodiment, Protein Isoform expression is inhibited by use of Protein Isoform antisense nucleic acids. The present invention provides the therapeutic or prophylactic use of nucleic acids comprising at least six nucleotides that are antisense to a gene or cDNA encoding a Protein Isoform or a portion thereof. As used herein, a Protein Isoform “antisense” nucleic acid refers to a nucleic acid capable of hybridizing by virtue of some sequence complementarity to a portion of an RNA (preferably mRNA) encoding a Protein Isoform. The antisense nucleic acid may be complementary to a coding and/or noncoding region of an mRNA encoding a Protein Isoform. Such antisense nucleic acids have utility as compounds that inhibit Protein Isoform expression, and can be used in the treatment or prevention of neurological disorder.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of a Protein Isoform antisense nucleic acid, and a pharmaceutically-acceptable carrier, vehicle or diluent.

In another embodiment, the invention provides methods for inhibiting the expression of a Protein Isoform nucleic acid sequence in a prokaryotic or eukaryotic cell comprising providing the cell with an effective amount of a composition comprising a Protein Isoform antisense nucleic acid of the invention.

Protein Isoform antisense nucleic acids and their uses are described in detail below.

### 5.13.5 Protein Isoform Antisense Nucleic Acids

The Protein Isoform antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a particular aspect of the invention, a Protein Isoform antisense oligonucleotide is provided, preferably of single-stranded DNA. The oligonucleotide may be modified at any position on its structure with substituents generally known in the art.

The Protein Isoform antisense oligonucleotide may comprise any suitable of the following modified base moieties, e.g., 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, bcta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an, α-anomeric oligonucleotide. An, α-anomeric oligonucleotide forms specific double-stranded hybrids with complementary RNA in which, contrary to the usual, β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In another embodiment, the Protein Isoform antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the Protein Isoform antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the Protein Isoform antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a Protein Isoform, preferably a human gene encoding a Protein Isoform, however, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.). with the RNA, forming a stable duplex; in the case of double-stranded Protein Isoform antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding a Protein Isoform it may contain and still form a stable duplex (or triplex, as the case may be). One skilled in the art can ascertain a tolerable degree of mismatch by use of standard procedures to determine the melting point of the hybridized complex.

### 5.13.6 Therapeutic Use of Protein Isoform Antisense Nucleic Acids

The Protein Isoform antisense nucleic acids can be used to treat or prevent neurological disorder, when the target Protein Isoform is overexpressed in the CSF of subjects suspected of having or suffering from neurological disorder. In a preferred embodiment, a single-stranded DNA antisense Protein Isoform oligonucleotide is used.

Cell types which express or overexpress RNA encoding a Protein Isoform can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with a Protein Isoform-specific nucleic acid (e.g., by Northern hybridization, dot blot hybridization, in situ hybridization), observing the ability of RNA from the cell type to be translated in vitro into a Protein Isoform, immunoassay, etc. In a preferred aspect, primary tissue from a subject can be assayed for Protein Isoform expression prior to treatment, e.g., by immunocytochemistry or in situ hybridization.

Pharmaceutical compositions of the invention, comprising an effective amount of a Protein Isoform antisense nucleic acid in a pharmaceutically acceptable carrier, vehicle or diluent can be administered to a subject having neurological disorder.

The amount of Protein Isoform antisense nucleic acid which will be effective in the treatment of a neurological disorder can be determined by standard clinical techniques.

In a specific embodiment, pharmaceutical compositions comprising one or more Protein Isoform antisense nucleic acids are administered via liposomes, microparticles, or microcapsules. In various embodiments of the invention, such compositions may be used to achieve sustained release of the Protein Isoform antisense nucleic acids.

### 5.13.7 Inhibitory Ribozyme and Triple Helix Approaches

In another embodiment, symptoms of neurological disorder may be ameliorated by decreasing the level of a Protein Isoform or Protein Isoform activity by using gene sequences encoding the Protein Isoform in conjunction with well-known gene “knock-out,” ribozyme or triple helix methods to decrease gene expression of a Protein Isoform. In this approach ribozyme or triple helix molecules are used to modulate the activity, expression or synthesis of the gene encoding the Protein Isoform, and thus to ameliorate the symptoms of neurological disorder. Such molecules may be designed to reduce or inhibit expression of a mutant or non-mutant target gene. Techniques for the production and use of such molecules are well known to those of skill in the att.

Ribozyme molecules designed to catalytically cleave gene mRNA transcripts encoding a Protein Isoform can be used to prevent translation of target gene mRNA and, therefore, expression of the gene product (See, e.g., PCT International Publication WO90/11364, published Oct. 4, 1990; Sarver et al., 1990, Science 247:1222-1225).

Ribozymes are enzymatic RNA molecules capable of catalyzing the specific cleavage of RNA (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding a Protein Isoform, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the Protein Isoform, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the Protein Isoform.

As in the antisense approach, the ribozymes can be composed of modified oligonucleotides (e.g., for improved stability, targeting, etc.) and should be delivered to cells that express the Protein Iso form in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the Protein Isoform and inhibit translation. Because ribozymes, unlike antisense molecules, are catalytic, a lower intracellular concentration is required for efficacy.

Endogenous Protein Isoform expression can also be reduced by inactivating or “knocking out” the gene encoding the Protein Isoform, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional Protein Isoform (or a completely unrelated DNA sequence) flanked by DNA homologous to the endogenous gene (either the coding regions or regulatory regions of the gene encoding the Protein Isoform) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However, this approach can be adapted for use in humans provided the recombinant DNA constructs are directly administered or targeted to the required site in vivo using appropriate viral vectors.

Alternatively, the endogenous expression of a gene encoding a Protein Isoform can be reduced by targeting deoxyribonucleotide sequences complementary to the regulatory region of the gene (i.e., the gene promoter and/or enhancers) to form triple helical structures that prevent transcription of the gene encoding the Protein Isoform in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription in the present invention should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

In one embodiment, wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of a Protein Isoform that the situation may arise wherein the concentration of Protein Isoform present may be lower than is necessary for a normal phenotype. In such cases, to ensure that substantially normal levels of activity of a gene encoding a Protein Isoform are maintained, gene therapy may be used to introduce into cells nucleic acid molecules that encode and express the Protein Isoform that exhibit normal gene activity and that do not contain sequences susceptible to whatever antisense, ribozyme, or triple helix treatments are being utilized. Alternatively, in instances whereby the gene encodes an extracellular protein, normal Protein Isoform can be co-administered in order to maintain the requisite level of Protein Isoform activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### 5.14 Assays for Therapeutic or Prophylactic Compounds

The present invention also provides assays for use in discovery of pharmaceutical products in order to identify or verify the efficacy of compounds for treatment or prevention of neurological disorder. Agents can be assayed for their ability to restore Protein Isoform levels in a subject having neurological disorder towards levels found in subjects free from neurological disorder or to produce similar changes in experimental animal models of neurological disorder. Compounds able to restore Protein Isoform levels in a subject having neurological disorder towards levels found in subjects free from neurological disorder or to produce similar changes in experimental animal models of neurological disorder can be used as lead compounds for further drug discovery, or used therapeutically. Protein Isoform expression can be assayed by the Preferred Technology, immunoassays, gel electrophoresis followed by visualization, detection of Protein Isoform activity, or any other method taught herein or known to those skilled in the art. Such assays can be used to screen candidate drugs, in clinical monitoring or in drug development, where abundance of a Protein Isoform can serve as a surrogate marker for clinical disease.

In various embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Examples of animal models of neurological disorder include, but are not limited to: animal models of depression e.g animals that express human familial BAD genes and the Porsolt forced swim test model of depression is frequently used in both these contexts (Kirby and Lucki, 1997; Rossetti et al., 1993). The two major clinical states observed in bipolar disorder (depression and mania) have also been successfully modeled (Cappeliez and Moore Prog Neuropsychopharmacol Biol Psychiatry 1990 14, 347-58). Psychostimulant treatment can produce a range of behaviors similar to that of mania including hyperactivity, heightened sensory awareness, and alertness, and for this reason has become a very useful model for mania which exhibits (to some extent) face, construct and predictive validity. Another model that has been utilized for the development of bipolar illness is behavioral sensitization. In this model, the repeated administration of many psychostimulant drugs leads to a gradual increase or sensitization of the drug-induced behavioral; this model also has considerable construct and face validity for mania (Koob et al. Pharmacol Biochem Behav 1997 57, 513-21). Animal models of Schizophrenia: Phencyclidine treated rodents (Sams-Dodd Rev Neurosci (1999) 10:59-90), an animal model of deficient sensorimotor gating (Swerdlow and Geyer Schizophr Bull (1998) 24(2):285-301), neonatal insult to the hippocampal region (Beauregard and Bachevalier Can J Psychiatry (1996) September 41(7):446-56), models based on neonatal excitotoxic hippocampal damage (Lillrank et al, Clin Neurosci (1995) 3(2):98-104), attention deficit models (Feldon et al, J Psychiatr Res 4:345-66) and NMDA deficient rodent models (Mohn et al, Cell 1999, 98, 427-436), animals that show decreased expression of mRNAs for synaptophysin, GAP-43, cholecystokinin, and non-NMDA glutamate receptor subunits (GLU R 1 and 2), particularly in CA 3-4 associated with Schizophrenia (Weinberger Biol Psychiatry (1999) February 15 45:4 395-402) can be utilized to test compounds that modulate Protein Isoform levels, since the neuropathology exhibited in these models is similar to that of Schizophrenia. Animal models of vascular dementia: including models of cerebral beta-amyloid angiopathy (Walker, Brain Res. Rev. (1997) 25:70-84), animal models of vascular dementia with emphasis on stroke-prone spontaneously hypertensive rats (Saito et al., Clin. Exp. Pharmacol. Physiol. (1995) 22 Suppl 1:s257-259), unilateral middle cerebral artery (MCA) occlusion, multiple small embolization or transient four-vessel occlusion model rats producing acute single or multi-infarct dementia memory impairments (Naritomi, Alzheimer Dis Assoc Disord (1991) 5:103-11), stroke-prone spontaneously hypertensive rats (Togashi et al, Stroke (1996) 27:520-5; discussion 525-6), Icelandic-like mutation of Cystatin C in an animal model of cerebrovascular beta-amyloidosis (Wei, Stroke (1996) 27:2080-5). Moreover, common models of stroke have been reviewed by Feuerstein and Wang (Feuerstein G Z, Wang X, Animal models of stroke, Mol Med Today 2000 March; 6(3):133-5). Such animal models can be utilized to test compounds that modulate Protein Isoform levels, since the neuropathology exhibited in these models is similar to that of Vascular Dementia Animal models of Alzheimer's disease: animals that express human familial Alzheimer's disease (FAD) β-amyloid precursor (APP), animals that overexpress human wild-type APP, animals that overexpress β-amyloid 1-42 (11A), animals that express FAD presenillin-1 (PS-1) (see, e.g., Higgins, L S, 1999, Molecular Medicine Today 5:274-276). Further, animal models for Downs syndrome (e.g., TgSOD1, TgPFKL, TgS100β, TgAPP, TgEts2, TgHMG14, TgMNB, Ts65Dn, and Ts1Cje (see, e.g., Kola et al., 1999, Molecular Medicine Today 5:276-277) can be utilized to test compounds that modulate Protein Isoform levels since the neuropathology exhibited by individuals with Downs syndrome is similar to that of Alzheimer's disease. Animal models of Multiple Sclerosis: experimental autoimmune encephalomyelitis (EAE) (Steinman (1999) Neuron, 24:511-514). It is also apparent to the skilled artisan that, based upon the present disclosure, transgenic animals can be produced with “knock-out” mutations of the gene or genes encoding one or more Protein Isoforms. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of a Protein Isoform are identified in non-human animals (e.g., mice, rats, monkeys, rabbits, and guinea pigs), preferably non-human animal models for neurological disorder, expressing the Protein Isoform. In accordance with this embodiment, a test compound or a control compound is administered to the animals, and the effect of the test compound on expression of one or more Protein Isoforms is determined. A test compound that alters the expression of a Protein Isoform (or a plurality of Protein Isoforms) can be identified by comparing the level of the selected Protein Isoform or Protein Isoforms (or mRNA(s) encoding the same) in an animal or group of animals treated with a test compound with the level of the Protein Isoform(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of a Protein Isoform or a biologically active portion thereof are identified in non-human animals (e.g., mice, rats, monkeys, rabbits, and guinea pigs), preferably non-human animal models for neurological disorder, expressing the Protein Isoform. In accordance with this embodiment, a test compound or a control compound is administered to the animals, and the effect of a test compound on the activity of a Protein Isoform is determined. A test compound that alters the activity of a Protein Isoform (or a plurality of Protein Isoforms) can be identified by assaying animals treated with a control compound and animals treated with the test compound. The activity of the Protein Isoform can be assessed by detecting induction of a cellular second messenger of the Protein Isoform (e.g., intracellular diacylglycerol, IP3, etc.), detecting catalytic or enzymatic activity of the Protein Isoform or binding partner thereof, detecting the induction of a reporter gene (e.g., a regulatory element that is responsive to a Protein Isoform of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a Protein Isoform (see, e.g., U.S. Pat. No. 5,401,639, which is incorporated herein in its entirety by reference).

In yet another embodiment, test compounds that modulate the level or expression of a Protein Isoform (or plurality of Protein Isoforms) are identified in human subjects having neurological disorder, most preferably those having severe neurological disorder. In accordance with this embodiment, a test compound or a control compound is administered to the human subject, and the effect of a test compound on Protein Isoform expression is determined by analyzing the expression of the Protein Isoform or the mRNA encoding the same in a biological sample (e.g., CSF, serum, plasma, or urine). A test compound that alters the expression of a Protein Isoform can be identified by comparing the level of the Protein Isoform or mRNA encoding the same in a subject or group of subjects treated with a control compound to that in a subject or group of subjects treated with a test compound. Alternatively, alterations in the expression of a Protein Isoform can be identified by comparing the level of the Protein Isoform or mRNA encoding the same in a subject or group of subjects before and after the administration of a test compound. Any suitable techniques known to those of skill in the art can be used to obtain the biological sample and analyze the mRNA or protein expression. For example, the Preferred Technology described herein can be used to assess changes in the level of a Protein Isoform.

In another embodiment, test compounds that modulate the activity of a Protein Isoform (or plurality of Protein Isoforms) are identified in human subjects having neurological disorder, most preferably those with severe neurological disorder. In this embodiment, a test compound or a control compound is administered to the human subject, and the effect of a test compound on the activity of a Protein Isoform is determined. A test compound that alters the activity of a Protein Isoform can be identified by comparing biological samples from subjects treated with a control compound to samples from subjects treated with the test compound. Alternatively, alterations in the activity of a Protein Isoform can be identified by comparing the activity of a Protein Isoform in a subject or group of subjects before and after the administration of a test compound. The activity of the Protein Isoform can be assessed by detecting in a biological sample (e.g., CSF, serum, plasma, or urine) induction of a cellular signal transduction pathway of the Protein Isoform (e.g., intracellular Ca2+, diacylglycerol, IP3, etc.), catalytic or enzymatic activity of the Protein Isoform or a binding partner thereof, or a cellular response, for example, cellular differentiation, or cell proliferation. Techniques known to those of skill in the art can be used to detect changes in the induction of a second messenger of a Protein Isoform or changes in a cellular response. For example, RT-PCR can be used to detect changes in the induction of a cellular second messenger.

In a particular embodiment, an agent that changes the level or expression of a Protein Isoform towards levels detected in control subjects (e.g., humans free from neurological disorder) is selected for further testing or therapeutic use. In another preferred embodiment, a test compound that changes the activity of a Protein Isoform towards the activity found in control subjects (e.g., humans free from neurological disorder) is selected for further testing or therapeutic use.

In another embodiment, test compounds that reduce the severity of one or more symptoms associated with neurological disorder are identified in human subjects having neurological disorder, most preferably subjects with severe neurological disorder. In accordance with this embodiment, a test compound or a control compound is administered to the subjects, and the effect of a test compound on one or more symptoms of neurological disorder is determined. A test compound that reduces one or more symptoms can be identified by comparing the subjects treated with a control compound to the subjects treated with the test compound. Techniques known to physicians familiar with neurological disorder can be used to determine whether a test compound reduces one or more symptoms associated with neurological disorder. For example, a test compound that improves cognitive ability in a subject having Alzheimer's disease or Vascular dementia will be beneficial for treating subjects having these neurological disorders.

In a preferred embodiment, an agent that reduces the severity of one or more symptoms associated with a neurological disorder in a human having such a neurological disorder is selected for further testing or therapeutic use.

### 5.15 Therapeutic and Prophylactic Compositions and their Use

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into CSF or at the site (or former site) of neurodegeneration or to CNS tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other suitable controlled release systems are discussed in the review by Langer (1990, Science 249:1527-1533).

In another embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to case pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of neurological disorder can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### 5.16 Determining Abundance of Protein Isoforms by Imaging Technology

An advantage of determining abundance of Protein Isoforms by imaging technology may be that such a method is non-invasive (save that reagents may need to be administered) and there is no need to extract a sample from the subject.

Suitable imaging technologies include positron emission tomography (PET) and single photon emission computed tomography (SPECT). Visualisation of the Protein Isoforms using such techniques requires incorporation or binding of a suitable label eg a radiotracer such as ¹⁸F, ¹¹C or ¹²³I (see eg NeuroRx—The Journal of the American Society for Experimental NeuroTherapeutics (2005) 2(2), 348-360 and idem pages 361-371 for further details of the techniques). Radiotracers or other labels may be incorporated into the Protein Isoforms by administration to the subject (eg by injection) of a suitably labelled specific ligand. Alternatively they may be incorporated into a binding affinity reagent (antibody, Affibody etc) specific for the Protein Isoform which may be administered to the subject (eg by injection). For discussion of use of Affibodies for imaging see eg Orlova A, Magnusson M, Eriksson T L, Nilsson M, Larsson B, Hoiden-Guthenberg I, Widstrom C, Carlsson J, Tolmachev V, Stahl S, Nilsson F Y, Tumor imaging using a picomolar affinity HER2 binding affibody molecule, Cancer Res. 2006 Apr. 15; 66(8):4339-48).

### 6. Example

### Identification of Proteins Differentially Expressed in the CSF of Neurological Disorder Patients

Using the following exemplary and non-limiting procedure, proteins in CSF samples from subjects having various neurological disorder and control subjects were separated, by isoelectric focusing followed by SDS-PAGE, and analyzed. Parts 6.1.1 to 6.1.19 (inclusive) of the procedure set forth below are hereby designated as the “Reference Protocol”.

### 6.1 Materials and Methods

### 6.1.1 Sample Preparation

A protein assay (Pierce BCA Cat # 23225) was performed on each CSF sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from CSF (“CSF depletion”) was achieved by an affinity chromatography purification step in which the sample was passed through a series of >Hi-Trap′ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted CSF containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2×)

This mixture was vortexed, and centrifuged at 13000 rpm for 5 mins at 15° C., and the supernatant was separated by isoelectric focusing as described below.

### 6.1.2 Isoelectric Focusing

Isoelectric focusing (IEF), was performed using the Immobiline-7 DryStrip Kit (Pharmacia BioTech), following the procedure described in the manufacturer's instructions, see Instructions for Immobiline-7 DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 501 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5 W. The temperature was held at 20° C. throughout the run.

### 6.1.3 Gel Equilibration and SDS-PAGE

After the final 19 hr step, the ships were immediately removed and immersed for 10 mins at 20° C. in a first solution of the following composition: 6M urea; 2% (w/v) DTT; 2% (w/v) SDS; 30% (v/v) glycerol (Fluky 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

### 6.1.4 Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilaneJ; Pharmacia Cat. # 17-1330-01). The front plate was treated with (RepelSilaneJ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The front and back plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored individually at 4° C. in sealed polyethylene bags with 6 ml of gel buffer, and were used within 4 weeks.

### 6.1.5 SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150 W, for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

### 6.1.6 Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Sypro Red (Molecular Probes, Inc., Eugene, Oreg.). Alternative dyes which can be used for this purpose are described in U.S. patent application Ser. No. 09/412,168, filed Oct. 5, 1999, and incorporated herein by reference in its entirety.

### 6.1.7 Imaging of the gel

A computer-readable output was produced by imaging the fluorescently stained gels with the Apollo 2 scanner (Oxford Glycosciences, Oxford, UK) described in section 5.1, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

### 6.1.8 Digital Analysis of the Data

The data were processed as described in U.S. Pat. No. 6,064,654, (published as WO 98/23950) at Sections 5.4 and 5.5 (incorporated herein by reference), as set forth more particularly below.

The output from the scanner was first processed using the MELANTE7 II 2D PAGE analysis program (Release 2.2, 1997, BioRad Laboratories, Hercules, Calif., Cat # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

### 6.1.9 Assignment of pI and MW Values

Landmark identification was used to determine the pI and MW of features detected in the images. Sixteen landmark features, designated CSFL1 to CSFL16, were identified in a standard CSF image. These landmark features were assigned the pI and/or MW values identified in Table V.

TABLE V
Landmark Features Used in this Study

| Landmark Name | pI | Mw |
|---|---|---|
| CSFL-1 | — | 185225 |
| CSFL-2 | 5.39 | 141699 |
| CSFL-3 | 6.29 | 100728 |
| CSFL-4 | 5.06 | 71271 |
| CSFL-5 | 7.68 | 68368 |
| CSFL-6 | 5.67 | 48092 |
| CSFL-7 | 4.78 | 41342 |
| CSFL-8 | 9.2 | 39998 |
| CSFL-9 | 5.5 | 31894 |
| CSFL-10 | 6.94 | 27439 |
| CSFL-11 | 5.9 | 23992 |
| CSFL-12 | 4.28 | 21372 |
| CSFL-13 | 8.92 | 16019 |
| CSFL-14 | 4.66 | 14570 |
| CSFL-15 | 6.43 | 10961 |
| CSFL-16 | 4.22 | — |

As many of these landmarks as possible were identified in each gel image of the dataset. Each feature in the study gels was then assigned a pI value by linear interpolation or extrapolation (using the MELANIE7-II software) to the two nearest landmarks, and was assigned a MW value by linear interpolation or extrapolation (using the MELANIE7-II software) to the two nearest landmarks.

### 6.1.10 Matching With Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

### 6.1.11 Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE7 II 2D PAGE analysis program was used to calculate and record approximately 500-700 matched feature pairs between the primary master and each of the other images. The accuracy of, this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

### 6.1.12. Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the Protein Isoforms, 4) the apparent molecular weight (MW) of the Protein Isoforms, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient

### 6.1.13. Statistical Analysis of the Profiles

The statistical strategies specified below were used in the order in which they are listed to identify Protein Isoforms from the MCIs within the mastergroup.
a) A percentage feature presence was calculated across the control samples and the CSF samples for each MCI that was a potential Protein Isoform. The MCI was required to be present in at least 20% of samples from a neurological disorder or in at least 20% of the samples from the age-matched control group. The MCIs which fulfilled these criteria were then subjected to further analysis
b) The percentage feature presence for each remaining MCI was then further examined and the MCIs were divided into 3 groups—those which had at least 20% feature presence in a neurological disorder sample group but were absent from all samples in the control group (designated 4+), those with at least 20% feature presence in the control sample group but which were absent from all samples in the neurological disorder sample group (designated 4-) and those MCIs which were present in both disease and control sample groups. The MCIs that were present in both sample groups were subjected to further analysis.
c) A second selection strategy for the MCIs remaining from (b) was based on the fold change. A fold change representing the ratio of the average normalized protein abundances of the Protein Isoforms within an MCI, was calculated for each MCI between each the neurological disorder samples and its age-matched set of controls. A minimum threshold of 1.5 was set for the fold change to be considered significant. Those with a fold change less than 1.5 were designated as “1”, those with a fold change greater than 1.5 were subjected to further analysis
d) For the final selection strategy the Wilcoxon Rank-Sum test was used. This test was performed between the control and the neurological disorder. samples for each MCI with a fold change greater than 1.5. The MCIs which recorded a p-value less than or equal to 0.05 were selected as statistically significant Protein Isoforms with 95% selectivity. The MCIs with a statistically significant change (p<0.05) were designated “3” and the MCIs which did not reach statistical significance were designated “2”. For the latter two groups a “+” or “−” sign was used to indicate a fold increase or a fold decrease respectively.

Application of these four analysis strategies allowed Protein Isoforms to be selected on the basis of: (a) feature presence in at least 20% of samples from control subjects or patients with a neurological disorders (b) qualitative differences with a chosen selectivity, (c) a significant fold change above a threshold with a chosen selectivity or (d) statistically significant changes as measured by the Wilcoxon Rank-Sum test

### 6.1.14 Recovery and Analysis of Selected Proteins

Protein Isoforms were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.), equipped with a Nanoflow™ electrospray Z-spray source. For partial amino acid sequencing and identification of Protein Isoforms uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

### 6.1.15 Discrimination of Neurological Disorder-Associated Proteins

The process to identify the Protein Isoforms of the invention uses the peptide sequences obtained experimentally by mass spectrometry described above of naturally occurring human proteins to identify and organize coding exons in the published human genome sequence.

Recent dramatic advances in defining the chemical sequence of the human genome have led to the near completion of this immense task (Venter, J. C. et al. (2001). The sequence of the human genome. Science 16: 1304-51; International Human Genome Sequencing Consortium. (2001). Initial sequencing and analysis of the human genome Nature 409: 860-921). There is little doubt that this sequence information will have a substantial impact on our understanding of many biological processes, including molecular evolution, comparative genomics, pathogenic mechanisms and molecular medicine. For the full medical value inherent in the sequence of the human genome to be realised, the genome needs to be ‘organised’ and annotated. By this, is meant at least the following three things. (i) The assembly of the sequences of the individual portions of the genome into a coherent, continuous sequence for each chromosome. (ii) The unambiguous identification of those regions of each chromosome that contain genes. (iii) Determination of the fine structure of the genes and the properties of its mRNA and protein products. While the definition of a ‘gene’ is an increasingly complex issue (H Pearson: What is a gene? Nature (2006) 24: 399-401)), what is of immediate interest for drug discovery and development is a catalogue of those genes that encode functional, expressed proteins. A subset of these genes will be involved in the molecular basis of most if not all pathologies. Therefore an important and immediate goal for the pharmaceutical industry is to identify all such genes in the human genome and describe their fine structure.

### Processing and Integration of Peptide Masses, Peptide Signatures, ESTs and Public Domain Genomic Sequence Data to Form OGAP® Database

Discrete genetic units (exons, transcripts and genes) were identified using the following sequential steps:
1. A ‘virtual transcriptome’ is generated, containing the tryptic peptides which map to the human genome by combining the gene identifications available from Ensembl and various gene prediction programs. This also incorporates SNP data (from dbSNP) and all alternate splicing of gene identifications. Known contaminants were also added to the virtual transcriptome.
2. All tandem spectra in the OGeS Mass Spectrometry Database are interpreted in order to produce a peptide that can be mapped to one in the virtual transcriptome. A set of automated spectral interpretation algorithms were used to produce the peptide identifications.
3. The set of all mass-matched peptides in the OGeS Mass Spectrometry Database is generated by searching all peptides from transcripts hit by the tandem peptides using a tolerance based on the mass accuracy of the mass spectrometer, typically 20 ppm.
4. All tandem and mass-matched peptides are combined in the form of “protein clusters”. This is done using a recursive process which groups sequences into clusters based on common peptide hits. Biological sequences are considered to belong to the same cluster if they share one or more tandem or mass-matched peptide.
5. After initial filtering to screen out incorrectly identified peptides, the resulting clusters are then mapped on the human genome.
6. The protein clusters are then aggregated into regions that define preliminary gene boundaries using their proximity and the co-observation of peptides within protein clusters. Proximity is defined as the peptide being within 80,000 nucleotides on the same strand of the same chromosome. Various elimination rules, based on cluster observation scoring and multiple mapping to the genome are used to refine the output. The resulting ‘confirmed genes’ are those which best account for the peptides and masses observed by mass spectrometry in each cluster. Nominal co-ordinates for the gene are also an output of this stage.
7. The best set of transcripts for each confirmed gene are created from the protein clusters, peptides, ESTs, candidate exons and molecular weight of the original protein spot
8. Each identified transcript is linked to the sample providing the observed peptides
9. Use of an application for viewing and mining the data. The result of steps 1-8 is a database containing genes, each of which consists of a number of exons and one or more transcripts. An application was written to display and search this integrated genome/proteome data. Any features (OMIM disease locus, InterPro etc.) that had been mapped to the same Golden Path co-ordinate system by Ensembl could be cross-referenced to these genes by coincidence of location and fine structure.

### Results

The process was used to generate approximately 1 million peptide sequences to identify protein-coding genes and their exons resulted in the identification of protein sequences for 18083 genes across 67 different tissues and 57 diseases including 2306 genes in Alzheimer's disease, 260 genes in Depression, 173 genes in Multiple Sclerosis, 130 genes in Schizophrenia and 51 genes in Vascular Dementia. Following comparison of the experimentally determined sequences with sequences in the OGAP® database, the Protein Isoforms listed in the Table I herein showed a high degree of specificity to neurological disorders indicative of the prognostic and diagnostic nature.

### 6.2 Results

These initial experiments identified thirty six Protein Isoforms, of which 7 were altered in Alzheimer's disease, schizophrenia, bipolar depression, multiple sclerosis and vascular dementia as compared with normal. Their details are given in Table 1. They all belong to one of the three Protein Isoform Families as defined supra, the mature protein (PIF-1), the C-terminal part of the protein (PIF-2), or the N-terminal part of the protein (PIE-3). The PIF-1 members have been found to be processed proteolytically in the presence of neurological disorders, such that the PIF-2 and PIF-3 members have been found to be generally upregulated in the presence of neurological disorders. See also FIGS. 3 and 4.

### 7. Example

### Identification of Glycosylation Sites on Ex Vivo Protein Isoforms of the Invention

As mentioned above, different isoforms within one PIF family will differ by the length of their primary sequence, and also by the post-translational modifications they have. The protocol detailed below allows the precise characterization of the glycosylation sites present on a given Protein Isoform.

### 7.1 Glycocapture—LC-MS

The sample is changed to coupling buffer (100 mM NaAc, pH 5.5, and 150 mM NaCl) using an Econo-Pac10DG desalting column (Bio-Rad, Hercules Calif.), equilibrated to a final concentration of 15 mM sodium periodate and incubated at room temperature for 1 hour. Using the same Econo-Pac10DG desalting column the sodium periodate is removed from the sample and hydrazide resin equilibrated in coupling buffer is added to the sample (1 ml gel/5 mg protein). After incubation overnight at room temperature for 10-24 hours the resin is collected by centrifugation at 1000×g for 10 min, and non-glycoproteins are removed by washing the resin 3 times with an equal volume of urea solution (8M urea/0.4M NH₄HCO₃, pH 8.3).

The proteins on the resin are denatured in urea solution at 55° C. for 30 min and subsequently washed three times in the urea solution. Following the last wash, the urea solution is removed and the resin is diluted with 3 bed volumes of water. Trypsin is added at a concentration of 1 mg of trypsin/200 mg of protein and digested at 37° C. overnight. The peptides are reduced by adding 8 mM TCEP (Pierce, Rockford, Ill.) at room temperature for 30 min, and alkylated by adding 10 mM iodoacetamide at room temperature for 30 min. The trypsin-released peptides are removed by washing the resin three times with three bed volumes of 1.5 M NaCl, 80% acetonitrile/0.1% trifluoroacetic acid (TFA), 100% methanol, and six times with 0.1 M NH₄HCO₃. N-linked glycopeptides are released from the resin by addition of N-glycosidase F (at a concentration of 1 ml of N-glycosidase F/40 mg of protein) overnight. The resin is then pelleted and the supernatant is saved. The resin is washed twice with 80% acetonitrile/0.1% TFA and the supernatants were combined. The released peptides are dried and re-suspended in 0.4% acetic acid for LC-MS/MS analysis.

The glycopeptides remaining on the beads after trypsinization are washed three times with methanol, then twice with 15% NH₄OH in water (pH>11). After adding methylisourea at 1 M in 15% NH₄OH(NH₄OH/H₂O=15/85 v/v) in 100 fold molar excess over amine groups and incubating at 55° C. for 10 minutes beads are washed twice with water, twice with DMF/pyridine/H₂O=50/10/40 (v/v/v) and re-suspended in DMF/pyridine/H₂O=50/10/40 (v/v/v). Succinic anhydride solution is added to a final concentration of 2 mg/ml and the samples are incubated at room temperature for 1 hour, followed by washing three times with DMF, three times with water, and six times with 0.1M NH₄HCO₃. The peptides are released from the beads using N-glycosidase F as describe above.

The peptides labeled with the d0 and d4 form of succinic anhydride respectively are separated by μLC, fractionated onto a MALDI sample plate and analyzed by a TOF TOF mass spectrometer (ABI). MS spectra, one from each sample spot, are collected automatically by the mass spectrometer. A quantitative software algorithm then analyzes the MS spectra to identify paired peptide peaks and to calculate abundance ratios of those paired peptides. The algorithm also generated a list of peptide masses for each spot number, which was fed back to the mass spectrometer for automated MS/MS analysis.

7.2 iTRAQ

There are 4 iTRAQ labels, and therefore up to 4 samples can be analysed in parallel. The samples are taken up in 20 μl dissolution buffer, and 1 μl of denaturant is added. 2 μl of reducing agent is then added to the samples and these are then incubated at 60° C. for 1 hr. After incubation, 1 μl of cysteine blocking agent is added to each sample, followed by incubation for 10 mins at room temperature.

A vial of trypsin is reconstituted with 25 μl of water. To each tube, 10 ul of the trypsin solution is added. The tubes are then incubated at 37° C. overnight.

Each vial of iTRAQ reagent is reconstituted in 70 μl ethanol (after allowing the vials to come to room temperature). The contents of one tube are transferred to one sample vial. This is repeated for each sample. The tubes are then incubated at room temperature for 1 hr. The contents of all tubes are combined into a single tube.

Prior to LC-MS analysis, the sample should be cleaned up by cation exchange chromatography. The sample is acidified (to between pH 2.5 and 3.3) by addition of at least 10 fold volume of cation exchange buffer-load. The cation exchange cartridge is conditioned by injection of 1 ml of cation exchange buffer—clean, followed by 2 ml of cation exchange buffer-load.

The sample is loaded slowly onto the column, and the flow-through is collected. A further 1 ml of cation exchange buffer-load is injected to wash all excess reagents from the cartridge.

The peptides are eluted by injection of 500 μl of cation exchange buffer-elute. The eluate is collected in a fresh sample tube.

The cartridge is regenerated by washing with 1 ml of cation exchange buffer-clean. The peptides are now ready for LC-MS analysis.

### 7.3 Ordered Peptide Array

Stable isotope labeled peptides are characterized by analysis on MALDI-MS and LC-QTOF. These reference peptide stocks are then quantified by amino acid analysis.

The sample to be analysed is proteolytically digested, and optionally fractionated (e.g. by glycocapture). A precisely known amount of the reference peptide pool is then added to the sample, which is then submitted for LC fractionation, the fractions being directly spotted onto a MALDI target plate. A detailed method has been described elsewhere by Zhang et al., Nature Biotechnology, June 2003, Vol. 21, Num. 6, p. 660-666. Quantitation is carried out by comparing intensities of the reference peptide peak and the matched sample peak. An algorithm is used in order to account for the fact that a single peptide may be spread over several neighbouring spots.

### 7.4 Results

Using the above-described protocol, the inventors were able to identify the presence of glycosylation on Asparagine 266 (the numbering refers to the sequence of FIG. 2).

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A method for screening for or diagnosis or prognosis of a neurological disorder associated with de-regulated glutamate signalling in a subject, for determining the stage or severity of such a neurological disorder in a subject, for identifying a subject at risk of developing such a neurological disorder, or for monitoring the effect of therapy administered to a subject having such a neurological disorder, said method comprising:
(a) analyzing a test sample of body fluid or tissue from the subject said sample comprising at least one Protein Isoform selected from the following Protein Isoform Families: PIF-1, PIF-2, and PIF-3 in a detectable amount; and
(b) comparing the abundance of said Protein Isoform(s) in the test sample or the abundance of said Protein Isoform(s) relative to another Protein Isoform with the abundance or relative abundance of said Protein Isoform(s) in a test sample from one or more persons free from neurological disorder, or with a previously determined reference range for that Protein Isoform in subjects free from neurological disorder, wherein a diagnosis of or a positive result in screening for or a prognosis of a more advanced condition of said neurological disorder is indicated by (i) decreased abundance or relative abundance of PIF-1 and/or (ii) increased abundance or relative abundance of PIF-2 and/or (iii) decreased abundance or relative abundance of PIF-3.

2. A method according to claim 1 wherein the sample is a sample of CSF or brain tissue.

3. A method according to claim 1 wherein the sample is a sample of blood or urine.

4. A method according to claim 1 wherein the neurological disorder is selected from Alzheimer's disease, stroke, multiple sclerosis and schizophrenia.

5. A method according to claim 1 wherein a diagnosis of or a positive result in screening for or a prognosis of a more advanced condition of said neurological disorder is indicated by (i) decreased abundance of PIF-1 and/or (ii) increased abundance of PIF-2.

6. A method according to claim 1 wherein the analysis of step (a) is performed by two dimensional electrophoresis to generate a two-dimensional array of features.

7. The method according to claim 6, wherein step (a) comprises isoelectric focussing followed by sodium dodecyl sulfate polyacrylamide gel electrophoresis (SDS-PAGE).

8. The method of claim 1, wherein step (b) comprises quantitatively detecting one or more Protein Isoform(s) of the following Protein Isoform Families: PIF-1, PIF-2, and PIF-3.

9. The method according to claim 8, wherein the step of quantitatively detecting comprises testing at least one aliquot of the sample, said step of testing comprising:
(a) contacting the aliquot with an antibody or other affinity reagent such as an Affibody that is immunospecific for a preselected Protein Isoform; and
(b) quantitatively measuring any binding that has occurred between the antibody and at, least one species in the aliquot.

10. The method according to claim 9, wherein the affinity reagent is a monoclonal antibody.

11. The method according to claim 9, wherein the step of quantitatively detecting comprises testing a plurality of aliquots with a plurality of antibodies or other affinity reagents such as Affibodies for quantitative detection of a plurality of preselected Protein Isoforms.

12. The method according to claim 11, wherein the affinity reagents are monoclonal antibodies.

13. The method of claim 1, wherein the subject is treated with a drug acting on the glutamate pathway and said method is used to optimize the treatment.

14. The method of claim 1, wherein said method is used to stratify patients for evaluation of, testing of, or treatment with, a drug acting on the glutamate pathway.

15. A protein having the amino acid sequence corresponding to FIG. 2 having at its N terminus a residue within the underlined region (i.e. NL . . . IK) and finishing at the C-terminus of FIG. 2.

16. A protein having the amino acid sequence corresponding to FIG. 2 starting at the N terminus and having at its C terminus a residue within the underlined region (i.e. NL . . . IK).

17. An antibody or other affinity reagent such as an Affibody which binds immunospecifically to the protein of claim 15.

18. An affinity reagent according to claim 17 which is a monoclonal antibody.

19. An antibody or other affinity reagent such as an Affibody which binds immunospecifically to the protein of claim 16.

20. An affinity reagent according to claim 19 which is a monoclonal antibody.

21. A pharmaceutical preparation comprising a protein from Protein Isoform Family: PIF-3.

22. A pharmaceutical preparation comprising a protein from Protein Isoform Family: PIF-2.

23. A kit for screening for or diagnosis or prognosis of a neurological disorder associated with over activation of glutamate signalling in a subject which kit comprises an antibody or other affinity reagent such as an Affibody according to claim 17.

24. A kit for screening for or diagnosis or prognosis of a neurological disorder associated with over activation of glutamate signalling in a subject which kit comprises an antibody or other affinity reagent such as an Affibody which binds immunoselectively to a protein of Protein Isoform Family PIF-1.

25. A kit for screening for or diagnosis or prognosis of a neurological disorder associated with over activation of glutamate signalling in a subject which kit comprises an antibody or other affinity reagent such as an Affibody which binds immunoselectively to a protein of Protein Isoform Family PIF-2.

26. A kit for screening for or diagnosis or prognosis of a neurological disorder associated with over activation of glutamate signalling in a subject which kit comprises an antibody or other affinity reagent such as an Affibody which binds immunoselectively to a protein of Protein Isoform Family PIF-3.

27. A kit according to claim 23 wherein the affinity reagent is a monoclonal antibody.

28. A pharmaceutical composition comprising a therapeutically effective amount of an antibody or other affinity reagent of claim 19 and a pharmaceutically acceptable carrier.

29. A pharmaceutical composition comprising:
a therapeutically effective amount of a fragment or derivative of an antibody or other affinity reagent such as an Affibody of claim 19, said fragment or derivative containing the binding domain of the antibody or other affinity reagent; and
a pharmaceutically acceptable carrier.

30. A method of treating or preventing neurological disorder associated with over activation of glutamate signalling comprising administering to a subject in need of such treatment or prevention a therapeutically effective amount of a nucleic acid encoding a protein of Protein Isoform Family: PIF-3.

31. A method of treating or preventing neurological disorder associated with over activation of glutamate signalling comprising administering to a subject in need of such treatment or prevention a therapeutically effective amount of a nucleic acid encoding a protein of Protein Isoform Family: PIF-2.

32. A method of treating or preventing neurological disorder disorder associated with over activation of glutamate signalling comprising administering to a subject in need of such treatment or prevention a therapeutically effective amount of a nucleic acid that inhibits the function of a protein of Protein Isoform Family: PIF-3.

33. A method of treating or preventing neurological disorder disorder associated with over activation of glutamate signalling comprising administering to a subject in need of such treatment or prevention a therapeutically effective amount of a nucleic acid that inhibits the function of a protein of Protein Isoform Family: PIF-2.

34. The method of claim 30, wherein the nucleic acid is a Protein Isoform antisense nucleic acid or ribozyme.

35. A method of screening for agents that interact with a protein of Protein Isoform Families PIF-1, PIF-2 and/or PIF-3, a biologically active portion of said protein, or a Protein Isoform-related polypeptide thereof, said method comprising:
(a) contacting said Protein Isoform Family protein, biologically active portion, or polypeptide with a candidate agent; and
(b) determining whether or not, the candidate agent interacts with said Protein Isoform Family protein, biologically active portion, or polypeptide

36. The method of claim 35, wherein the Protein Isoform Family protein, the portion or the Protein Isoform-related polypeptide is expressed by cells.

37. The method of claim 36, wherein the cells express a recombinant Protein Isoform Family protein, a biologically active portion of said Protein Isoform Family member, or a Protein Isoform-related polypeptide thereof.

38. A method of screening for agents that modulate the expression or activity of a protein of Protein Isoform Families PIF-1, PIF-2 and/or PIF-3 or such a Protein Isoform-related polypeptide comprising:
(a) contacting a first population of cells expressing a Protein Isoform Family protein or a Protein Isoform-related polypeptide with a candidate agent;
(b) contacting a second population of cells expressing said Protein Isoform Family protein or said Protein Isoform-related polypeptide with a control agent; and
(c) comparing the level of said Protein Isoform Family protein or said Protein Isoform-related polypeptide or mRNA encoding said Protein Isoform Family protein or said Protein Isoform-related polypeptide in the first and second populations of cells, or comparing the level of induction of a cellular second messenger in the first and second populations of cells.

39. The method of claim 38, wherein the level of said Protein Isoform Family protein or said Protein Isoform-related polypeptide, mRNA encoding said Protein Isoform Family protein or said Protein Isoform-related polypeptide, or said cellular second messenger is greater in the first population of cells than in the second population of cells.

40. The method of claim 38 wherein the level of said Protein Isoform Family protein or said Protein Isoform-related polypeptide, mRNA encoding said Protein Isoform Family protein or said Protein Isoform-related polypeptide, or said cellular second messenger is less in the first population of cells than in the second population of cells.

41. A method of screening for or identifying agents that modulate the expression or activity of a protein of Protein Isoform Families PIF-1, PIF-2 and/or PIF-3 or such a Protein Isoform-related polypeptide comprising:
(a) administering a candidate agent to a first mammal or group of mammals;
(b) administering a control agent to a second mammal or group of mammals; and
(c) comparing the level of expression of the Protein Isoform Family protein or the Protein Isoform-related polypeptide or of mRNA encoding the Protein Isoform Family protein or the Protein Isoform-related polypeptide in the first and second groups, or comparing the level of induction of a cellular second messenger in the first and second groups.

42. The method of claim 41, wherein the mammals are animal models for neurological disorder.

43. The method of claim 41, wherein the level of expression of said Protein Isoform Family protein or said Protein Isoform-related polypeptide, mRNA encoding said Protein Isoform Family protein or said Protein Isoform-related polypeptide, or of said cellular second messenger is greater in the first group than in the second group.

44. The method of claim 41, wherein the level of expression of said Protein Isoform Family protein or said Protein Isoform-related polypeptide, mRNA encoding said
Protein Isoform Family protein or said Protein Isoform-related polypeptide, or of said cellular second messenger is less than in the first group than in the second group.

45. The method of claim 41, wherein the levels of said Protein Isoform Family protein or said Protein Isoform-related polypeptide, mRNA encoding said Protein Isoform Family protein or said Protein Isoform-related polypeptide, or of said cellular second messenger in the first and second groups are further compared to the level of said Protein Isoform Family protein or said Protein Isoform-related polypeptide or said mRNA encoding said Protein Isoform Family protein or said Protein Isoform-related polypeptide in normal control mammals.

46. The method of claim 36, wherein administration of said candidate agent modulates the level of said Protein Isoform Family protein or said Protein Isoform-related polypeptide, or said mRNA encoding said Protein Isoform Family protein or said Protein Isoform-related polypeptide, or said cellular second messenger in the first group towards the levels of said Protein Isoform Family protein or said Protein Isoform-related polypeptide or said mRNA or said cellular second messenger in the second group.

47. The method of claim 41, wherein said mammals are human subjects having neurological disorder.

48. A method of screening for or identifying agents that interact with a protein of Protein Isoform Families PIF-1, PIF-2 and/or PIF-3 or such a Protein Isoform-related polypeptide, comprising
(a) contacting a candidate agent with the Protein Isoform Family protein or the Protein Isoform-related polypeptide, and
(b) quantitatively detecting binding, if any, between the agent and the Protein Isoform Family protein or the Protein Isoform-related polypeptide.

49. A method of screening for or identifying agents that modulate the activity of a protein of Protein Isoform Families PIF-1, PIF-2 and/or PIF-3 or such a Protein Isoform-related polypeptide, comprising
(a) in a first aliquot, contacting a candidate agent with the Protein Isoform Family protein or the Protein Isoform-related polypeptide, and
(b) comparing the activity of the Protein Isoform Family protein or the Protein Isoform-related polypeptide in the first aliquot after addition of the candidate agent with the activity of the Protein Isoform Family protein or the Protein Isoform-related polypeptide in a control aliquot, or with a previously determined reference range.

50. The method according to claim 48, wherein the Protein Isoform Family protein or the Protein Isoform-related polypeptide is recombinant protein.

51. The method according to claim 48, wherein the Protein Isoform Family protein or the Protein Isoform-related polypeptide is immobilized on a solid phase.

52. The method according to claim 35 wherein said agents are for the treatment or prophylaxis of a neurological disorder.

53. A method for screening, diagnosis or prognosis of neurological disorder in a subject or for monitoring the effect of an anti-neurological disorder drug or therapy administered to a subject, comprising:
(a) contacting at least one oligonucleotide probe comprising 10 or more consecutive nucleotides complementary to a nucleotide sequence encoding a protein from one of the following Protein Isoform Families: PIF-1, PIF-2, and PIF-3 with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

54. The method of claim 53, wherein step (a) comprises contacting a plurality of oligonucleotide probes comprising 10 or more consecutive nucleotides complementary to a nucleotide sequence encoding a protein from one of the following Protein Isoform Families: PIF-1, PIF-2, and PIF-3 with an RNA obtained from a biological sample from the subject or with cDNA copied from the RNA wherein said contacting occurs under conditions that permit hybridization of the probe to the nucleotide sequence if present.

55. The method of claim 53, wherein step (a) includes the step of hybridizing the nucleotide sequence to a DNA array, wherein one or more members of the array are the probes complementary to a plurality of nucleotide sequences encoding distinct Protein Isoforms.

56. A method, kit, affinity reagent or preparation according to claim 1 wherein the Protein Isoform Family PIF-1 is defined by proteins of sequence corresponding to that shown in FIG. 2, and proteins having substantial identity thereto, including all isoforms thereof.

57. A method, kit, affinity reagent or preparation according to claim 1 wherein the Protein Isoform Family PIF-2 is defined by proteins of sequence corresponding to the C-terminus of the protein shown in FIG. 2 having as its N terminus a residue between residues 116 and 132, and proteins having substantial identity thereto, including all isoforms thereof.

58. A method, kit, affinity reagent or preparation according to claim 1 wherein the Protein Isoform Family PIF-3 is defined by proteins of sequence corresponding to the N-terminus of the protein shown in FIG. 2 having as its C terminus a residue between residues 116 and 132, and proteins having substantial identity thereto, including all isoforms thereof.

59. A method, kit, affinity reagent or preparation according to claim 1 wherein the Protein Isoform Family PIF-2 is defined by proteins comprising one or more sequences selected from SEQ ID Nos 16, 30, 34, 19, 8, 12, 17, 41, 45, 43, 38, 25, 29, 24, 15, 28 and 7.

60. A method, kit, affinity reagent or preparation according to claim 54 wherein the Protein Isoform Family PIF-2 is further defined by proteins not comprising a sequence selected from SEQ ID Nos 18, 56, 35, 1 and 9.

61. A method, kit, affinity reagent or preparation according to claim 1 wherein the Protein Isoform Family PIF-3 is defined by proteins comprising one or more sequence selected from SEQ ID Nos 18, 56, 35, 1, 9 and 16.

62. A method, kit, affinity reagent or preparation according to claim 56 wherein the Protein Isoform Family PIF-2 is further defined by proteins not comprising a sequence selected from SEQ ID Nos 30, 34, 19, 8, 12, 17, 41, 45, 43, 38, 25, 29, 24, 15, 28 and 7.

63. A method, kit, affinity reagent or preparation according to claim 1 wherein the Protein Isoform Family PIF-1 is defined by protein isoforms having a pI and MW as shown in Table 1 rows 1-19.

64. A method, kit, affinity reagent or preparation according to claim 1 wherein the Protein Isoform Family PIF-2 is defined by protein isoforms having a pI and MW as shown in Table 1 rows 20-33.

65. A method, kit, affinity reagent or preparation according to claim 1 wherein the Protein Isoform Family PIF-3 is defined by protein isoforms having a pI and MW as shown in Table 1 rows 34-36.

66. A method according to claim 1 wherein the method of determining the abundance of a Protein Isoform, for example a method of quantitatively detecting a Protein Isoform, involves use of an imaging technology.

67. A method according to claim 66 wherein the imaging technology involves use of labelled Affibodies.

68. A method according to claim 66 wherein the imaging technology involves use of labelled antibodies.
